(19) United States
(12) Patent Application Publication
(10) Pub. No.: US 2009/0041945 A1
(43) Pub. Date: Feb. 12, 2009

(54) ACTIVE RAY CURABLE COMPOSITION, POLYMERIZATION METHOD, ACTIVE RAY CURABLE INK, IMAGE FORMING METHOD, AND INK-JET RECORDING APPARATUS

(75) Inventors: Kimihiko Ookubo, Tokyo (JP); Norio Miura, Kanagawa (JP); Takeshi Kurata, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA MEDICAL & GRAPHIC, INC., Tokyo (JP)

(21) Appl. No.: 11/908,315

(22) PCT Filed: Mar. 15, 2006
(86) PCT No.: PCT/JP2006/305110

§ 371 Date: Sep. 11, 2007

(30) Foreign Application Priority Data
Mar. 18, 2005 (JP) 2005-079198

(51) Int. Cl. C09D 11/10; C07D 331/02; C07D 327/02; C07D 303/04; C07D 303/06; C08F 2/46; C08J 7/04

(52) U.S. Cl. 427/511; 549/90; 549/1; 549/547; 549/543; 522/31; 522/168; 522/170; 528/380; 528/417; 524/609; 524/612

(57) ABSTRACT

An active ray curable composition comprising a compound represented by Formula (X):

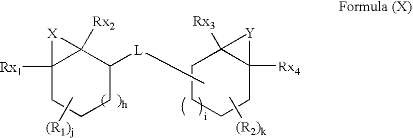

### FIELD OF THE INVENTION

The present invention relates to an active ray curable composition, which is cured for high sensitivity without depending on special environmental conditions during polymerization, as well as exhibiting excellent storage stability, and further the obtained cured layer exhibits excellent adhesion and flexibility, and yet further relates to the active ray curable composition which can provide a cured layer exhibiting sufficient hardness before and after long term storage, and also relates to the active ray curable ink (hereinafter, referred to as an ink-jet ink or an ink) using the same, and an image forming method and ink-jet recording apparatus.

### BACKGROUND OF THE INVENTION

Heretofore, an active ray curable composition, which is curedvia active rays such as ultraviolet rays and electron rays; or by heat, has been utilized in practice in various applications such as paints, adhesives and printing inks for application on such as plastics, paper, wood and inorganic materials, as well as printed circuit boards and materials serving as electrical insulation. In recent years, among such as printing inks, paints and adhesives among them, further improvement of weather resistance and adhesive capabilities has been sought. Further, as an ink-jet ink system utilizing a polymerizable composition, an ultraviolet ray curable ink-jet ink, which is cured via ultraviolet rays, is known. An ink-jet method utilizing this ultraviolet ray curable ink has attracted attention in recent years due to its relatively low odor, rapid drying and capability of recording on a non-absorptive recording medium, and ultraviolet ray curable ink-jet inks have been created (for example, please refer to Patent Documents 1 and 2). In this field, it is essential that formed film exhibits higher strength and flexibility in addition to low viscosity of the ink. It has been proposed to provide ink-jet ink of higher plasticity by addition of a plastisizer (please refer, for example, to Patent Document 3). However, the above is for melt-type ink and there is no description with respect to ultraviolet ray curable ink-jet ink utilizing solvent-type ink.

Further, when utilizing these inks, there is a problem that curing sensitivity tends to vary depending on the type of recording material and working environment.

Since an ink utilizing a radical polymerizable compound suffers from an oxygen inhibition effect, curing inhibition tends to be caused in the case of a low ink droplet volume. Further, an ink utilizing a cationic polymerizable compound (for example, please refer to Patent Documents 4-7) does not suffer from the above oxygen inhibition effect, however, there is another problem of it being easily affected by water content (moisture) at the molecular level.

[Patent Document 1] Unexamined Japanese Patent Application Publication No. (hereinafter, referred to as JP-A) 6-200204 (Claims and Examples)

[Patent Document 2] Japanese Translation of PCT International Application Publication No. 2000-504778 (Claims and Examples)

[Patent Document 3] JP-A 8-3493 (Claims and Examples)

[Patent Document 4] JP-A 2001-220526 (Claims and Examples)

[Patent Document 5] JP-A 2002-188025 (Claims and Examples)

[Patent Document 6] JP-A 2002-317139 (Claims and Examples)

[Patent Document 7] JP-A 2003-55449 (Claims and Examples)

### SUMMARY OF THE INVENTION

### Problems to be Solved

This invention is presented in view of the above problems, and an object of this invention is to provide an active ray curable composition which exhibits excellent storage stability, low viscosity, high sensitivity and formation of a cured film exhibiting excellent hardness and superior weather resistance under various environments, such as under high humidity ambience, and an active ray curable ink utilizing the same, an image forming method utilizing the active ray curable ink as an ink-jet ink, an ink-jet recording apparatus and a novel epoxy compound.

### Means to Solve the Problems

The above-described object of this invention can be achievable via the following constitutions.

Item 1. An active ray curable composition comprising a compound represented by following Formula (X).

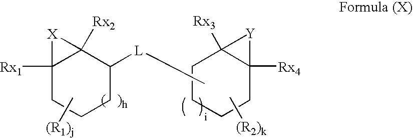

In the formula, X and Y are each independently an oxygen atom or a sulfur atom, L is a single bond or a divalent connecting group, R_{X1}, R_{X2}, R_{X3} and R_{X4} are each independently a hydrogen atom or an alkyl group, R₁ and R₂ are each independently a substituent group, h and i are each independently an integer of 0-2, j and k are each independently an integer of 0-9. “a” is 0 or 1, and when “a” is 0, L is combined to a carbon atom substituted with R_{X3}.

Item 2. The active ray curable composition described in Item 1 above, wherein in the compound represented by Formula (X), either R_{X1} or R_{X2} is an alkyl group and the other one being a hydrogen atom, and further, either R_{X3} or R_{X4} is an alkyl group and the other one is a hydrogen atom.

Item 3. The active ray curable composition described in Item 2 above, wherein in the compound represented by Formula (X), either R_{X1} or R_{X2} is an alkyl group exhibiting 1-3 carbon atoms and the other one is a hydrogen atom, and further, either R_{X3} or R_{X4} is an alkyl group exhibiting 1-3 carbon atoms and the other one is a hydrogen atom.

Item 4. The active ray curable composition described in any one of Items 1-3 above, further comprises an oxetane compound.

Item 5. The active ray curable composition described in Item 4 above, wherein the oxetane compound does not have a substituent group in a 2-position of an oxetane ring.

Item 6. The active ray curable composition described in Item 4 or 5 above, wherein the oxetane compound having no substituent group in the 2-position of the oxetane ring is a multifunctional oxetane compound having two or more oxetane rings.

Item 7. The active ray curable composition described in any one of Items 1-6 above, further comprising a compound represented by following Formula (A).

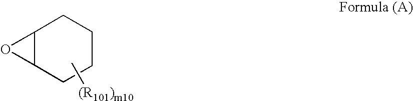

In this formula, R101 is a substituent group not having a reactive functional group of being cationic polymerizable or radical polymerizable, and m10 is 1, 2, 3, or 4.

Item 8. The active ray curable composition described in any one of Items 1-7 above, further comprises a polymerization initiator.

Item 9. The active ray curable composition described in Item 8 above, wherein the polymerization initiator is a compound which generates an acid when irradiated with active rays.

Item 10. The active ray curable composition described in Item 9 above, wherein the compound which generates an acid when irradiated with active rays is an onium salt compound.

Item 11. The active ray curable composition described in Item 10 above, wherein the compound which generates an acid when irradiated with active rays is a sulfonium salt compound.

Item 12. The active ray curable composition described in Item 11 above, wherein the sulfonium compound is one represented by following Formulas (I-1), (I-2) or (I-3).

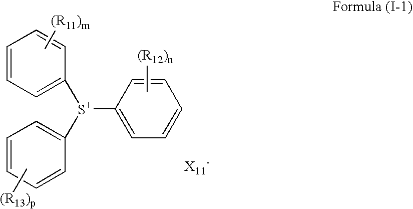

In this formula, R₁₁, R₁₂, and R₁₃ are each a substituent group, and m, n, and p are each an integer of 0-2, and X₁₁⁻ is a counter ion.

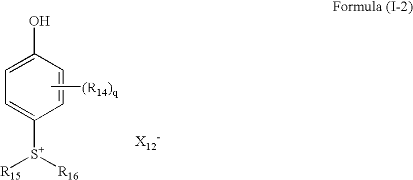

In this formula, R₁₄ is a substituent group, and q is an integer of 0-2. R₁₅ and R₁₆ are each a substituted or unsubstituted alkyl group, a substituted or unsubstituted alkenyl group, a substituted or unsubstituted alkynyl group, or a substituted or unsubstituted aryl group, and X₁₂⁻ is a counter ion.

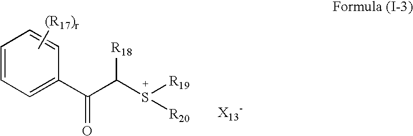

In this formula, R₁₇ is a substituent group, and r is an integer of 0-2. R₁₈ is a hydrogen atom or a substituted or unsubstituted alkyl group, and R₁₉ and R₂₀ are each a substituted or unsubstituted alkyl group, a substituted or unsubstituted alkenyl group, a substituted or unsubstituted alkynyl group, or a substituted or unsubstituted aryl group, and X₁₃⁻ is a counter ion.

Item 13. The active ray curable composition described in any one of Items 1-12 above, exhibits a viscosity of 1-500 mPa·s at 25° C.

Item 14. A polymerizing method comprising the steps of:

(a) ejecting an active ray curable composition described in any one of Items 1-13 on a recording medium, and

(b) polymerizing the ejected active ray curable composition with active rays.

Item 15. An active ray curable ink comprises an active ray curable composition described in any one of Items 1-13.

Item 16. The active ray curable ink described in Item 15 above, exhibits a viscosity of 7-40 mPa·s at 25° C.

Item 17. The active ray curable ink described in Item 15 or 16, contains a pigment.

Item 18. An image forming method comprising the steps of:

(a) ejecting imagewise an active ray curable ink described in any one of Items 15-17 onto a recording medium from an ink-jet recording head, and

(b) forming an image on the recording medium by curing the active ray curable ink,

wherein the active ray curable ink is cured by radiation of active rays during 0.001-1.0 second after deposition of the active ray curable ink onto the recording medium.

Item 19. The image forming method described in Item 18 above, wherein a minimum ink droplet volume ejected from each nozzle of the ink-jet recording head is 2-15 pl.

Item 20. The image forming method described in Item 18 or 19, wherein the active ray curable ink is ejected after the active ray curable ink and the ink-jet recording head are heated to 35-100° C.

Item 21. The image forming method described in any one of Items 18-20, wherein the active ray curable ink is ejected onto the recording medium heated to 35-60° C.

Item 22. An ink-jet recording apparatus being utilized in an image forming method described in any one of Items 18-21.

### EFFECTS OF THE INVENTION

This invention can provide an active ray curable composition, exhibiting excellent storage stability, high sensitivity and excellent hardness. The cured film of this composition exhibits sufficient hardness before and after long term storage, and further this active ray curable composition can form the cured film under varied ambience, specifically under high humidity conditions, and further can provide a polymerizable ink utilizing the same, an image forming method, and an ink-jet recording apparatus, all utilizing the same.

### BRIEF DESCRIPTION OF THE DRAWINGS

FIG. 1 shows a constitution of the primary portion of a recording apparatus utilized in this invention.

FIG. 2 shows a constitution of the primary portion of another recording apparatus utilized in this invention.

### DESCRIPTION OF THE ALPHA-NUMERIC DESIGNATIONS

1: Recording apparatus

2: Head carriage

3: Recording head

4: Irradiation means

5: Platen portion

6: Guide member

7: Bellows structure

P: Recording medium

### DESCRIPTION OF THE PREFERRED EMBODIMENT

The present invention will be further detailed in the following.

The present invention is characterized in that an active ray curable composition contains a compound represented by above Formula (X), which can provide a strong cured film unaffected by ambient humidity even with a light source of low light-intensity, and can also provide a cured film which maintains a sufficient hardness before and after long-term storage. In cases when this active ray curable composition is utilized for an active ray curable ink and an image is formed via an ink-jet recording apparatus, an excellent quality image is obtained.

The compound of this invention, represented by Formula (X), exhibits an oxirane ring or a thiirane ring, and when using a compound in which either one of two carbon atoms constituting such a ring is further substituted with an alkyl group, the inventors of this invention found that the cured film maintained sufficient hardness before and after long-term storage, in addition to further enhancement of hardness. Additionally, it is possible to obtain an active ray curable compound exhibiting high activity and excellent storage stability of hardness of the cured film.

In the active ray curable composition of this invention, in the case of further containing an oxetane compound, further enhancement of curing capability and an enhancement effect of storage stability is attained, and an active ray curable composition which exhibits high activity and excellent storage stability without adversely affecting performance after relatively long-term storage.

As an oxetane compound of this invention, using a multifunctional compound (being more than bifunctional), the active ray curable composition becomes more active, and further hardness of the cured film is enhanced.

In the active ray curable ink of this invention (hereinafter, referred to simply as the ink or the ink-jet ink), as a cationic polymerizable compound utilized with the compound represented by Formula (X) of this invention and an oxetane compound, an appropriate monofunctional epoxy compound may be employed, whereby it becomes possible to attain enhancement of more preferable reactivity, greater surface hardness of the cured film, and enhancement of other physical properties of the cured film.

In the compound represented by Formula (X), the specific bonding mode of the two cycloaliphatic structures and L is selected from the following structures [symbols in the formulas are the same groups as in Formula (X)].

Preferable bonding modes include X-Ib, X-Ic, X-IIb, X-IIc, X-IIIb, X-IIIc, and X-IIId.

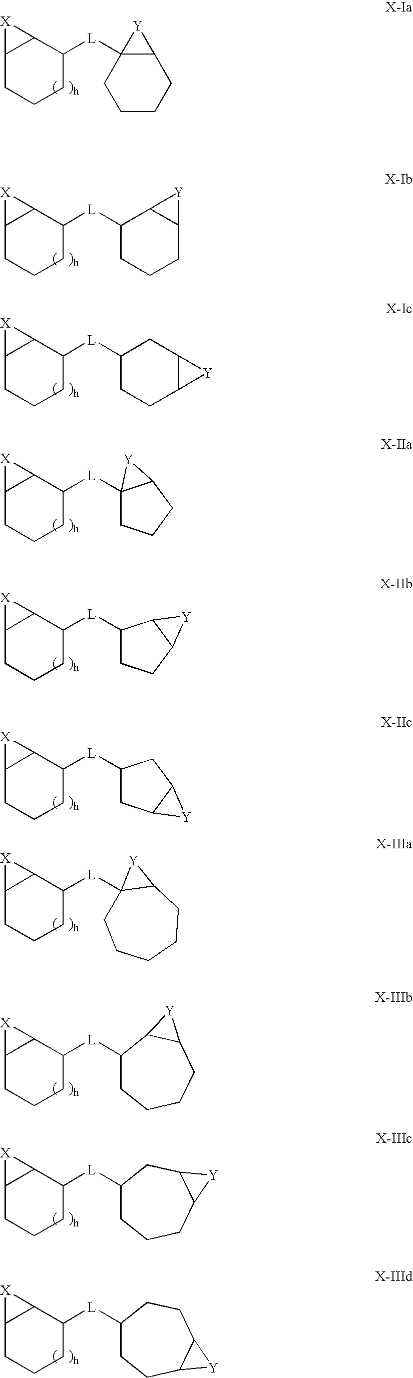

In Formula (X), X and Y are each independently an oxygen atom or a sulfur atom, and preferably either X or Y is an oxygen atom, but most preferably both X and Y are oxygen atoms. L is a simple connecting group or a divalent connecting group, and if a single connecting group, the two ring structures are directly bonded via the connecting group.

When L is a divalent connecting group, cited as a divalent connecting group may be a —O— group, a —S— group, a —CO— group, a —SO— group, a —SO₂— group, a mono-substituted amino group, an alkylene group, an arylene group, and groups combined of plural groups thereof.

As a preferable divalent connecting group represented by L, cited are an oxygen atom, a sulfur atom, a divalent connecting group having a carbon number of 1-15 in which the connecting group may contain an oxygen atom or a sulfur atom in the main chain. Examples of a divalent connecting group having a carbon number of 1-15 and having an oxygen atom or a sulfur atom in the main chain include the following groups and groups consisting of a plural of combined groups of these groups, as well as a —O— group, a —S— group, a —CO— group, and a —CS— group.
Methylene group [—CH—]
Ethylidene group [>CHCH₃]
Isopropylidene group [>C(CH₃)₂]
1,2-Ethylene group [—CH₂CH₂—]
1,2-Propylene group [—CH(CH₃)CH₂—]
1,3-Propanediyl group [—CH₂CH₂CH₂—]
2,2-Dimethyl-1,3-propanediyl group [—CH₂C(CH₃)₂CH₂—]
2,2-Dimethoxy-1,3-propanediyl group [—CH₂C(OCH₃)₂CH₂—]
2,2-Dimethoxymethyl-1,3-propanediyl group [—CH₂C(CH₂OCH₃)₂CH₂—]
1-Methyl-1,3-propanediyl group [—CH(CH₃)CH₂CH₂—]
1,4-Butanediyl group [—CH₂CH₂CH₂CH₂—]
1,5-Pentanediyl group [—CH₂CH₂CH₂CH₂CH₂—]
Oxydiethylene group [—CH₂CH₂OCH₂CH₂—]
Thiodiethylene group [—CH₂CH₂SCH₉CH₂—]
3-Oxthothiodiethylene group [—CH₂CH₂SOCH₂CH₂—]
3,3-Dioxthothiodiethylene group [—CH₂CH₂SO₂CH₂CH₉—]
1,4-Dimethyl-3-oxa-1,5-pentadiyl group [—CH(CH₃)CH₂OCH(CH₃)CH₂—]
3-Oxopentanediyl group [—CH₂CH₂COCH₂CH₂—]
1,5-Dioxo-3-oxapentanediyl group [—COCH₉OCH₂CO—]
4-Oxa-1,7-heptanediyl group [—CH₂CH₂CH₂OCH₂CH₂CH₂—]
3,6-Dioxa-1,8-octanediyl group [—CH₂CH₂OCH₂CH₂OCH₂CH₂—]
1,4,7-Trimethyl-3,6-dioxa-1,8-octanediyl group [—CH(CH₃)CH₂O—CH(CH₃)CH₂OCH(CH₃) CH₂—]
5,5-Dimethyl-3,7-dioxa-1,9-nonanediyl group [—CH₂CH₂OCH₂C(CH₃)₂CH₂OCH₂CH₂—]
5,5-Dimethoxy-3,7-dioxa-1,9-nonanediyl group [—CH₂CH₂OCH₂C(OCH₃)₂CH₂OCH₂CH₂—]
5,5-Dimethoxymethyl-3,7-dioxa-1,9-nonanediyl group [—CH₂CH₂OCH₂C(CH₂OCH₃)₂CH₂OCH₂CH₂—]
4,7-Dioxo-3,8-dioxa-1,10-decanediyl group [—CH₂CH₂OCOCH₂CH₂COOCH₂CH₂—]
3,8-Dioxo-4,7-dioxa-1,10-decanediyl group [—CH₂CH₂COOCH₂CH₂OCOCH₂CH₂—]
1,3-Cyclopentanediyl group [-1,3-C₅H₈—]
1,2-Cyclohexanediyl group [-1,2-C₆H₁₀—]
1,3-Cyclohexanediyl group [-1,3-C₆H₁₀—]
1,4-Cyclohexanediyl group [-1,4-C₆H₁₀—]
2,5-Tetrahydrofurandiyl group [2,5-C₄H₆O—]
p-Phenylene group [-p-C₆H₄—]
m-Phenylene group [-m-C₆H₄—]
α,α′-o-Xylylene group [-o-CH₂—C₆H₄—CH₂—]
α,α′-m-Xylylene group [-m-CH₂—C₆H₄—CH₂—]
α,α′-p-Xylylene group [-p-CH₂—C₆H₄—CH₂—]
Furan-2,5-diyl-bismethylene group [2,5-CH₂—C₄H₂O—CH₂—]
Thiophen-2,5-diyl-bismethylene [2,5-CH₂—C₄H₂S—CH₂—]
i-Propylidenbis-p-phenylene [-p-C₆H₄—C(CH₃)₂-p-C₆H₄—]

L may be provided with a substituent group. Examples of such a substituent group include a halogen atom (such as a chlorine atom, a bromine atom, or a fluorine atom); an alkyl group having a carbon number of 1-6 (such as a methyl group, an ethyl group, a propyl group, an isopropyl group or a butyl group); an alkoxy group having a carbon number of 1-6 (such as a methoxy group, an ethoxy group, an n-propoxy group, an iso-propoxy group, an n-butoxy group or a tert-butoxy group); an acyl group (such as an acetyl group, a propionyl group or a trifluoroacetyl group); an acyloxy group (such as an acetoxy group, a propionyloxy group or a trifluoroacetoxy group); and an alkoxycarbonyl group (such as a methoxycarbonyl group, an ethoxycarbonyl group or a tert-butoxycarbonyl group). A preferable substituent group is an alkyl group, an alkoxy group or an alkoxycarbonyl group.

L is preferably a simple connecting group, a —O— group, or a divalent connecting group which may contain an oxygen atom or a sulfur atom in the main chain and having a carbon number of 1-8, but is more preferable a simple connecting group, a —O— group, a divalent connecting group the main chain of which may contain an oxygen atom, or a divalent connecting group the main chain of which is comprised of only carbon atoms and having a carbon number of 1-5.

In Formula (X), “a” is 0 or 1. In the case of “a” being 0, L bonds to the carbon atom substituted by R_{X3}.

Rₓ₁, Rₓ₂, Rₓ₃, and Rₓ₄ are each a hydrogen atom or an alkyl group, and examples of such an alkyl group include an alkyl group having a carbon number of 1-20 (such as a methyl group, an ethyl group, a propyl group, an isopropyl group, a butyl group, a pentyl group, a hexyl group, a cyclopentyl group, a cyclohexyl group, a heptyl group, an octyl group, a 2-ethylhexyl group, a decyl group, and a dodecyl group). These may further have a substituent group, examples of such a substituent group of an alkyl group having a substituent group include a halogen atom (such as a chlorine atom, a bromine atom, and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); and an alkoxycarbonyl group having a carbon number of 1-20 (such as a methoxycarbonyl group, an ethoxycarbonyl group and a tert-butoxycarbonyl group); an alkylthiocarbonyl group having a carbon number of 2-20 (such as a methylthiocarbonyl group, an ethylthiocarbonyl group, and a tert-butylthiocarbonyl group); an arloxycarbonyl group, an alkylsulfonyl group, an arylsulfonyl group, a cyano group, and a nitro group.

Rₓ₁, Rₓ₂, Rₓ₃, and Rₓ₄ are preferably each independently a hydrogen atom or an alkyl group having a carbon number of 1-6 (such as a methyl group, an ethyl group, a propyl group, and an isopropyl group, a butyl group, a pentyl group, a hexyl group), but more preferably each is independently a hydrogen atom, or a methyl group, an ethyl group, a propyl group, or an isopropyl group.

In Rₓ₁, Rₓ₂, Rₓ₃, and Rₓ₇ each of which is bonded to two carbon atoms which constitutes an oxirane ring, the case in which either of R_{X1} or R_{X2} is an alkyl group and the other is a hydrogen atom, and further, either of R_{X3} or R_{X4} is an alkyl group and the other one is a hydrogen atom, is preferable in terms of enhancement of storage stability, in addition to enhancement of curability. In this case, as an alkyl group of either R_{X1} or R_{X2} being an alkyl group and either of R_{X3} or R_{X4} being an alkyl group, a methyl group, an ethyl group, a propyl group, or an isopropyl group is preferable. Further, either of R_{X1} or R_{X2}, and either of R_{X3} or R_{X4} are most preferably methyl groups.

R₁ and R₂ are each independently a substituent group. Examples of such a group include the alkyl group mentioned above, a halogen atom (such as a chlorine atom, a bromine atom, and a fluorine atom); an alkoxy group having a carbon number of 1-20 (such as a methoxy group, an ethoxy group, an n-propoxy group, an iso-propoxy group, an n-buthoxy group, and a tert-buthoxy group); an acyl groups (such as an acetyl group, a propionyl group, and a trifluoroacetyl group); an acyloxy group having a carbon number of 1-20 (such as an acetoxy group, a propyonyloxy group, and a trifluoroacetoxy group); an alkoxycarbonyl group having a carbon number of 1-20 (such as a methoxycarbonyl group, an ethoxycarbonyl group, and a tert-butoxycarbonyl group); and an alkylthiocarbonyl group having a carbon number of 2-20 (such as a methylthiocarbonyl group, an ethylthiocarbonyl group, and a tert-butylthiocarbonyl group); an aryloxycarbonyl group, an alkylsulfonyl group, an arylsulfonyl group, a cyano group, and a nitro group. An alkyl group, an alkoxy group, and an alkoxycarbonyl group are preferable as a substituent group. Further R₁ and R₂ may each have a substituent group independently, and examples of the substituent group include the above cited groups. When there are two or more R₁ and R₂, they may be the same or different respectively, and may combine at an appropriate position to form a ring. “h” and “i” are each independently an integer of 0-2, but preferably 0 or 1. “j” and “k” are each independently an integer of 0-9, preferably an integer of 0-5, still more preferably an integer of 0-3, and most preferably 0-2.

In the following, specific examples represented by Formula (X) of this invention are shown, but this invention is not limited to those examples.

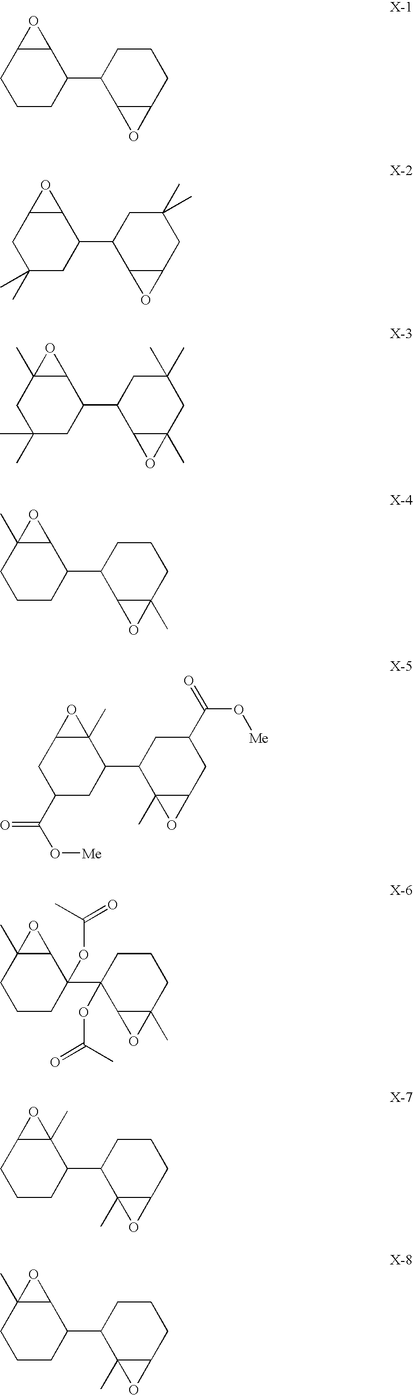

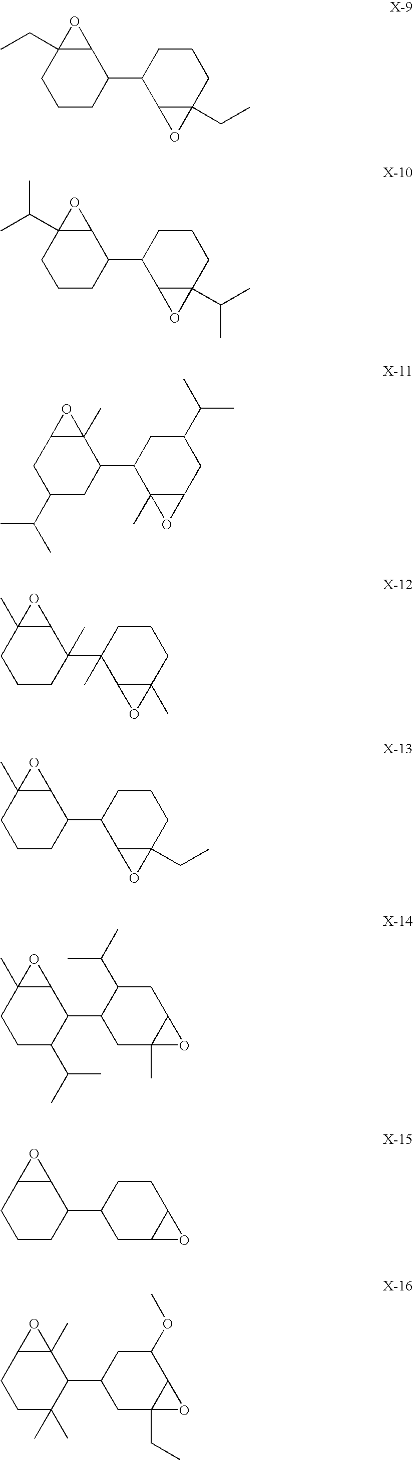

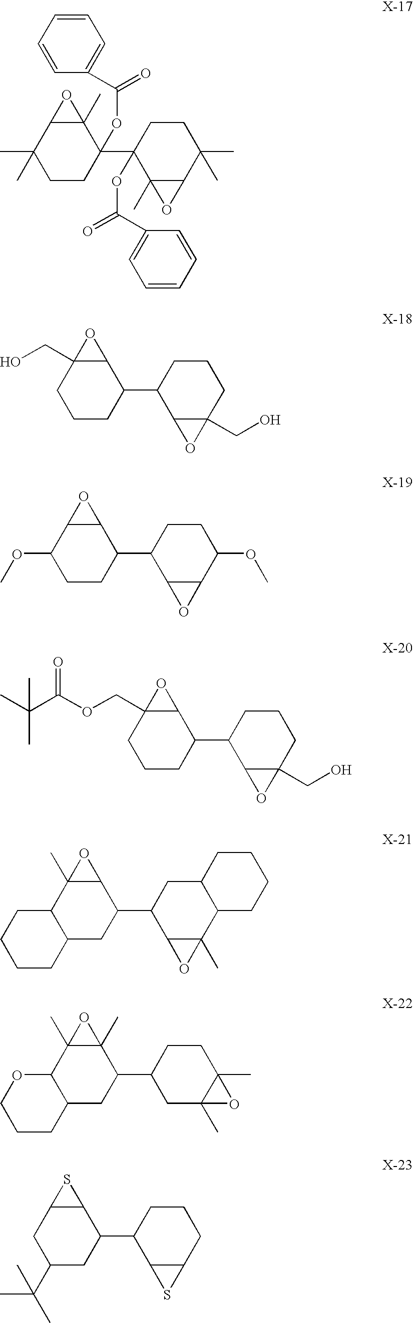

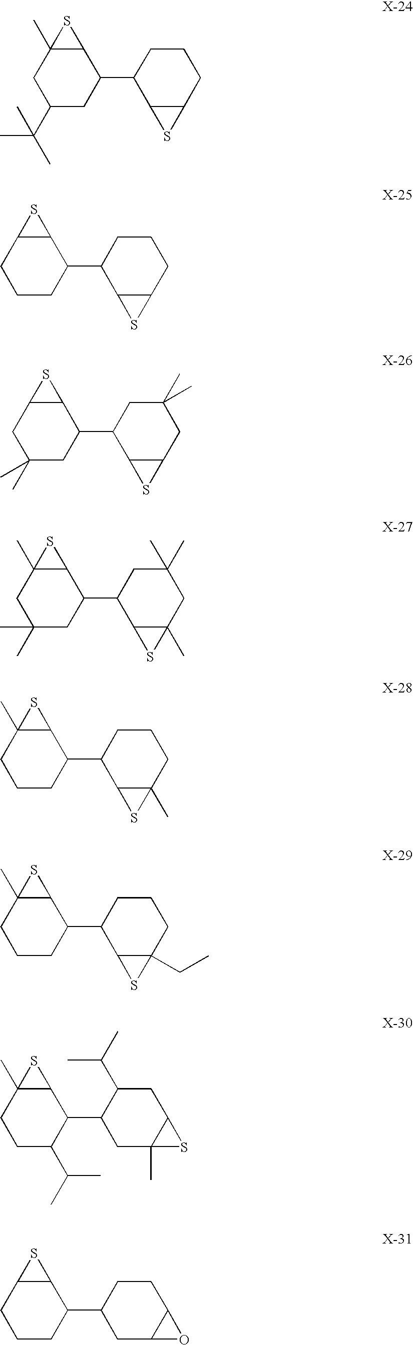

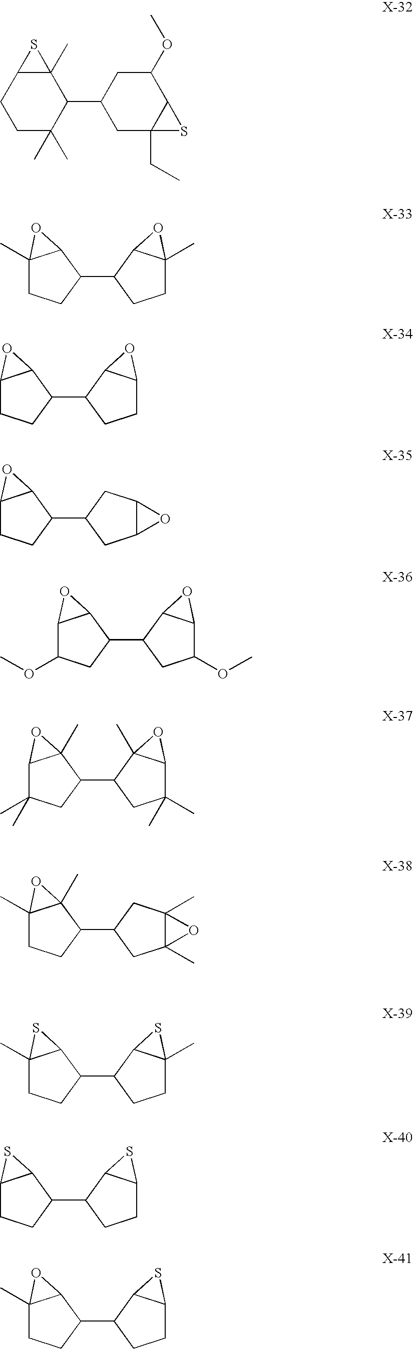

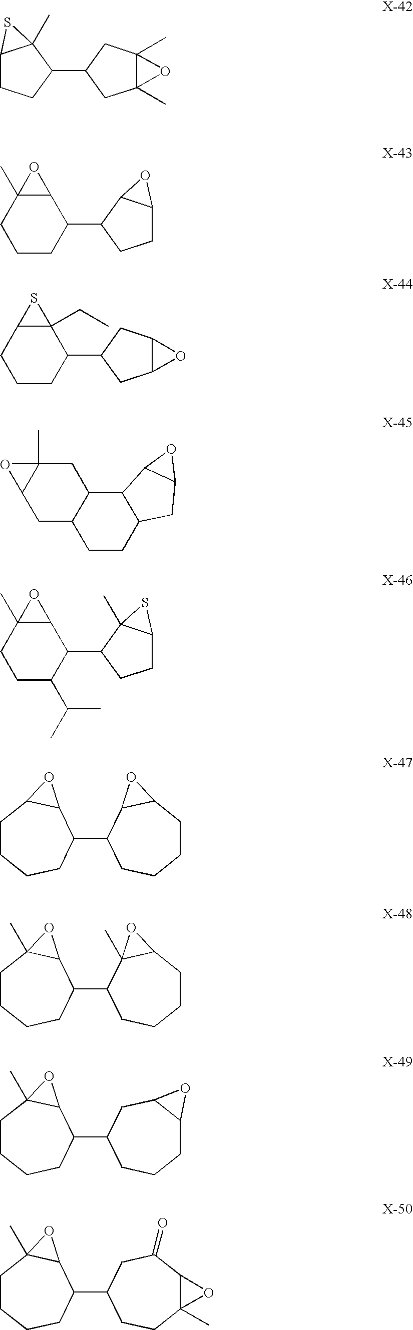

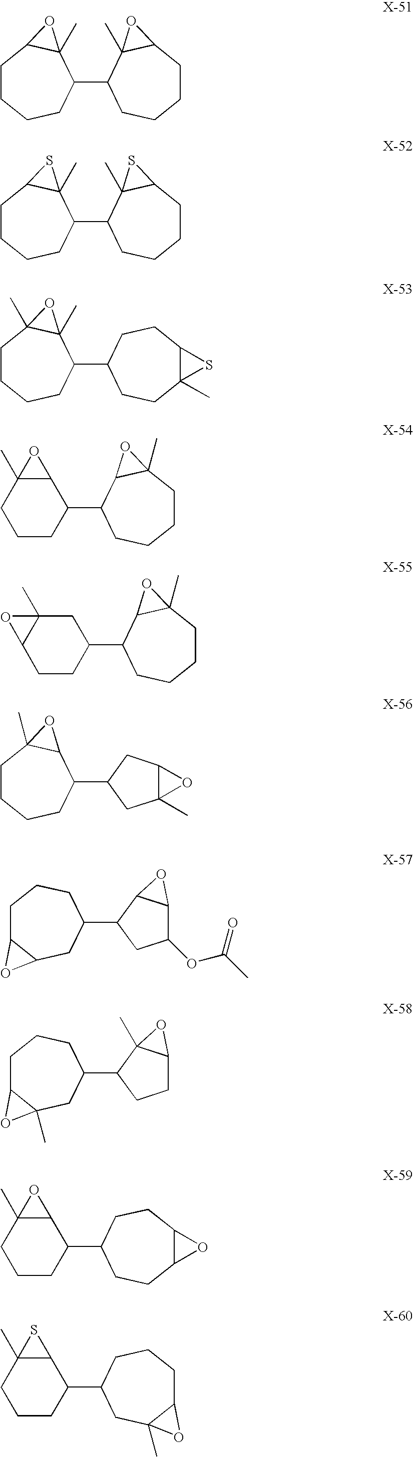

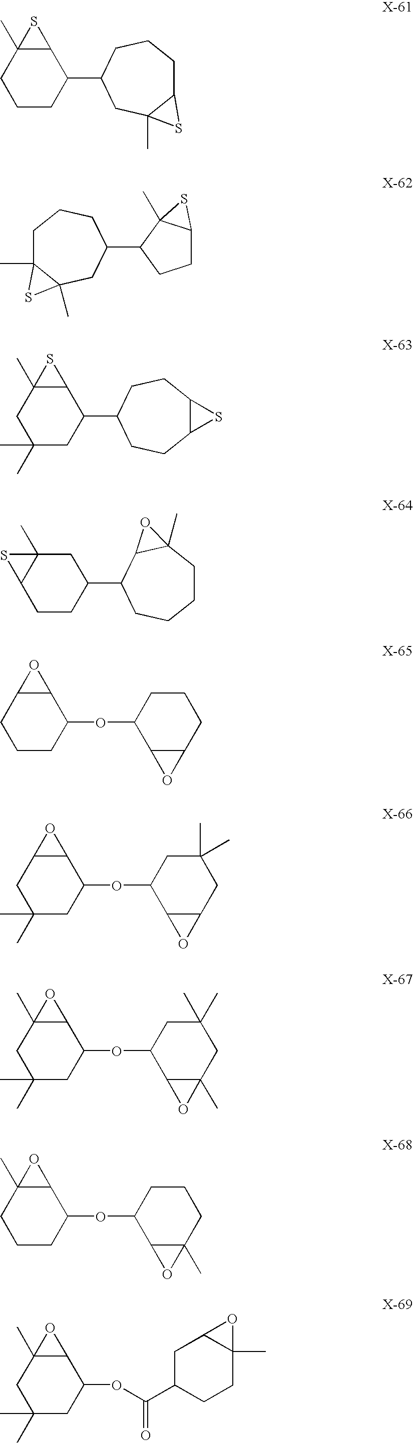

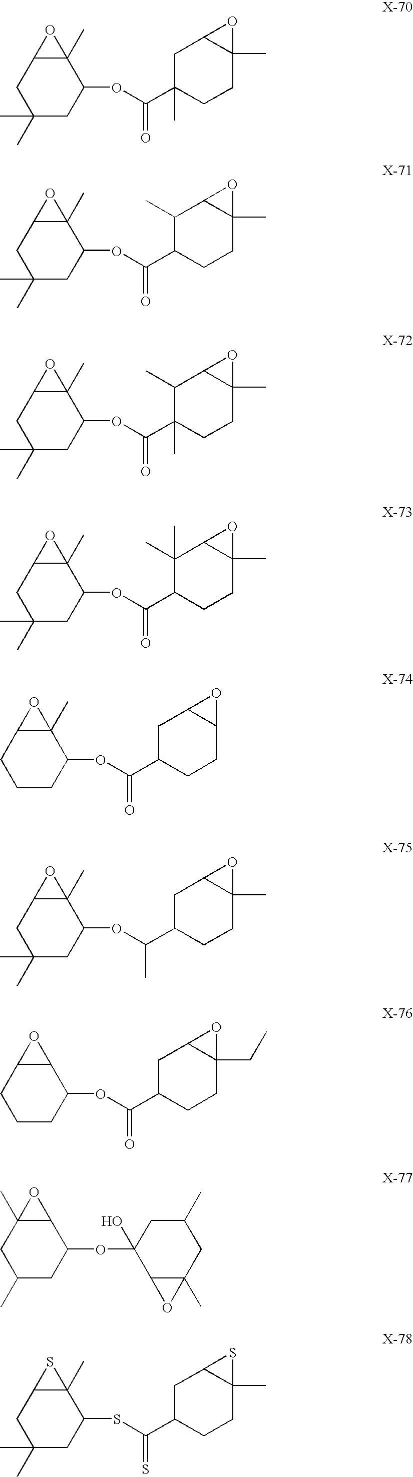

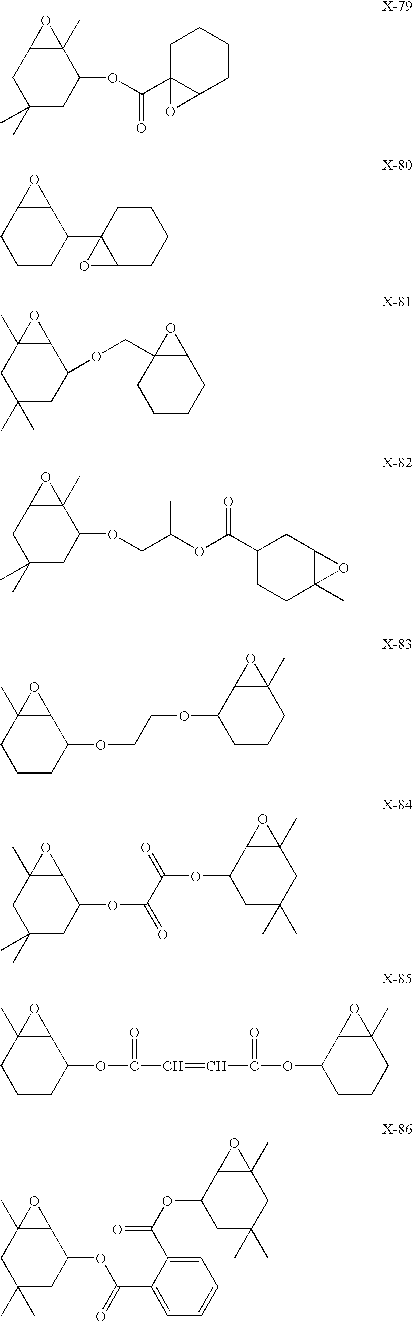

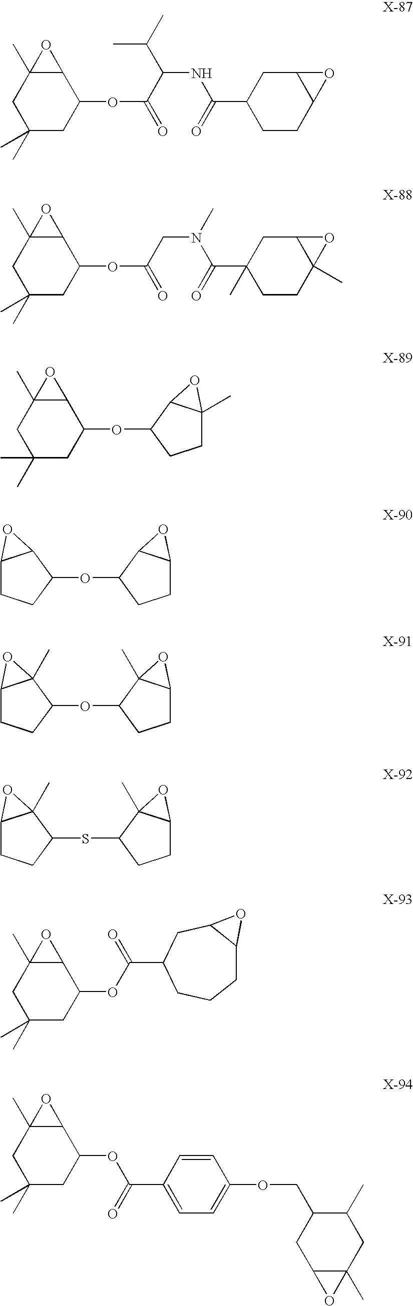

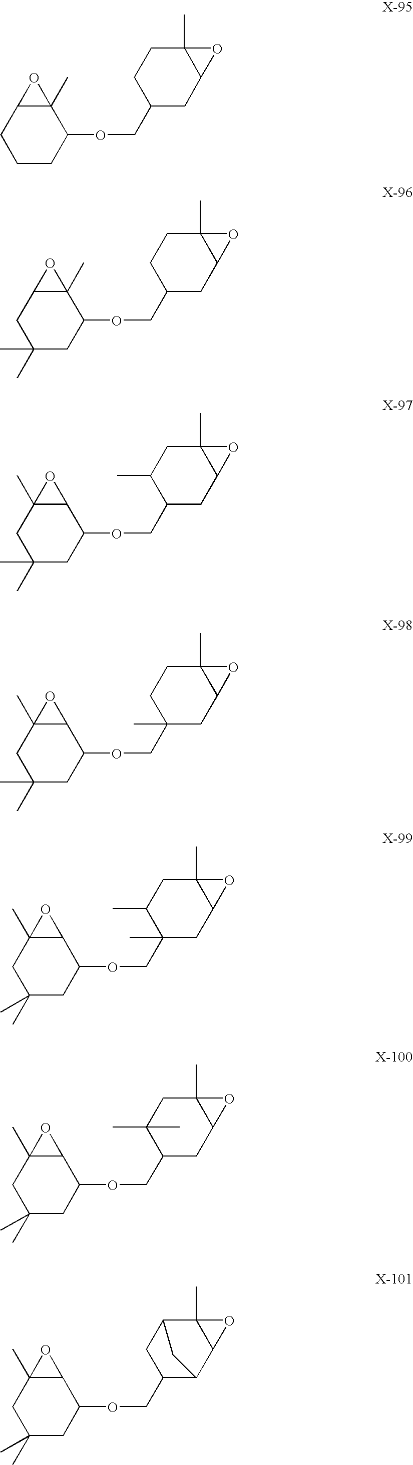

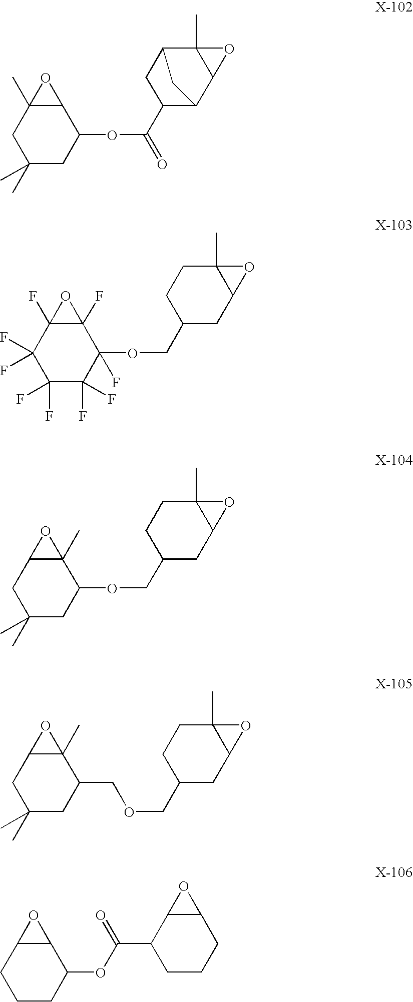

The compounds represented by Formula (X), the manufacturing method of which is not limited, can be synthesized by referring for details to, for example, J. Org. Chem., 33, 1656 (1968); J. Org. Chem., 42, 1197 (1977); J. Org. Chem., 51, 3038 (1986); J. Org. Chem., 62, 3748 (1997); and Tetrahedron Lett., 29, 4177 (1988); and also the document describing a manufacturing method of the compound represented by Formula (B) of this invention, which is to be described later.

Synthesis examples of a compound represented by Formula (X) will now be described, however, this invention is not limited thereto.

### SYNTHESIS EXAMPLE 1

### Synthesis of Exemplary Compound X-3

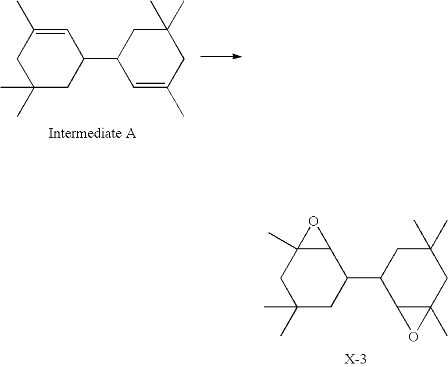

In 60 ml of methylene chloride, dissolved was 12.3 g of Intermediate A while in 100 ml of methylene chloride, dissolved was 14.6 g of m-chloro-perbenzoic acid, and then the solution was dripped into the former solution of Intermediate A over two hours. Reaction was allowed for five hours at ambient temperature, after which dissipation of the raw materials was confirmed via gas chromatography. After finishing of reaction, an appropriate amount of a sodium sulfite aqueous solution was added, after which the excessive m-chloro-perbenzoic acid was neutralized. The resulting organic layer was separated and washed several times by a sodium bicarbonate aqueous solution, and the solvent in the organic layer was evaporated under reduced pressure. The residue was purified by reduced pressure evaporation to prepare X-3. The prepared amount was 17.5 g (being a yield of 63%). The targeted product was confirmed by NMR and mass spectrum.

### SYNTHESIS EXAMPLE 2

### Synthesis of Exemplary Compound X-69

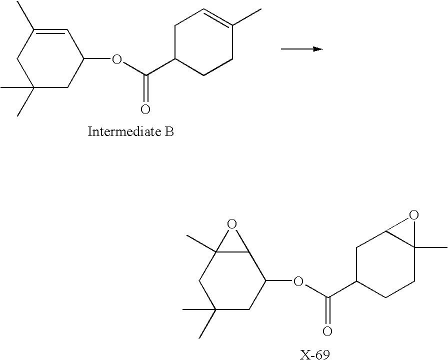

In 75 ml of methylene chloride, dissolved was 26.2 g of Intermediate B, in 100 ml of methylene chloride, dissolved was 30.0 g of m-chloro-perbenzoic acid, and then the solution was dripped into the former solution of Intermediate B over 2.5 hours. Reaction was allowed for six hours at ambient temperature, after which dissipation of the raw materials was confirmed via gas chromatography. After finishing the reaction, an appropriate amount of a sodium sulfite aqueous solution was added, after which any excessive m-chloro-perbenzoic acid was neutralized. An organic layer was separated and washed several times by a sodium bicarbonate aqueous solution, and the solvent in the organic layer was evaporated under reduced pressure. The residue was purified by reduced pressure evaporation to prepare X-69. The prepared amount was 12.1 g (being a yield of 41%). The targeted product was confirmed by NMR and mass spectrum.

### SYNTHESIS EXAMPLE 3

### Synthesis of Exemplary Compound X-96

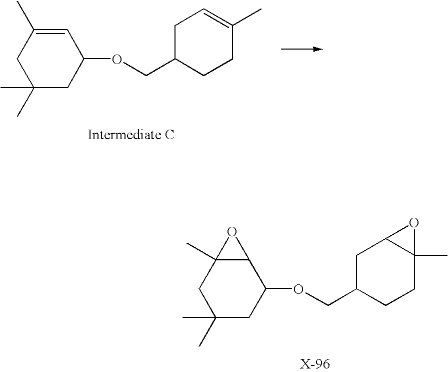

In 60 ml of methylene chloride, dissolved was 22.3 g of Intermediate C, in 80 ml of methylene chloride, dissolved was 27.5 g of m-chloro-perbenzoic acid, after which the solution was dripped into the above solution of Intermediate C over two hours. Reaction was allowed for six hours at ambient temperature, after which dissipation of the raw materials was confirmed via gas chromatography. After finishing the reaction, an appropriate amount of a sodium sulfite aqueous solution was added, after which the excessive m-chloro-perbenzoic acid was neutralized. An organic layer was separated and washed several times by a sodium bicarbonate aqueous solution, and the solvent in the organic layer was evaporated under reduced pressure. The residue was purified by reduced pressure evaporation to prepare X-96. The prepared amount was 11.8 g (being a yield of 47%). The targeted product was confirmed by NMR and mass spectrum.

The added amount of the compound represented by Formula (X) is preferably 10-70 weight % based on the total weight of the composition from the viewpoint of curing capability concerning curing conditions (being temperature and humidity) and physical properties of the film after curing, but is more preferably 20-60 weight %.

In this invention, each kind of compounds represented by Formula (X) may be employed alone, but more than two kinds of the compounds may be employed in appropriate combinations.

In an active ray curable composition of this invention, an oxetane compound, the 2-position of an oxetane ring of which is not substituted, is preferably utilized together with the compound represented by Formula (X).

In the following, an oxetane compound, the 2-position of which is not substituted, will now be described. An example of an oxetane compound, in which the 2-position is not substituted, includes compounds represented by following Formula (101).

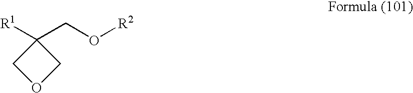

In Formula (101), R¹ is a hydrogen atom, an alkyl group having a carbon number of 1-6, such as a methyl group, an ethyl group, a propyl group and a butyl group; and a fluoroalkyl group having a carbon number of 1-6, such as an allyl group, an aryl group, a furyl group or a thienyl group. R² is an alkyl group having a carbon number of 1-6, such as a methyl group, an ethyl group, a propyl group and a butyl group; an alkenyl group having a carbon number of 2-6, such as a 1-propenyl group, a 2-propenyl group, a 2-methyl-1-propenyl group, a 2-methyl-2-propenyl group, a 1-butenyl group, a 2-butenyl group and a 3-butenyl group; a group having an aromatic ring such as a phenyl group, a benzyl group, a fluorobenzyl group, a methoxybenzyl group and a phenoxybenzyl group; an alkylcarbonyl group having a carbon number of 2-6, such as an ethylcarbonyl group, a propylcarbonyl group and a butylcarbonyl group; an alkoxycarbonyl group having a carbon number of 2-6, such as an ethoxycarbonyl group, a propoxycarbonyl group and a butoxylcarbonyl group; or an N-alkylcarbamoyl group having a carbon number of 2-6, such as an ethylcarbamoyl group, a propylcarbamoyl group, a butylcarbamoyl group and a pentylcarbamoyl group.

As an oxetane compound utilized in this invention, a compound exhibiting two oxetane rings is specifically preferable because the resulting composition exhibits superior adhesiveness and excellent working properties due to its low viscosity.

Examples of a compound exhibiting two oxetane rings include such compounds as represented by following Formula (102).

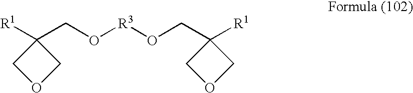

In Formula (102), R¹ is a group identical to those in previously described Formula (101). R³ is, for example, a linear or branched alkylene group such as an ethylene group, a propylene group or a butylenes group; a linear or branched poly(alkyleneoxy) group such as a poly(ethyleneoxy) group or a poly(propyleneoxy) group; a linear or branched unsaturated hydrocarbon group such as a propenylene group, a methylpropenylene group or a butenylene group; a carbonyl group or an alkylene group containing a carbonyl group; or an alkylene group containing a carboxyl group or an alkylene group containing a carbamoyl group.

Further, R³ includes a polyvalent group selected from the groups represented by following Formulas (103), (104) and (105).

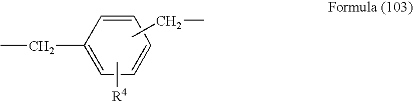

In Formula (103), R⁴ is a hydrogen atom or an alkyl group having a carbon number of 1-4 such as a methyl group, an ethyl group, a propyl group or a butyl group; an alkoxy group having a carbon number of 1-4 such as a methoxy group, an ethoxy group, a propoxy group or a butoxy group; a halogen atom such as a chlorine atom and a bromine atom; or a nitro group, a cyano group, a mercapto group, a lower alkylcarboxyl group, a carboxyl group or a carbamoyl group.

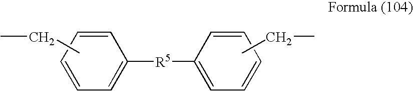

In Formula (104), R⁵ is an oxygen atom, a sulfur atom, a methylene group, NH, SO, SO₂, C(CF₃)₂, or C(CH₃)₂.

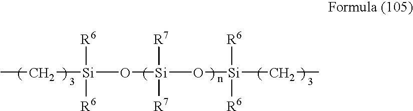

In Formula (105), R⁶ is an alkyl group having a carbon number of 1-4 such as a methyl group, an ethyl group, a propyl group or a butyl group; or an aryl group. “n” is an integer of 0-2,000. R⁷ is an alkyl group having a carbon number of 1-4 such as a methyl group, an ethyl group, a propyl group or a butyl group; or an aryl group. R⁷ also includes a group selected from the groups represented by following Formula (106).

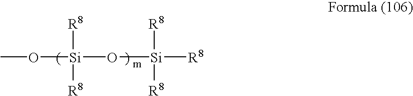

In Formula (106), R⁸ is an alkyl group having a carbon number of 1-4 such as a methyl group, an ethyl group, a propyl group or a butyl group; or an aryl group. “m” is an integer of 0-100. Specific examples of a compound having two oxetane rings include the following compounds.

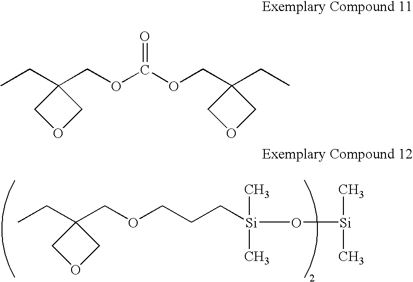

Exemplary compound 11 is one of aforesaid Formula (102) in which R² is an ethyl group and R³ is a carboxyl group. Further, exemplary compound 12 is one of aforesaid Formula (102) in which R¹ is an ethyl group, R³ is aforesaid Formula (105), in which R⁶ and R⁷ are a methyl group, and “n” is 1.

In a compound having two oxetane rings, preferable examples other than the above-described compounds include a compound represented by following Formula (107). In Formula (107), R¹ is identical to R¹ of previous Formula (101).

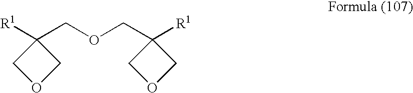

Further, examples of a compound having 3-4 oxetane rings include a compound represented by following Formula (108).

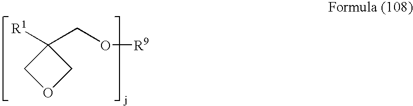

In Formula (108), R¹ is identical to R¹ of aforesaid Formula (101). R⁹ includes a branched alkylene group having a carbon number of 1-12 such as groups represented by following Groups A-C, a branched poly(alkyleneoxy) group such as groups represented by following Group D, and a branched polysiloxy group such as those represented by following Group E. “j” is 3 or 4.

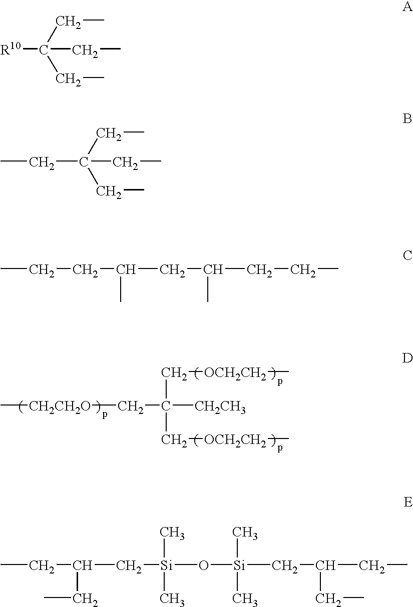

In above Group A, R¹⁰ is a lower alkyl group such as a methyl group, an ethyl group or a propyl group. Further, in above Group D, “p” is an integer of 1-10.

An example of a compound having 3-4 oxetane rings includes Exemplary Compound 13.

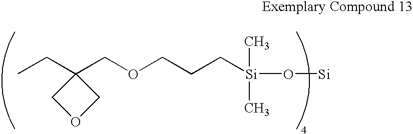

Further, examples of a compound having 1-4 oxetane rings other than those detailed above include a compound represented by following Formula (109).

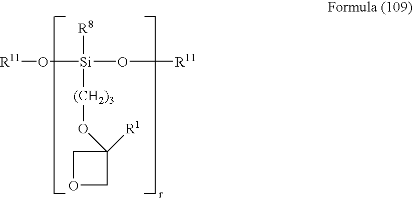

In Formula (109), R⁸ is identical to R⁸ of aforesaid Formula (106). R¹¹ is an alkyl group having a carbon number of 1-4 such as a methyl group, an ethyl group, a propyl group or a butyl group; or a trialkylsilyl group, and “r” is 1-4.

Specific examples of an oxetane compound preferably utilized in this invention include the following compounds.

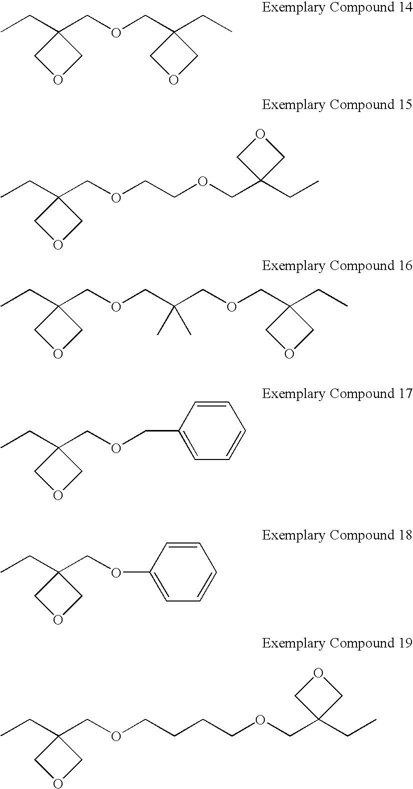

The manufacturing method of each compound having an oxetane ring described above is not specifically limited, and can be performed according to a conventional well-known method such as a synthesis method of an oxetane ring from a diol which is disclosed by D. B. Pattison, J. Am. Chem. Soc., 3455, 79 (1957). Further, in addition to these, a compound having 1-4 oxetane rings and a molecular weight of approximately 1,000-5,000 is listed. As specific example compounds thereof, the following compounds are listed.

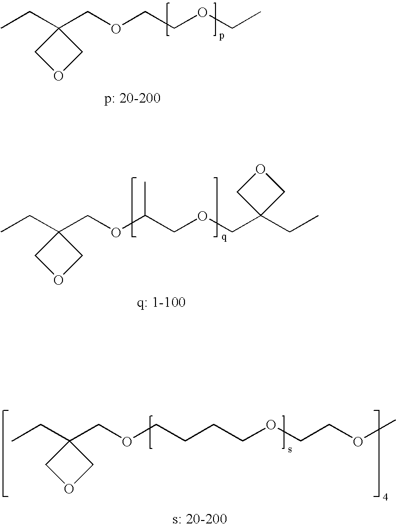

The active ray curable composition preferably contains a compound represented by above Formula (A).

In aforesaid Formula (A), R₁₀₁ is a substituent containing no functional group, which is cationically polymerizable or radical polymerizable. Examples of the substituent include a halogen atom (such as a chlorine atom, a bromine atom and a fluorine atom), an alkyl group having a carbon number of 1-20 (such as a methyl group, an ethyl group, a propyl group, an isopropyl group and a butyl group), a cycloalkyl group having a carbon number of 3-6 (such as cyclopropyl group, a cyclobutyl group, a cyclopentyl group and a cyclohexyl group); an alkoxy group having a carbon number of 1-20 (such as a methoxy group, an ethoxy group, a propoxy group, an iso-propoxy group, a n-butoxy group and a tert-butoxy group); an acyl group having a carbon number of 2-20 (such as an acetyl group, a propionyl group and a trifluoroacetyl group); an acyloxy group having a carbon number of 2-20 (such as an acetoxy group, a propionyloxy group and a trifluoroacetoxy group); an acylthio group having a carbon number of 2-20 (such as an acetylthio group, a propionylthio group and a trifluoroacetylthio group); an alkoxycarbonyl group having a carbon number of 2-20 (such as a methoxycarbonyl group, an ethoxycarbonyl group and a tert-butoxycarbonyl group); and an alkylthiocarbonyl group having a carbon number of 2-20 (such as a methylthiocarbonyl group, an ethylthiocarbonyl group and a tert-butylthiocarbonyl group).

These groups may be further provided with a substituent, such as a halogen atom (such as a chlorine atom, a bromine atom and a fluorine atom); an alkoxy group having a carbon number of 1-20 (such as a methoxy group, an ethoxy group, a n-propoxy group, an iso-propoxy group, a n-butoxy group and a tert-butoxy group); an acyl group having a carbon number of 2-20 (such as an acetyl group, a propionyl group and a trifluoroacetyl group); an acyloxy group having a carbon number of 2-20 (such as an acetoxy group, a propionyloxy group and a trifluoroacetoxy group); an alkoxycarbonyl group having a carbon number of 2-20 (such as a methoxycarbonyl group, an ethoxycarbonyl group and a tert-butoxycarbonyl group); an alkylthiocarbonyl group having a carbon number of 2-20 (such as a methylthiocarbonyl group, an ethylthiocarbonyl group and a tert-butylthiocarbonyl group); and an aryloxycarbonyl group; an alkylsulfonyl group; an arylsulfonyl group; a cyano group; and a nitro group. Preferable substituents are a halogen atom, an alkoxy group, an acyloxy group and an alkoxycarbonyl group.

A more preferable alicyclic epoxide, with respect to forming cured film having a high hardness and improving adhesion of cured film with a substrate, is a compound represented by aforesaid Formula (A-I).

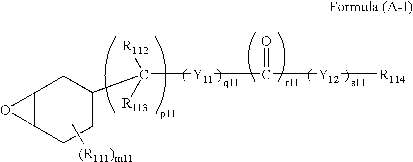

In above Formula (A-I), examples of a substituent group represented by R₁₁₁ include a halogen atom (such as a chlorine atom, a bromine atom and a fluorine atom); an alkyl group having a carbon number of 1-20 (such as a methyl group, an ethyl group, a propyl group, an isopropyl group and a butyl group); an alkoxy group having a carbon number of 1-20 (such as a methoxy group, an ethoxy group, a propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferable substituent groups are an alkyl group, an alkoxy group and an alkoxycarbonyl group.

R₁₁₂, R₁₁₃ and R₁₁₄ are a hydrogen atom or a substituted or unsubstituted alkyl group. Examples of an alkyl group include groups identical to examples of the alkyl groups of R₁₁₁ described above. Examples of a substituent group of an alkyl group having a substituent group include a halogen atom (such as a chlorine atom, a bromine atom and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); and an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferable substituent groups are an alkoxy group and an alkoxycarbonyl group.

Y₁₁ and Y₁₂ are each “O” or “S” and preferably “O”. “m11” is 0-3 and preferably 1 or 2. “p11” is 0, 1 or 2; q11, r11 and s11 are 0 or 1.

Specifically preferable alicyclic epoxide, with respect to forming cured film exhibiting high hardness and enhanced adhesion of cured film to the substrate, is a compound represented by following Formula (A-II).

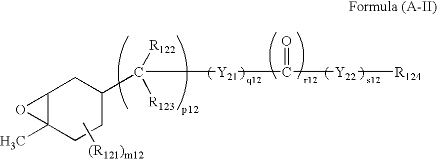

In above Formula (A-II), R₁₂₁ is identical to aforesaid R₁₁₁. Y₂₁ and Y₂₂ are each O or S, but preferably O. “m12” is 0-2, but preferably 0 or 1. “p12” is 0, 1 or 2; q12, r12 and s12 are 0 or 1, and R₁₂₂, R₁₂₃ and R₁₂₄ are identical to R₁₁₂, R₁₁₃ and R₁₁₄.

In Formula (A-II), R₁₂₁ is a substituent group, examples of which include a halogen atom (such as a chlorine atom, a bromine atom, and a fluorine atom); an alkyl group having a carbon number of 1-20 (such as a methyl group, an ethyl group, an isopropyl group, and a butyl group); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferable substituent groups are an alkoxy group and an alkoxycarbonyl group.

Y₂₁ and Y₂₂ are each O or S, but preferably O. “m12” is 0-2, but preferably 0 or 1. “p12” is 0, 1 or 2, and q12, r12, and s12 are 0 or 1.

R₁₂₂, R₁₂₃ and R₁₂₄ are each a hydrogen atom or a substituted or unsubstituted alkyl group. Examples of an alkyl group include groups identical to examples of the alkyl group of R₁₁₁ described above. Examples of a substituent group of an alkyl group incorporating a substituent group include a halogen atom (such as a chlorine atom, a bromine atom and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferable substituent groups are the alkoxy group and the alkoxycarbonyl group.

Further preferable alicyclic epoxides, with respect to forming a cured film exhibiting excellent hardness, enhanced adhesion of the cured film to the substrate, and curing sensitivity barely affected by variations in the printing environment, are compounds represented by following Formulas (A-III), (A-IV) or (A-V).

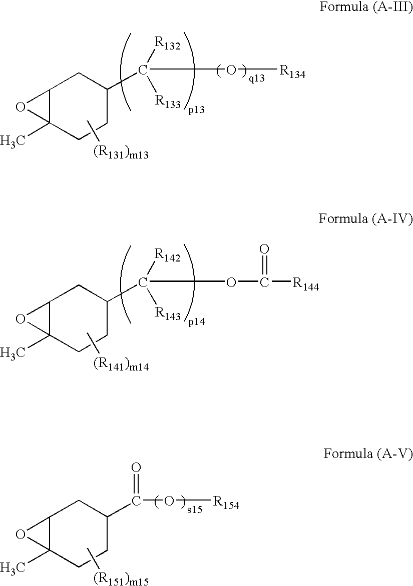

In the above formulas, R₁₃₁, R₁₄₁ and R₁₅₁ are identical to aforesaid R₁₁₁. “m13”, “m14” and “m15” are each 0-2, but preferably 0 or 1. “p13” and “p14” are each 0, 1 or 2; and “q13” and “s15” are each 0 or 1. R₁₃₂, R₁₃₃, R₁₃₄, R₁₄₂, R₁₄₃, R₁₄₄ and R₁₅₄ are identical to R₁₁₂, R₁₁₃ and R₁₁₄.

In Formula (A-III), (A-IV) or (A-V), R₁₃₁, R₁₄₁ and R₁₅₁ are each a substituent group, examples of which include a hydrogen atom or a substituted or unsubstituted alkyl group.

Examples of the alkyl group include groups identical to examples of the alkyl group of R₁₁₁ described above. Examples of a substituent group of an alkyl group having a substituent group include a halogen atom (such as a chlorine atom, a bromine atom and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); and an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferred substituent groups are the alkoxy group and the alkoxycarbonyl group.

“m13”, “m14”, and “m15” are each 0-2, “p13” and “p14” are each 0, 1, or 2, and “q13” and “q15” are each 0 or 1.

R₁₃₂, R₁₃₃, R₁₃₄, R₁₄₂, R₁₄₃, R₁₄₄, and R₁₅₄ are a hydrogen atom or a substituted or unsubstituted alkyl group. Examples of an alkyl group include groups identical to examples of the alkyl group of R₁₁₁ described above. Examples of a substituent group of an alkyl group incorporating a substituent group include a halogen atom (such as a chlorine atom, a bromine atom and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); and an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferred substituent groups are the alkoxy group and the alkoxycarbonyl group.

Specifically preferred alicyclic epoxide, with respect to forming cured film of excellent hardness, enhanced adhesion of cured film to the substrate, and curing sensitivity barely affected by variations in printing environment, is a compound represented by aforesaid Formula (A-VI).

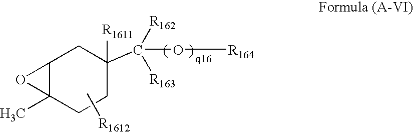

In the above formula, R₁₆₁₁ and R₁₆₁₂ are each a hydrogen atom or an alkyl group having a carbon number of 1-6 (such as a methyl group, an ethyl group, a propyl group, an isopropyl group, a butyl group, a sec-butyl group, t-butyl group, a pentyl group and a hexyl group); and preferred alkyl groups are a methyl group, an ethyl group and a propyl group. R₁₆₂, R₁₆₃ and R₁₆₄ are identical to R₁₁₂, R₁₁₃ and R₁₁₄. “q16” is 0 or 1.

In Formula (A-VI), R₁₆₁₁ and R₁₆₁₂ are each a hydrogen atom or an alkyl group having a carbon number of 1-6 (such as a methyl group, an ethyl group, a propyl group, an isopropyl group, a butyl group, a sec-butyl group, t-butyl group, a pentyl group and a hexyl group); and preferable alkyl groups are a methyl group, an ethyl group and a propyl group. R₁₆₂, R₁₆₃ and R₁₆₄ are identical to R₁₁₂, R₁₁₃ and R₁₁₄.

R₁₆₂, R₁₆₃, and R₁₆₄ are each independently a hydrogen atom or a substituted or an unsubstituted alkyl group. Examples of the alkyl group include groups identical to examples of an alkyl group of R₁₁₁ described earlier. Examples of a substituent group of an alkyl group having a substituent group include a halogen atom (such as a chlorine atom, a bromine atom and a fluorine atom); an alkoxy group having a carbon number of 1-20 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group having a carbon number of 1-20 (such as an acetoxy group, a propionyloxy group and a trifluoroacetoxy group); an alkoxycarbonyl group having a carbon number of 1-20 (such as a methoxycarbonyl group, an ethoxycarbonyl group and a tert-butoxycarbonyl group); and an alkylthiocarbonyl group having a carbon number of 2-20 (such as a methylthiocarbonyl group, an ethylthiocarbonyl group and a tert-butylthiocarbonyl group); an aryloxycarbonyl group; an alkylsulfonyl group; an arylsulfonyl group; a cyano group; and a nitro group. Preferred substituent groups are the alkoxy group and the alkoxycarbonyl group. “q16” is 0 or 1.

In the following, specific examples of Formula (A) of this invention will be cited, however, this invention is not limited thereto.

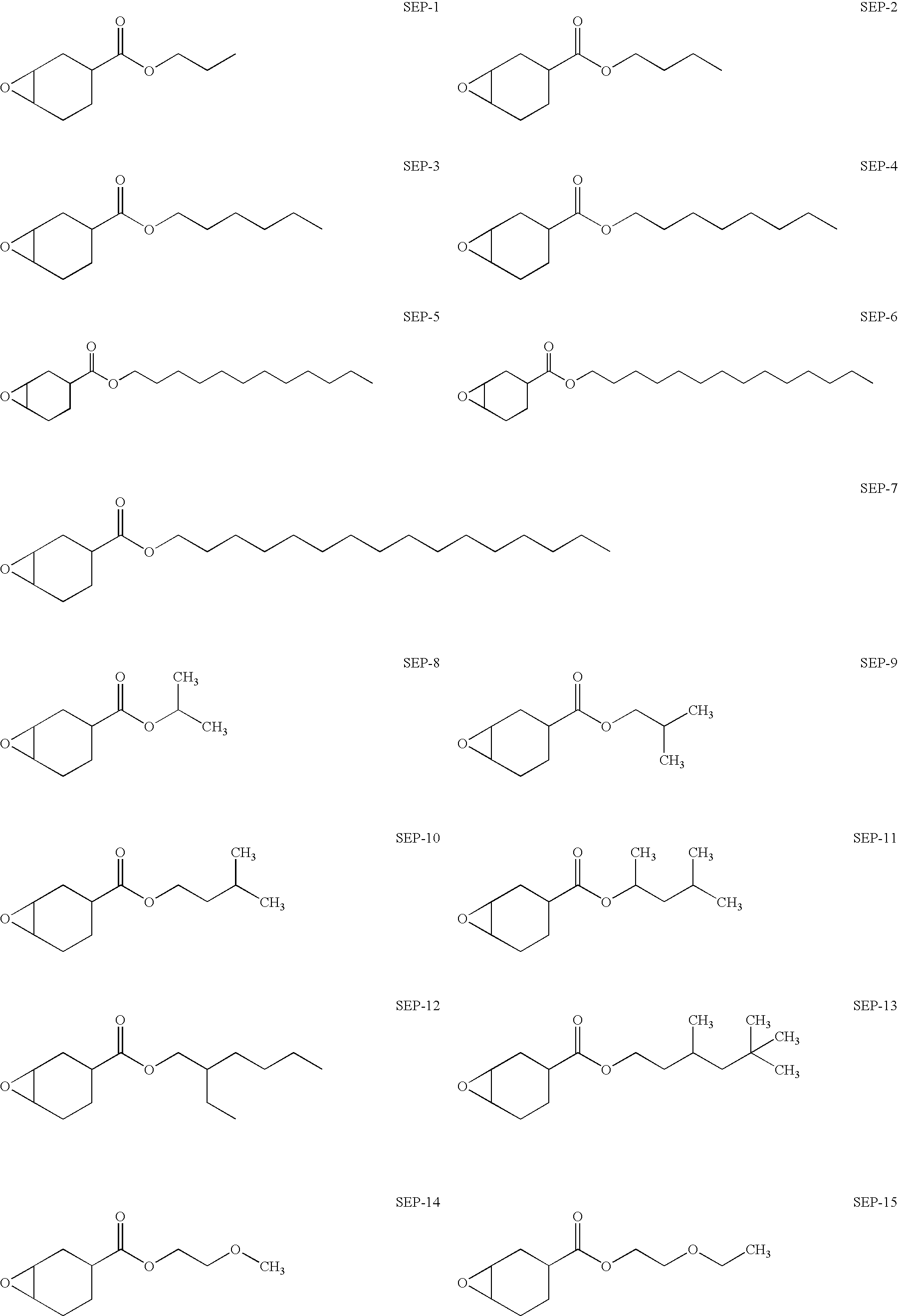

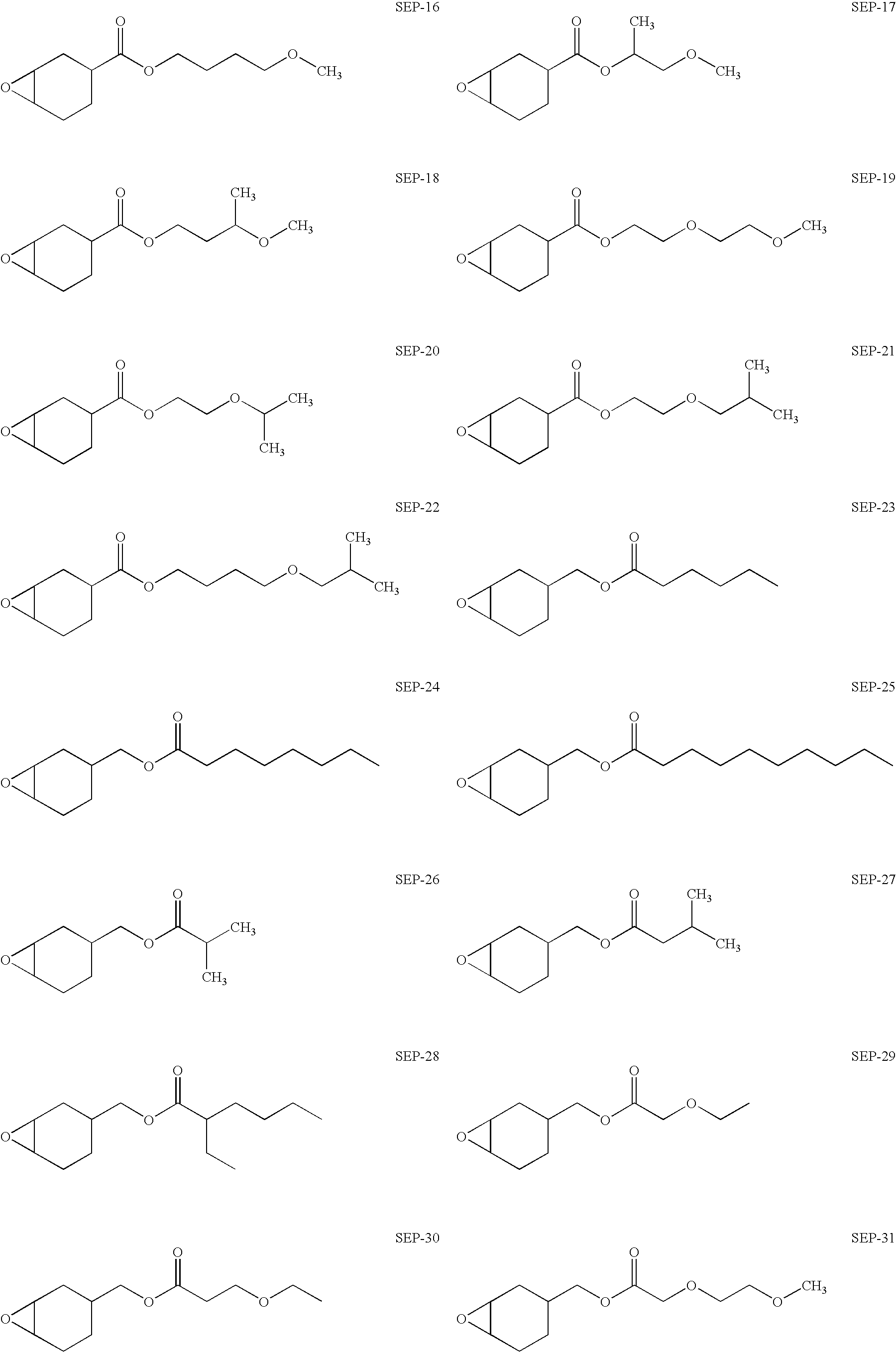

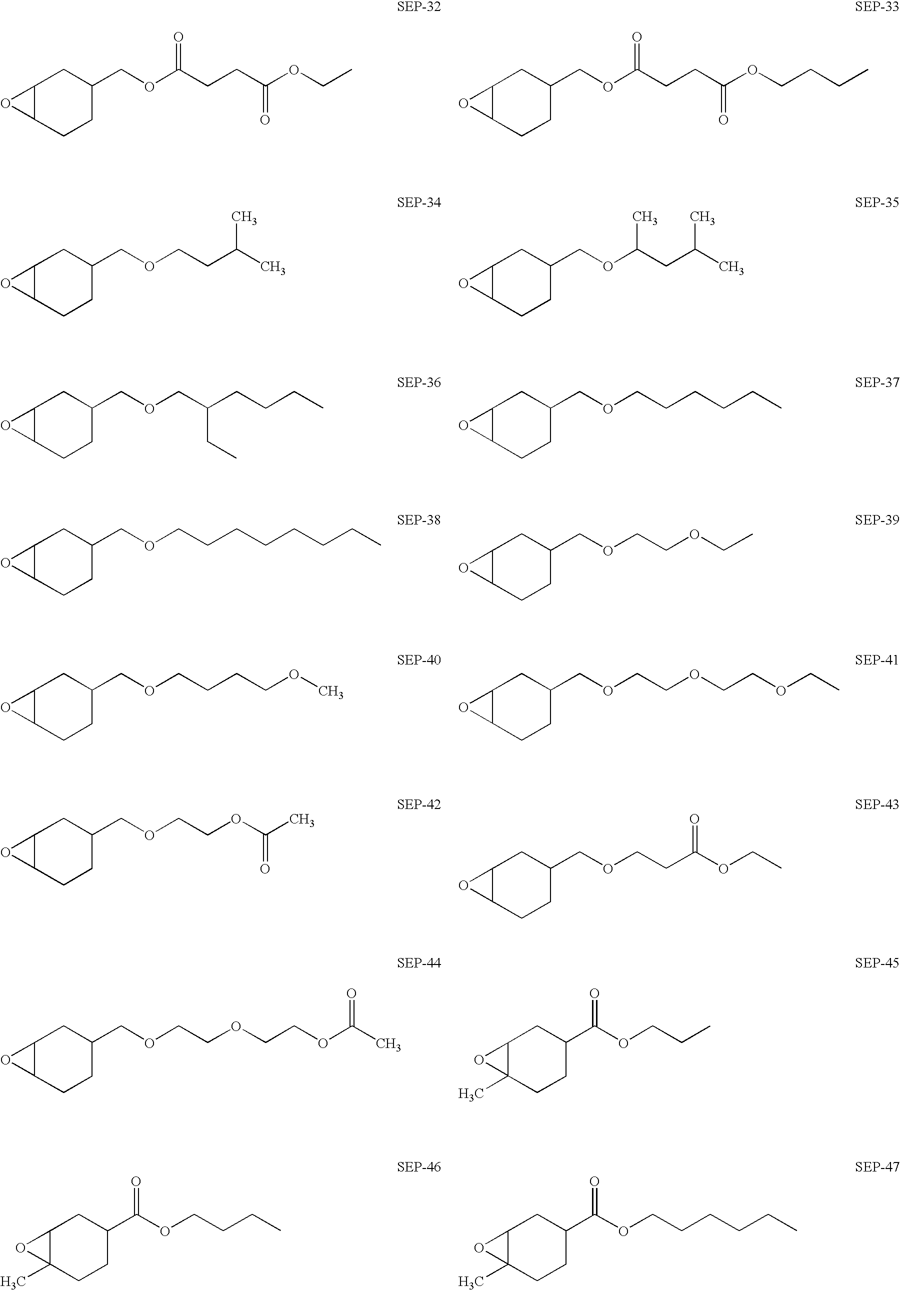

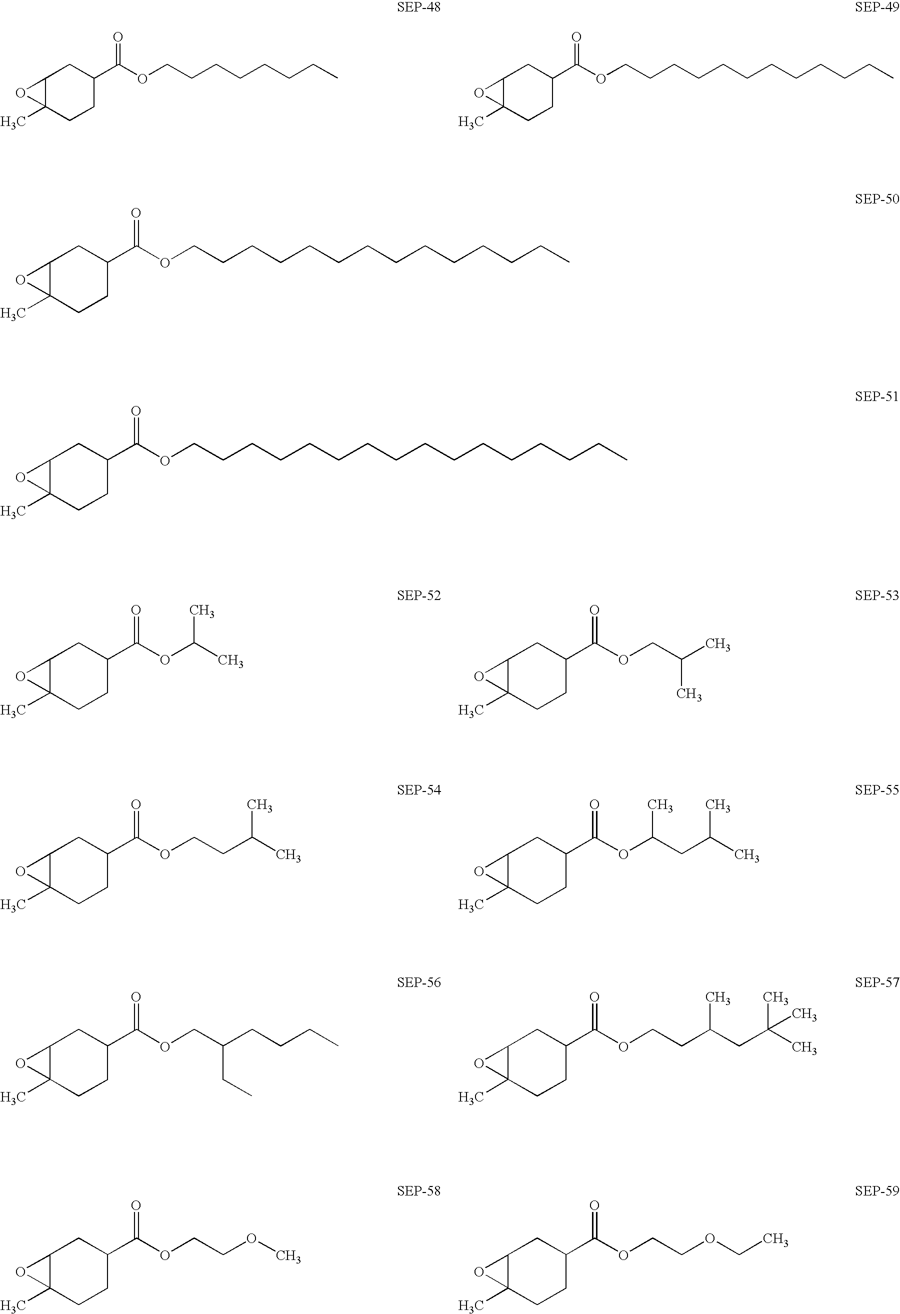

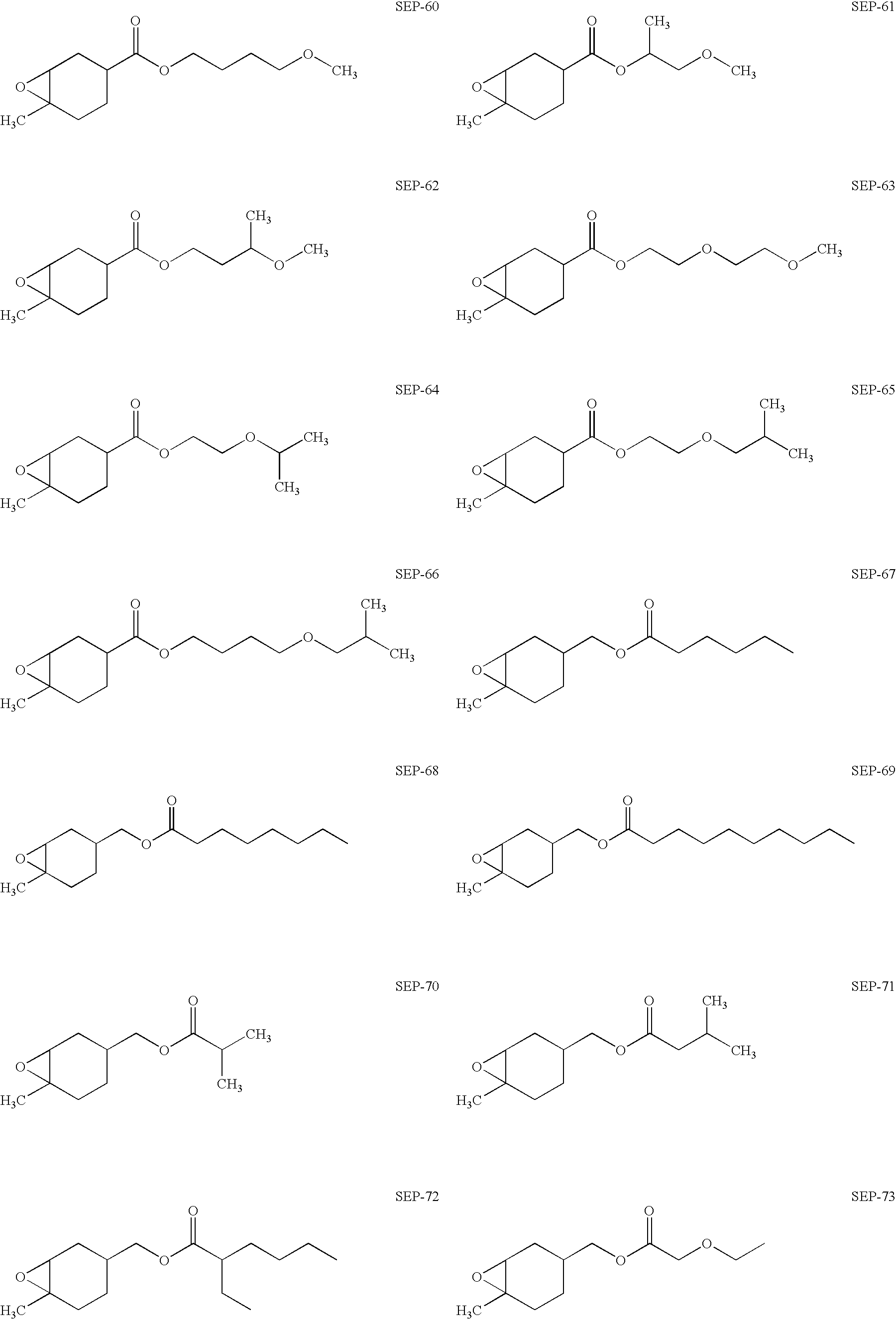

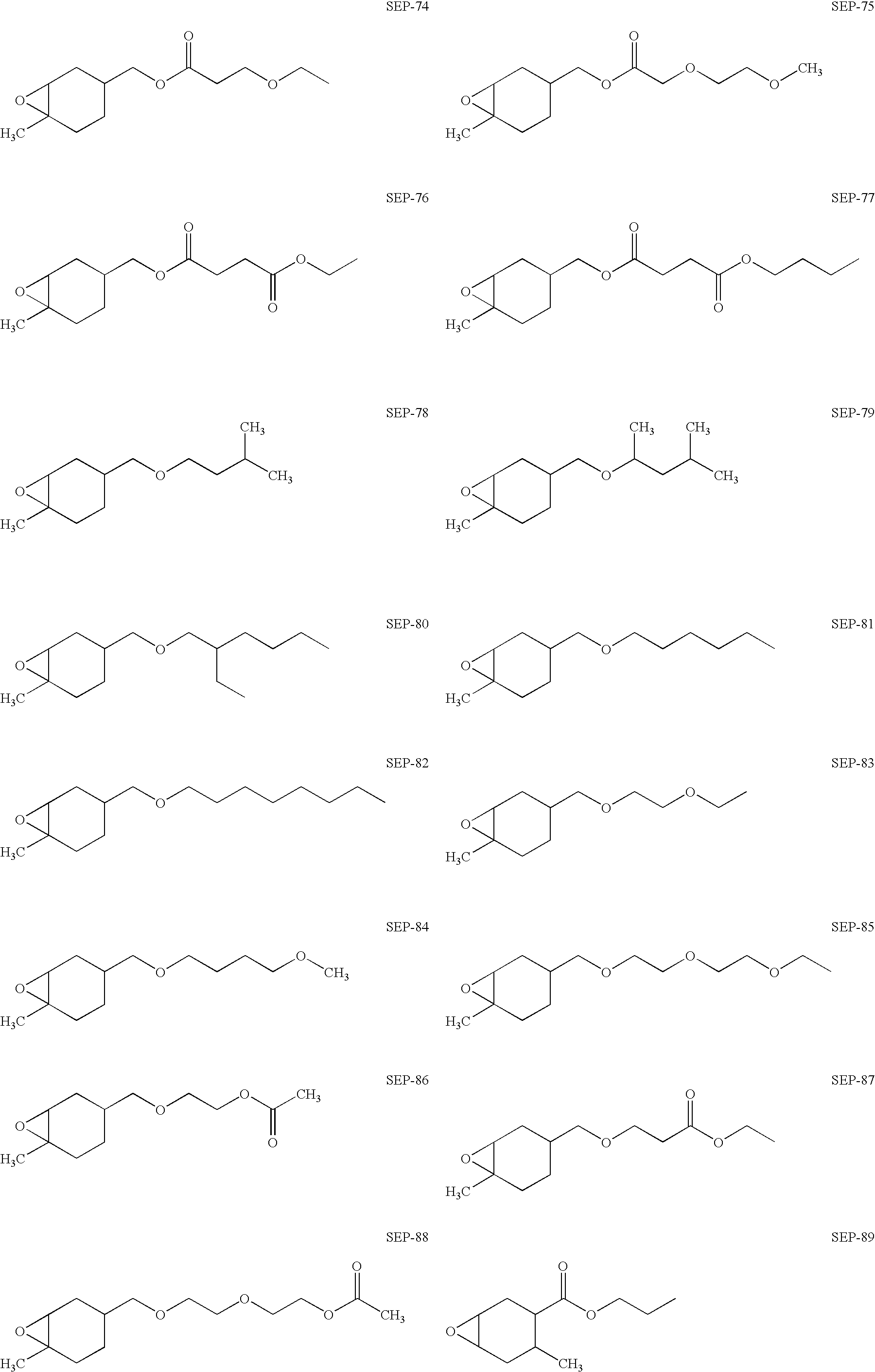

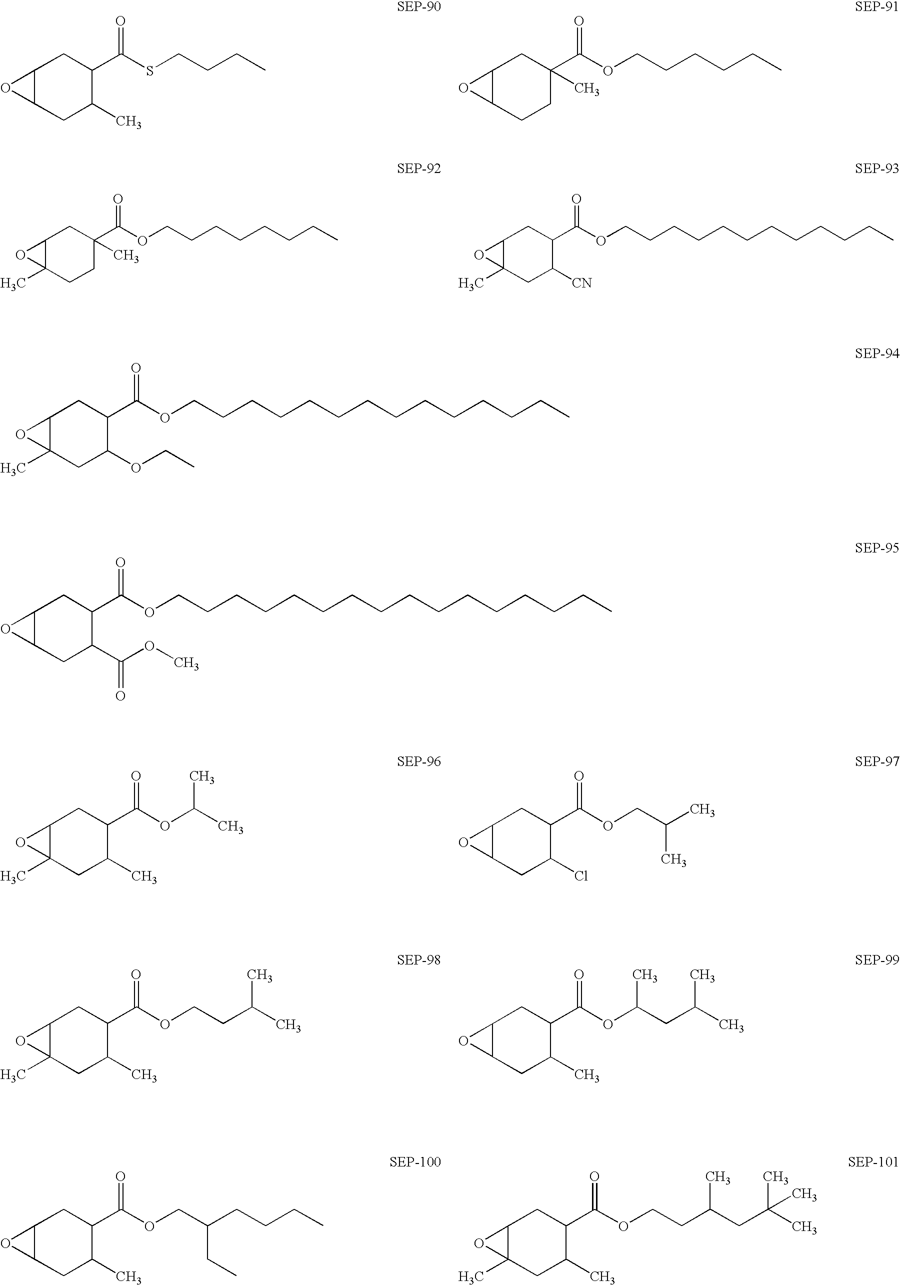

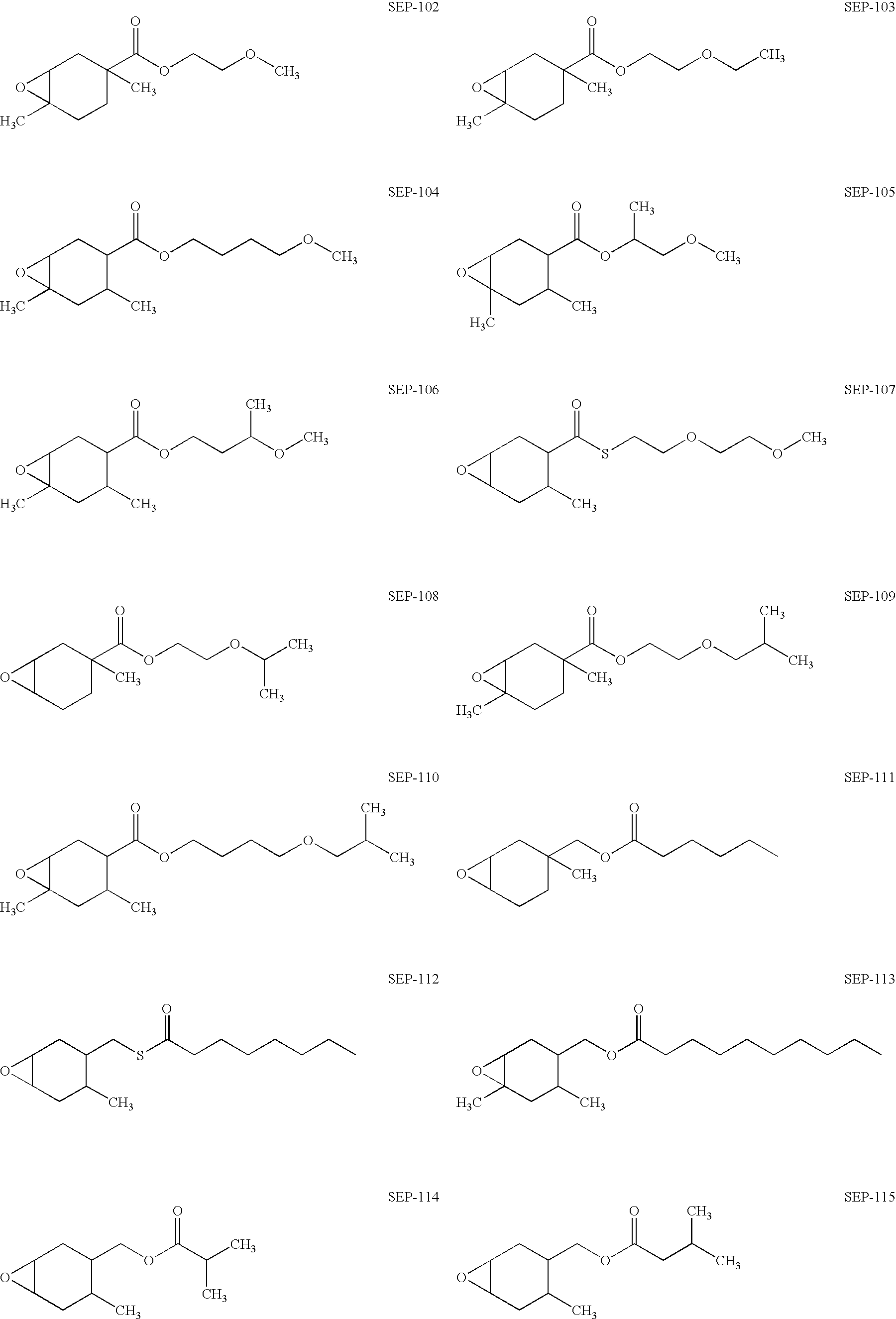

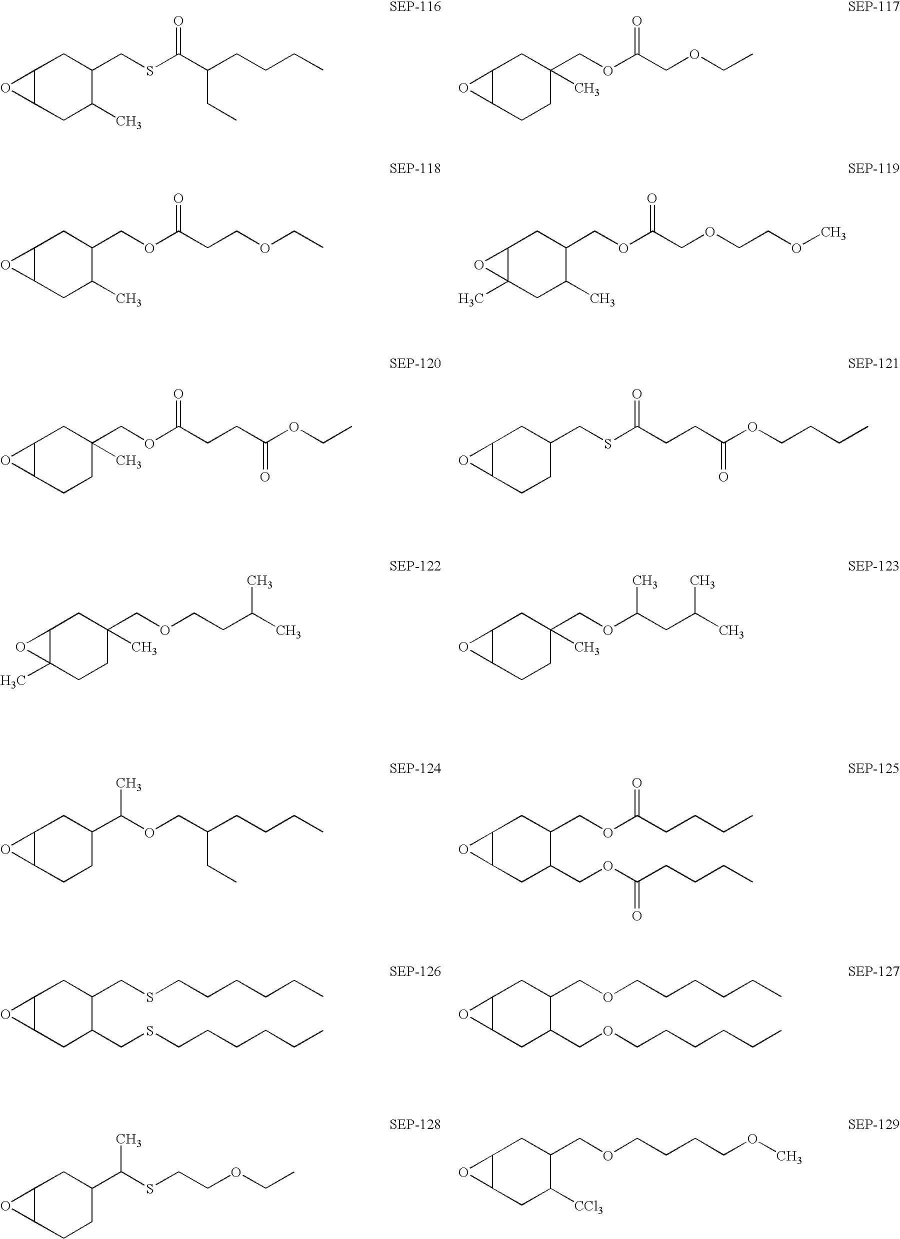

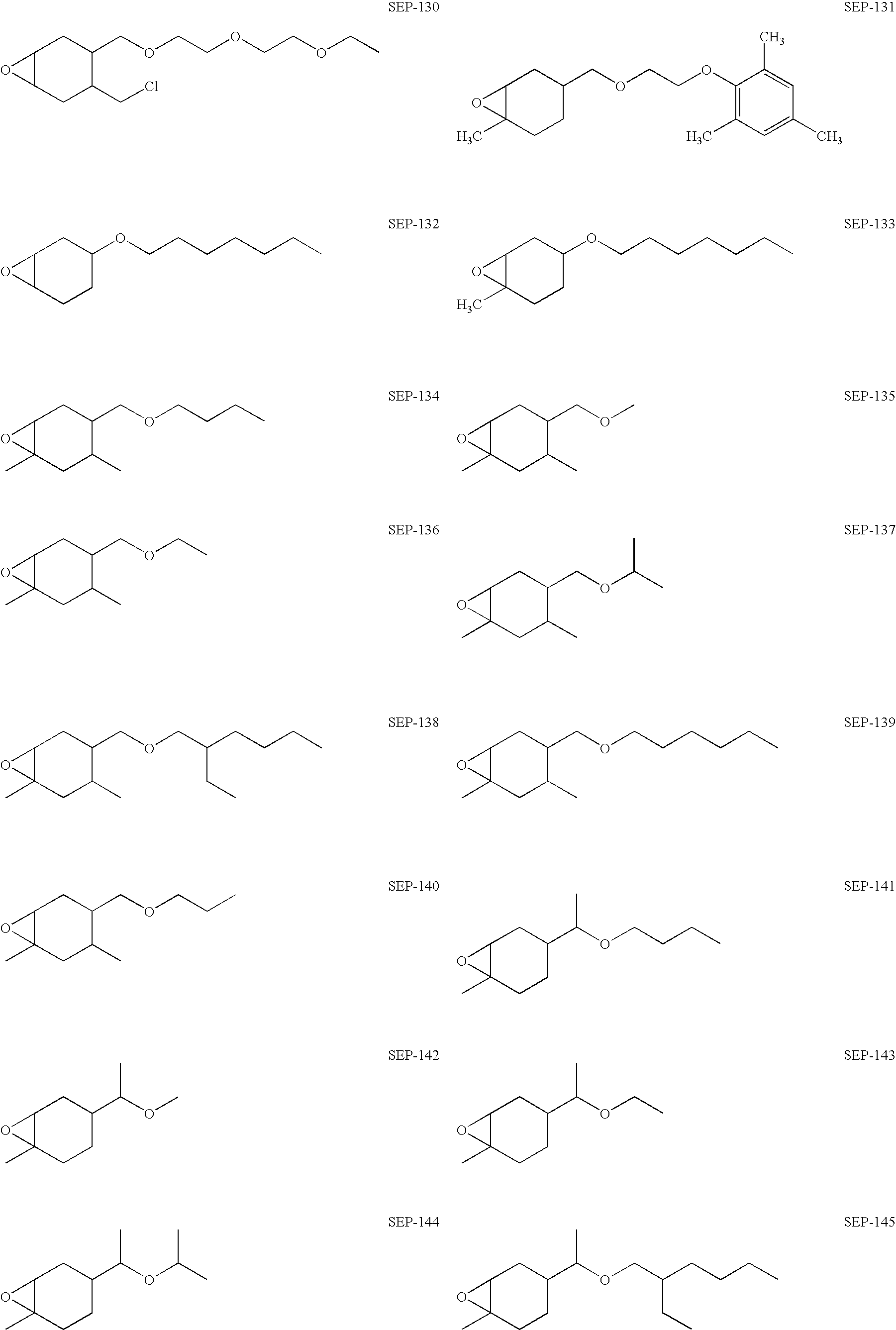

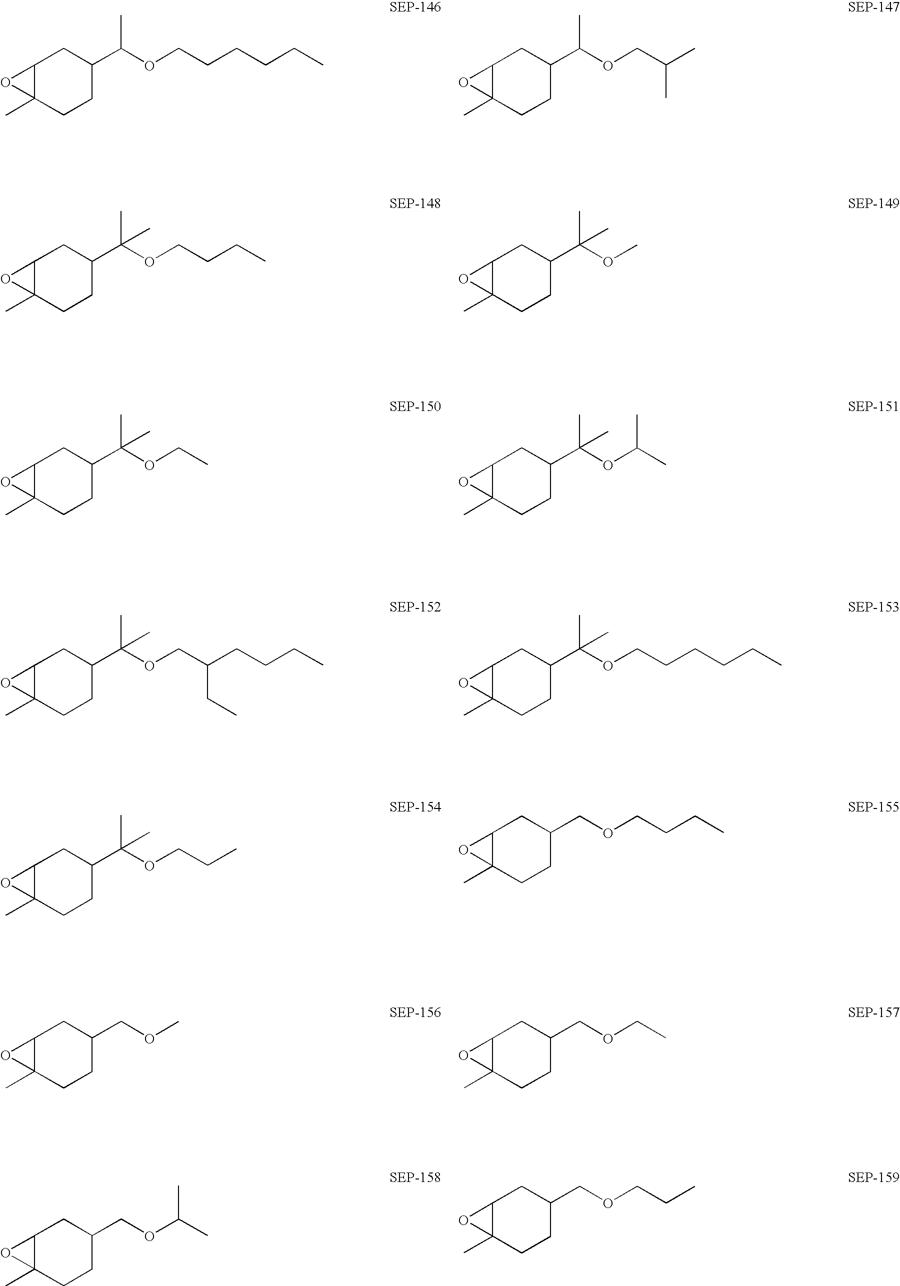

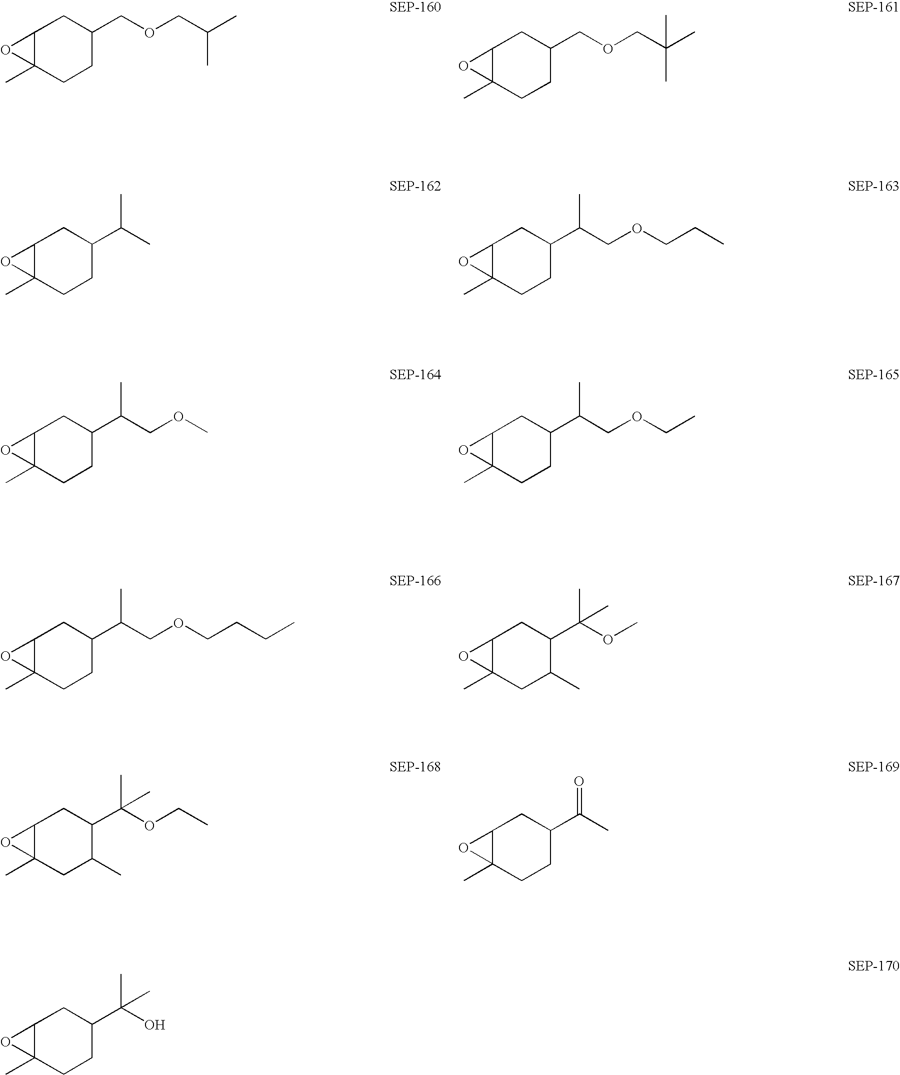

The added amount of a mono-functional epoxy compound of this invention is preferably 10-20 weight %, with respect to providing flexibility to the cured film and other physical properties of the film after curing. In this invention, one type of a mono-functional epoxy compound may be utilized alone or at least two types may be utilized in appropriate combinations.

These alicyclic epoxy compounds, although the manufacturing method is not specifically limited, can be synthesized with reference to, for example, the fourth edition, Experimental Chemistry Course 20, Organic Synthesis II, p. 213-, published by Maruzen Co. Ltd. (1992); “The Chemistry of Heterocyclic Compounds—Small Ring Heterocycles part 3, Oxiranes”, edited by Alfred Hasfner, John & Willey and Sons, An Interscience Publication, New York (1985); Yoshimura, Adhesion vol. 29, No. 12, 32 (1985), Yoshimura, Adhesion vol. 30, No. 5, 42 (1986), Yoshimura, Adhesion vol. 30, No. 7, 42 (1986), JP-A Nos. 11-100378, 4-36263 and 4-69360.

In the active ray curable compositions of this invention, a poly-functional alicyclic epoxy compound may be utilized in combination together with a compound represented by Formula (X), an oxetane compound, and a mono-functional epoxy compound represented by Formula (A), whereby, improved sensitivity and enhanced cured film properties can be obtained.

As a bi-functional alicyclic epoxide, preferable is a poly-functional epoxide compound represented by following Formula (B).

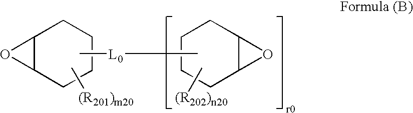

In the above formula, R₂₀₁ and R₂₀₂ are each a substituent group, and m20 and n20 are each 0, 1 or 2, but preferably 0 or 1. “r0” is 1-3. L₀ is an r0+1 valent connecting group, which may contain an oxygen atom or a sulfur atom in the main chain, and exhibits a carbon number of 1-15, or a single bond.

In the above formula, examples of the substituent group represented by R₂₀₁ and R₂₀₂ are a halogen atom (such as a chlorine atom, a bromine atom and a fluorine atom); an alkyl group having a carbon number of 1-6 (such as a methyl group, an ethyl group, a propyl group, an isopropyl group and a butyl group); an alkoxy group having a carbon number of 1-6 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group (such as an acetoxy group, a propionyloxy group and a trifluoroacetoxy group); an alkoxycarbonyl group (such as a methoxycarbonyl group, an ethoxycarbonyl group and a tert-butoxycarbonyl group). Preferred substituent groups are the alkyl group, the alkoxy group and the alkoxycarbonyl group.

Examples of a divalent connecting group, which may contain an oxygen atom or a sulfur atom in the main chain and has a carbon number of 1-15, includes the following groups and groups formed by combining these groups with a plural number of an —O— group, an —S— group, a —CO— group and a —CS— group.
Methylene group [—CH₂—]
Ethylidene group [>CHCH₃]
Isopropylidene group [>C(CH₃)₂]
1,2-Ethylene group [—CH₂CH₂—]
1,2-Propylene group [—CH(CH₃)CH₂—]
1,3-Propanediyl group [—CH₂CH₂CH₂—]
2,2-Dimethyl-1,3-propanediyl group [—CH₂C(CH₃)₂CH₂—]
2,2-Dimethoxy-1,3-propanediyl group [—CH₂C(OCH₃)₂CH₂—]
2,2-Dimethoxymethyl-1,3-propanediyl group [—CH₂C(CH₂OCH₃)₂CH₂—]
1-Methyl-1,3-propanediyl group [—CH(CH₃)CH₂CH₂—]
1,4-Butanediyl group [—CH₂CH₂CH₂CH₂—]
1,5-Pentanediyl group [—CH₂CH₂CH₂CH₂CH₂—]
Oxydiethylene group [—CH₂CH₂OCH₉CH₂—]
Thiodiethylene group [—CH₂CH₂SCH₂CH₂—]
3-Oxthothiodiethylene group [—CH₂CH₂SOCH₂CH₂—]
3,3-Dioxthothiodiethylene group [—CH₂CH₂SO₂CH₂CH₂—]
1,4-Dimethyl-3-oxa-1,5-pentadiyl group [—CH(CH₃)CH₂OCH(CH₃)CH₂—]
3-Oxopentanediyl group [—CH₂CH₂COCH₂CH₂—]
1,5-Dioxo-3-oxapentanediyl group [—COCH_OCH₂CO—]
4-Oxa-1,7-heptanediyl group [—CH₂CH₂CH₂OCH₂CH₂CH₂—]
3,6-Dioxa-1,8-octanediyl group [—CH₂CH₂OCH₂CH₂OCH₂CH₂—]
1,4,7-Trimethyl-3,6-dioxa-1,8-octanediyl group [—CH(CH₃)CH₂O—CH(CH₃)CH₂OCH(CH₃)CH₂—]
5,5-Dimethyl-3,7-dioxa-1,9-nonanediyl group [—CH₂CH₂OCH₂C(CH₃)₂CH₂OCH₂CH₂—]
5,5-Dimethoxy-3,7-dioxa-1,9-nonanediyl group [—CH₂CH₂OCH₂C(OCH₃)₂CH₂OCH₂CH₂—]
5,5-Dimethoxymethyl-3,7-dioxa-1,9-nonanediyl group [—CH₂CH₂OCH₂C(CH₂OCH₃)₂CH₂OCH₂CH₂—]
4,7-Dioxo-3,8-dioxa-1,10-decanediyl group [—CH₂CH₂OCOCH₂CH₂COOCH₂CH₂—]
3,8-Dioxo-4,7-dioxa-1,10-decanediyl group [—CH₂CH₂COOCH₂CH₂OCOCH₂CH₂—]
1,3-Cyclopentanediyl group [-1,3-C₅H₈—]
1,2-Cyclohexanediyl group [-1,2-C₆H₁₀—]
1,3-Cyclohexanediyl group [-1,3-C₆H₁₀—]
1,4-Cyclohexanediyl group [-1,4-C₆H₁₀—]
2,5-Tetrahydrofurandiyl group [2,5-C₄H₆O—]
p-Phenylene group [-p-C₆H₄—]
m-Phenylene group [-m-C₆H₄—]
α,α′-o-Xylylene group [-o-CH₂—C₆H₄—CH₂—]
α,α′-m-Xylylene group [-m-CH₂—C₆H₄—CH₂—]
α,α′-p-Xylylene group [-p-CH₂—C₆H₄—CH₂—]
Furan-2,5-diyl-bismethylene group [2,5-CH₂—C₄H₂O—CH₂—]
Thiophen-2,5-diyl-bismethylene [2,5-CH₂—C₄H₂S—CH₂—]
i-Propylidenbis-p-phenylene [-p-C₆H₄—C(CH₃)₂-p-C₆H₄—]

A not less than tri-valent connecting group includes groups which are formed by removing necessary hydrogen atoms at appropriate positions from the divalent groups listed above, and groups formed by combining them with a plural number of an —O— group, an —S— group, a —CO— group and a —CS— group.

L₀ may be provided with a substituent group. Examples of such a substituent group include a halogen atom (such as a chlorine atom, a bromine atom and a fluorine atom); an alkyl group having a carbon number of 1-6 (such as a methyl group, an ethyl group, a propyl group, an isopropyl group and a butyl group); an alkoxy group having a carbon number of 1-6 (such as a methoxy group, an ethoxy group, an n-propoxy group, an iso-propoxy group, an n-butoxy group and a tert-butoxy group); an acyl group (such as an acetyl group, a propionyl group and a trifluoroacetyl group); an acyloxy group (such as an acetoxy group, a propionyloxy group and a trifluoroacetoxy group); and an alkoxycarbonyl group (such as a methoxycarbonyl group, an ethoxycarbonyl group and a tert-butoxycarbonyl group). A preferable substituent is the alkyl group, the alkoxy group or the alkoxycarbonyl group.

L₀ is preferably a divalent connecting group which may contain an oxygen atom or a sulfur atom in the main chain and having a carbon number of 1-8, but more preferable is a divalent connecting group, the main chain of which is comprised of only carbon atoms and having a carbon number of 1-5.

Specifically preferable alicyclic epoxide, with respect to forming cured film exhibit excellent hardness and enhanced adhesion of cured film to the substrate, is a compound represented by following Formula (B-I) or (B-II).

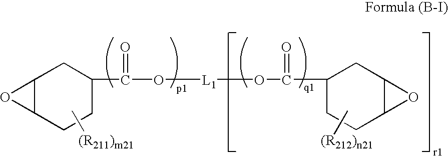

In the above formula, R₂₁₁ and R₂₁₂ are a substituent group, “m21” and “n21” are 0, 1 or 2, but preferably 0 or 1. “p1” and “q1” are each 0 or 1. “r1” is 1-3. L₁ is an r1+1 valent connecting group, which may contain an oxygen atom or a sulfur atom in the main chain and has a carbon number of 1-15, or a single bond.

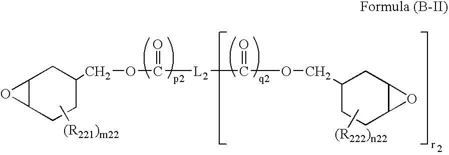

In this formula, R₂₂₁ and R₂₂₂ are a substituent group, m22 and n22 are 0, 1 or 2, but preferably 0 or 1. “p2” and “q2” are each 0 or 1, and “r2” is 1-3. L₇ is an r2+1 valent connecting group, which may contain an oxygen atom or a sulfur atom in the main chain and has a carbon number of 1-15, or a single bond.

In the above formula, R₂₁₁, R₂₁₂, R₂₂₁ and R₂₂₂ are identical to R₁₁₁ in aforesaid Formula (A-1).

Examples of a divalent connecting group, represented by L₁ and L₂, which may contain an oxygen atom or a sulfur atom in the main chain and has a carbon number of 1-15, include those described in the explanation of L₀. L₁ and L₂ are preferably a divalent connecting group which may contain an oxygen atom or a sulfur atom in the main chain and having a carbon number of 1-8, but more preferable is a divalent connecting group the main chain of which is comprised of only carbon atoms and having a carbon number of 1-5.

Specifically preferable alicyclic epoxide, with respect to forming cured film exhibiting excellent hardness and curing sensitivity barely affected by variations in printing environment, are compounds represented by following Formula (B-III) or (B-IV).

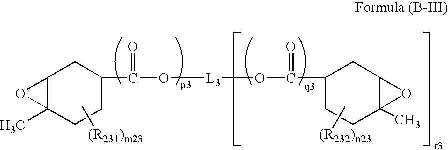

In this formula, R₂₃₁ and R₂₃₂ are a substituent group, while m23 and n23 are 0 or 1. “p3” and “q3” are each 0 or 1, and “r3” is 1-3. L₃ is an r3+1 valent connecting group exhibiting a branched structure, which may contain an oxygen atom or a sulfur atom in the main chain and has a carbon number of 1-15, or a single bond.

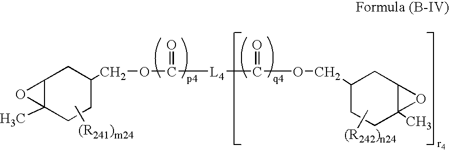

In the above formula, R₂₄₁ and R₂₄₂ are each a substituent group, while m24 and n24 are 0 or 1. “p4” and “q4” are each 0 or 1, and “r4” is 1-3. L₄ is an r4+1 valent connecting group provided with a branched structure, which may contain an oxygen atom or a sulfur atom in the main chain and has a carbon number of 1-15, or a single bond.

In the above formula, R₂₃₁, R₂₃₂, R₂₄₁. and R₂₄₂ are identical to R₁₁₁ in previous Formula (A-1). Examples of a divalent connecting group, represented by L₃ and L₄, which may contain an oxygen atom or a sulfur atom in the main chain and has a carbon number of 1-15, include those described in explanation of L₀.

In the following, specific examples of a preferable alicyclic epoxide will be shown, however, this invention is not limited thereto.

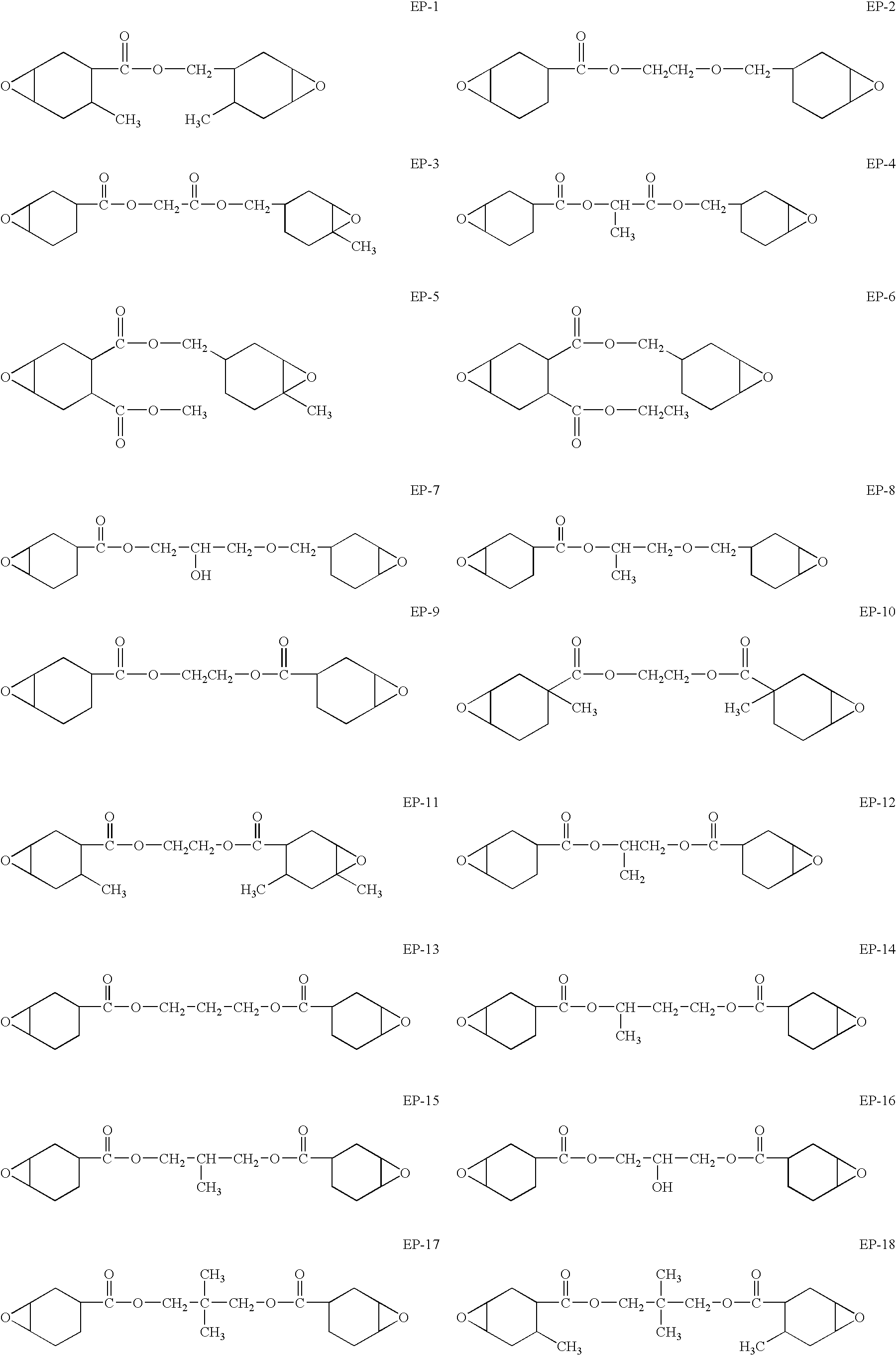

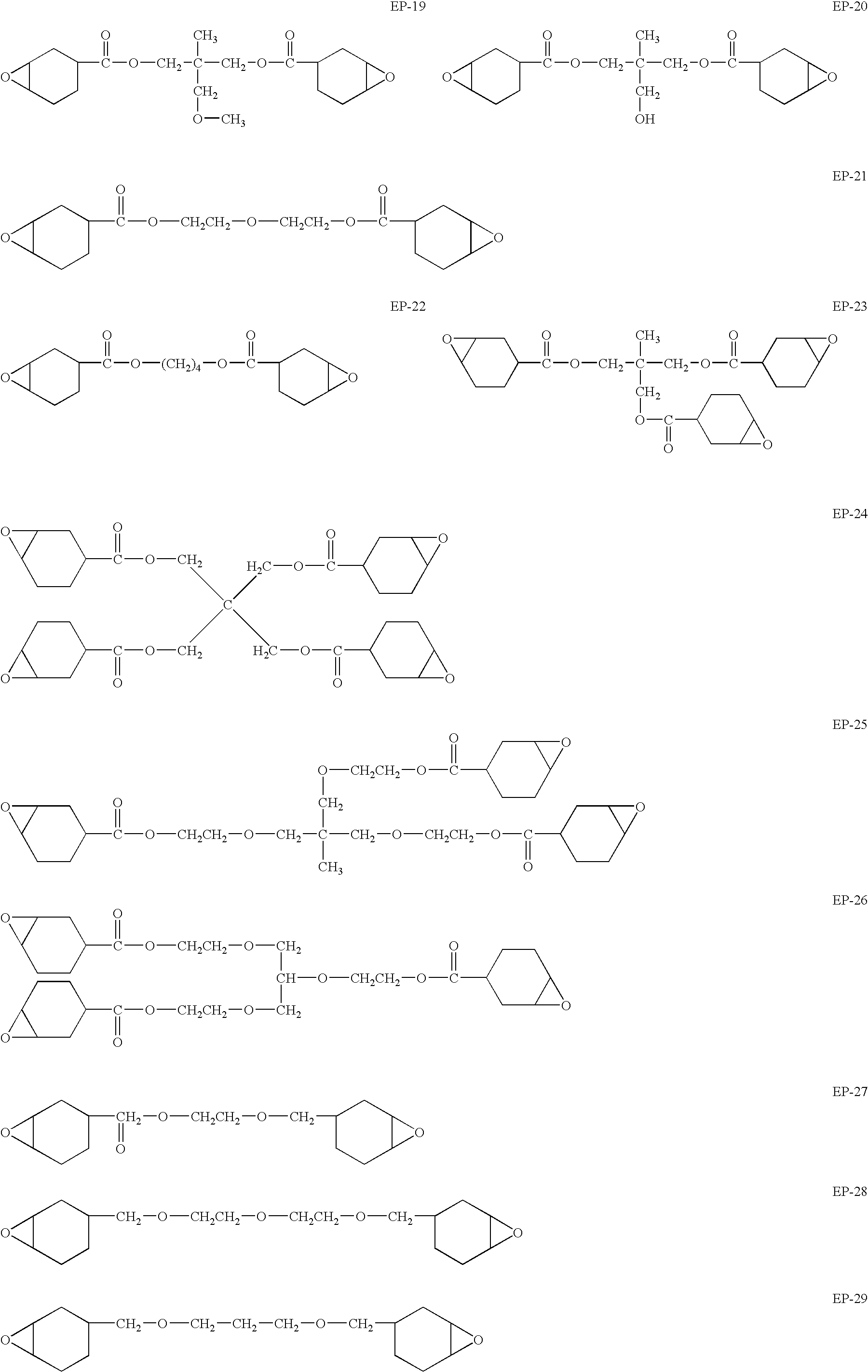

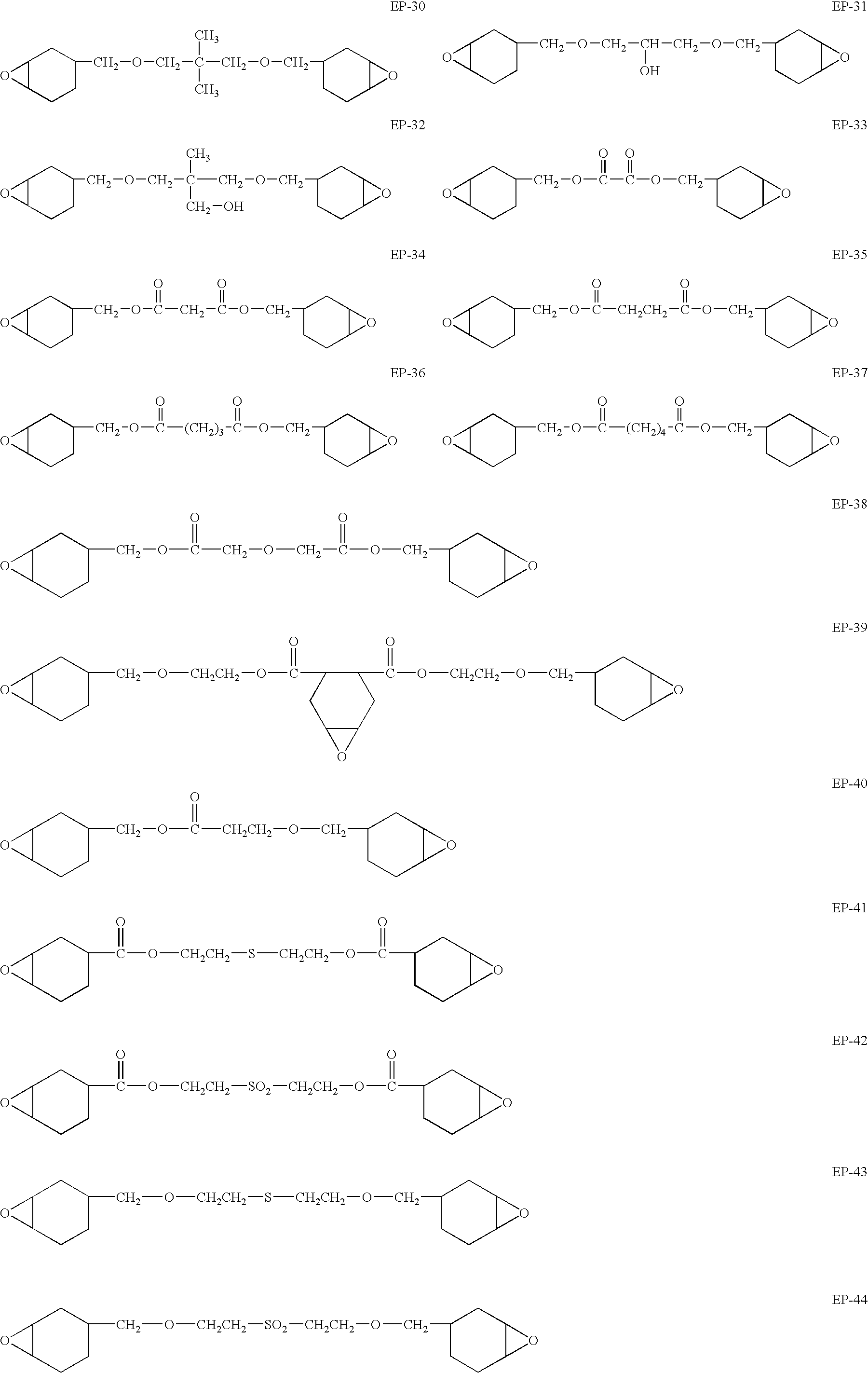

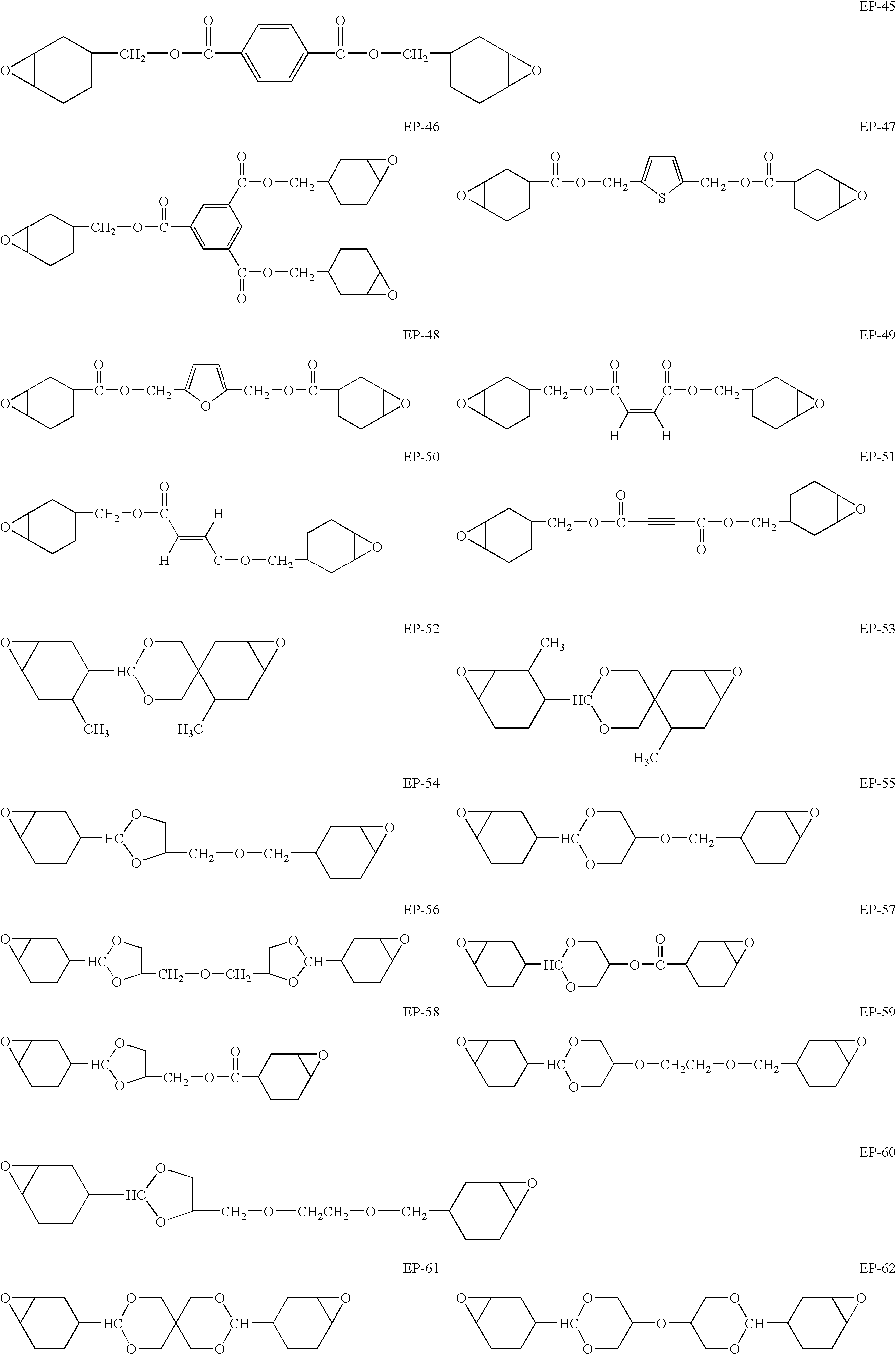

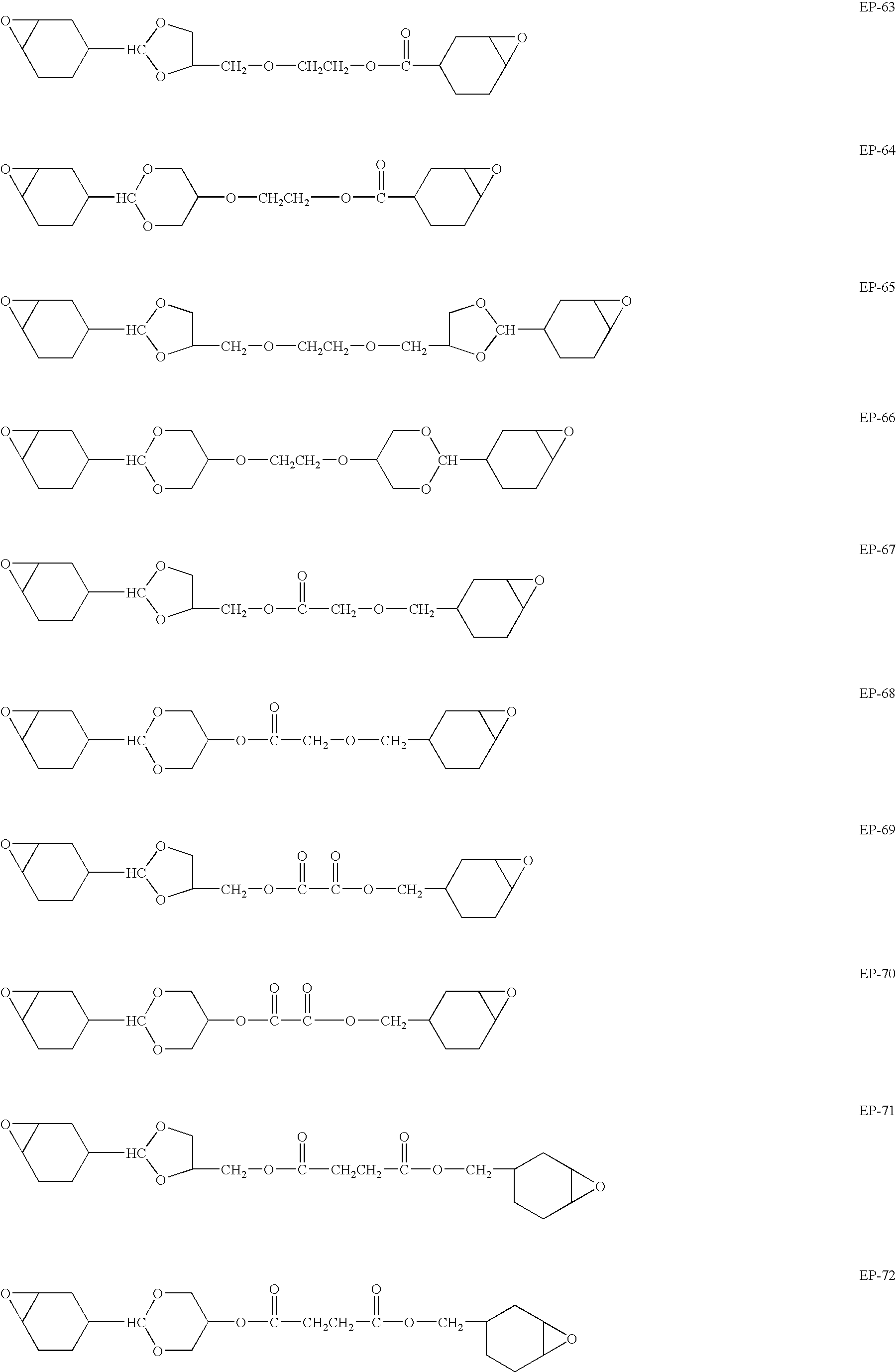

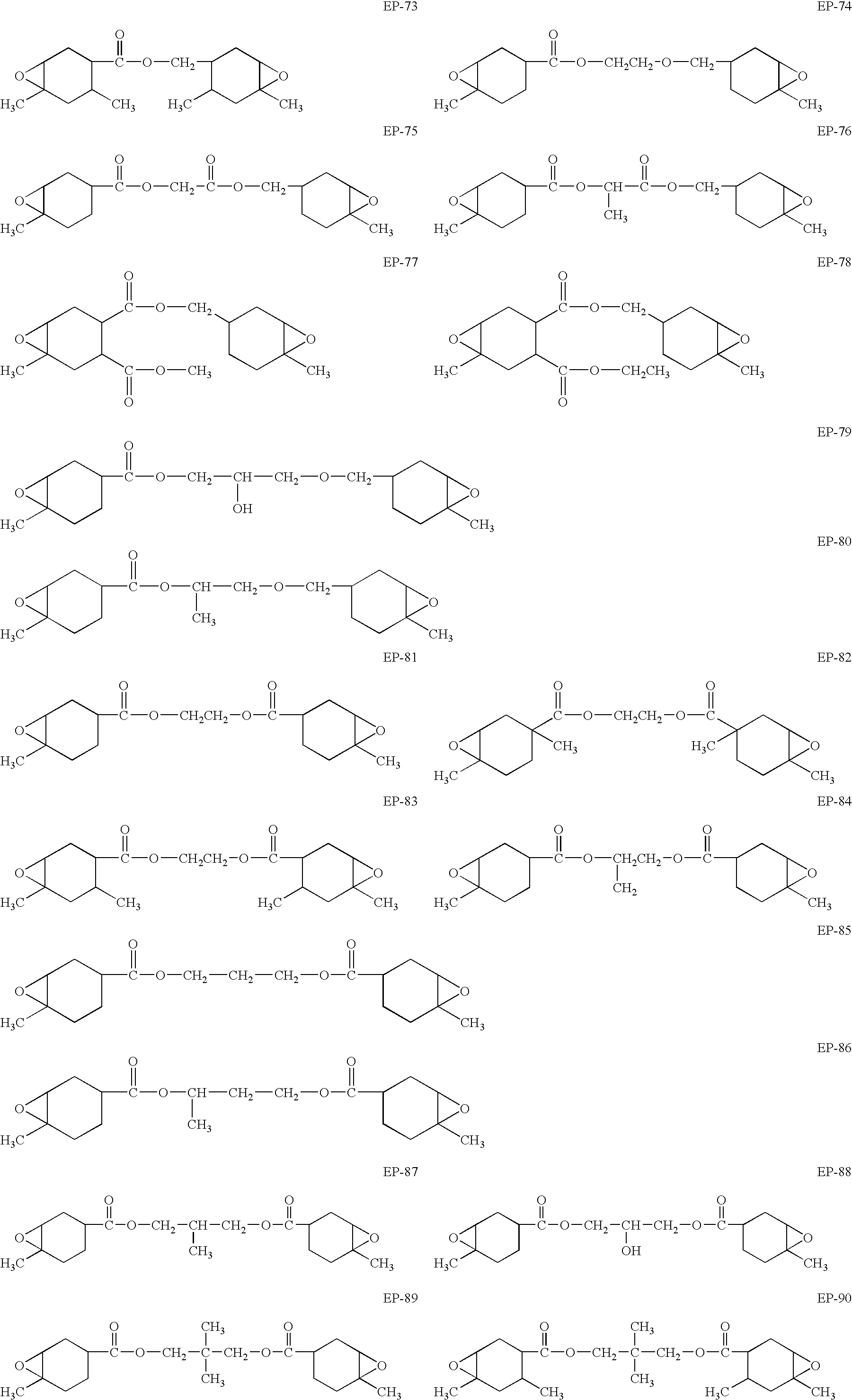

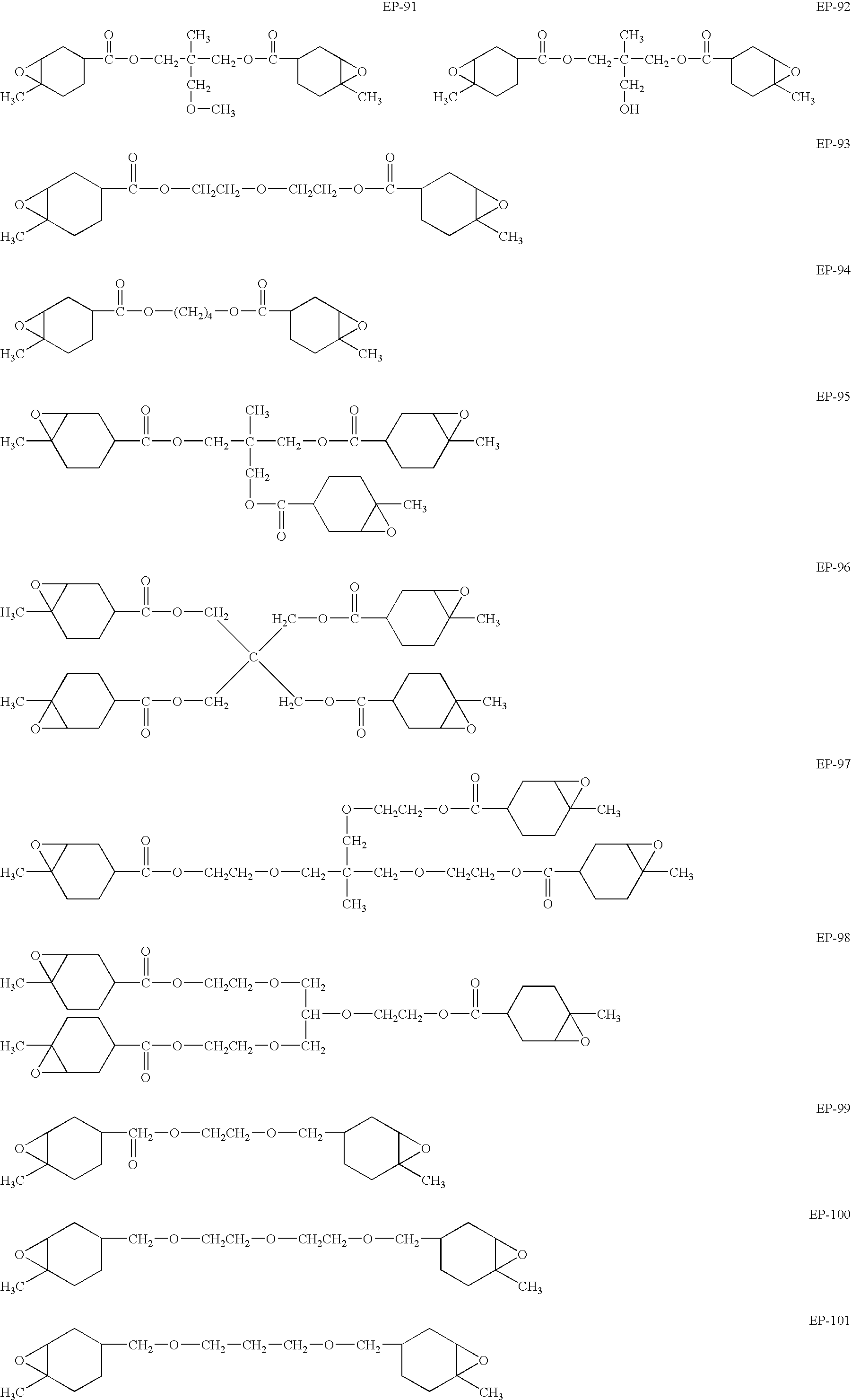

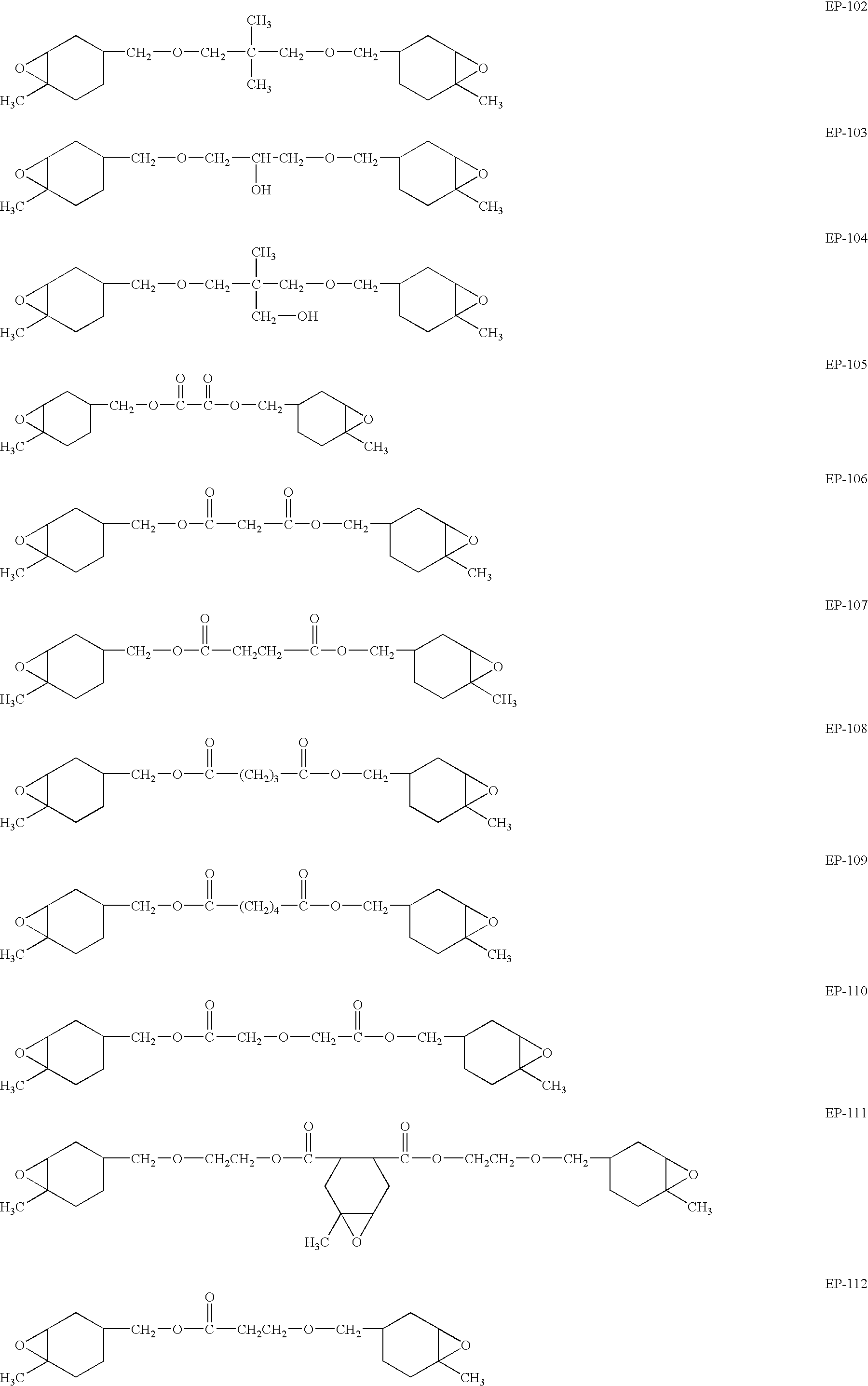

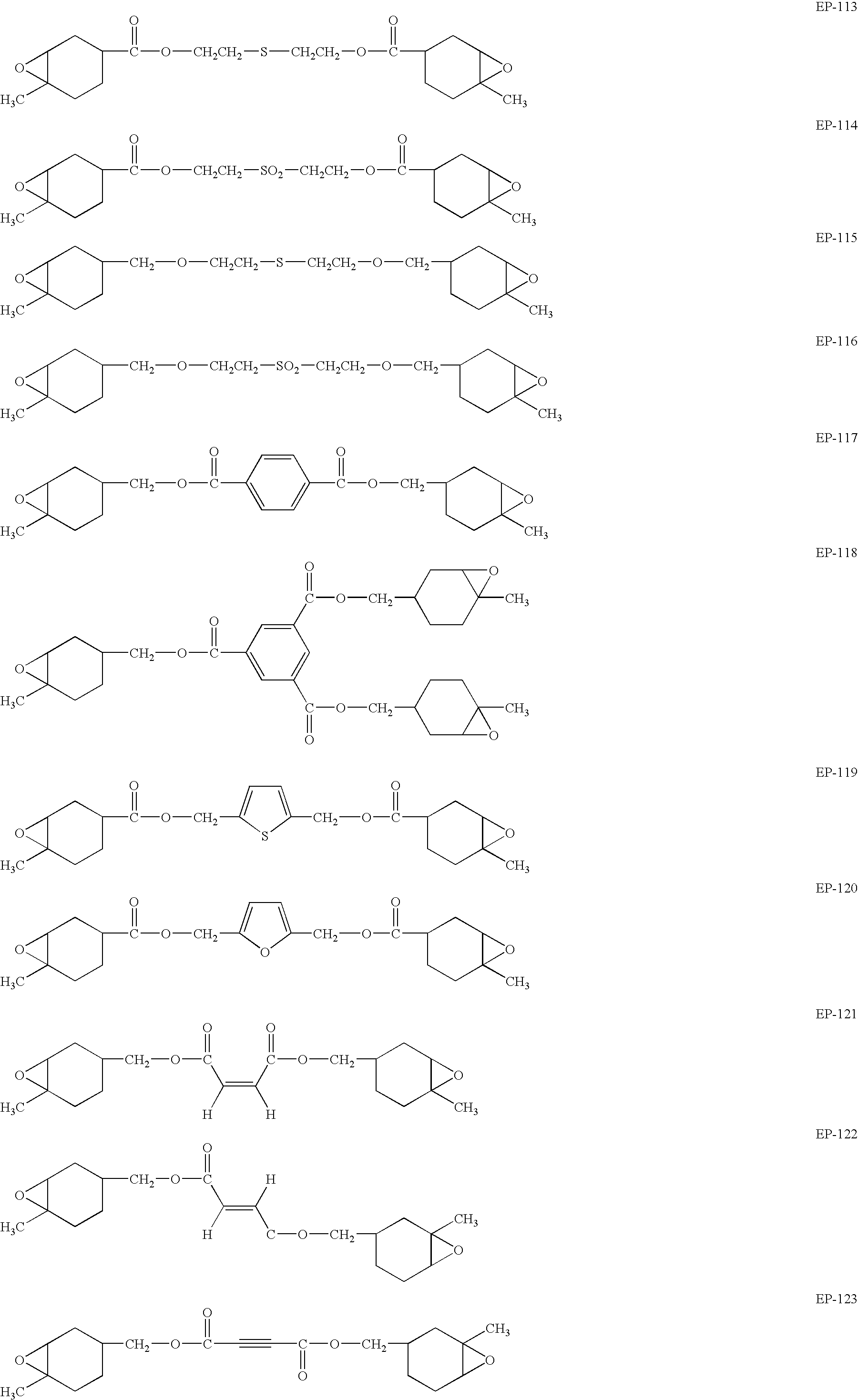

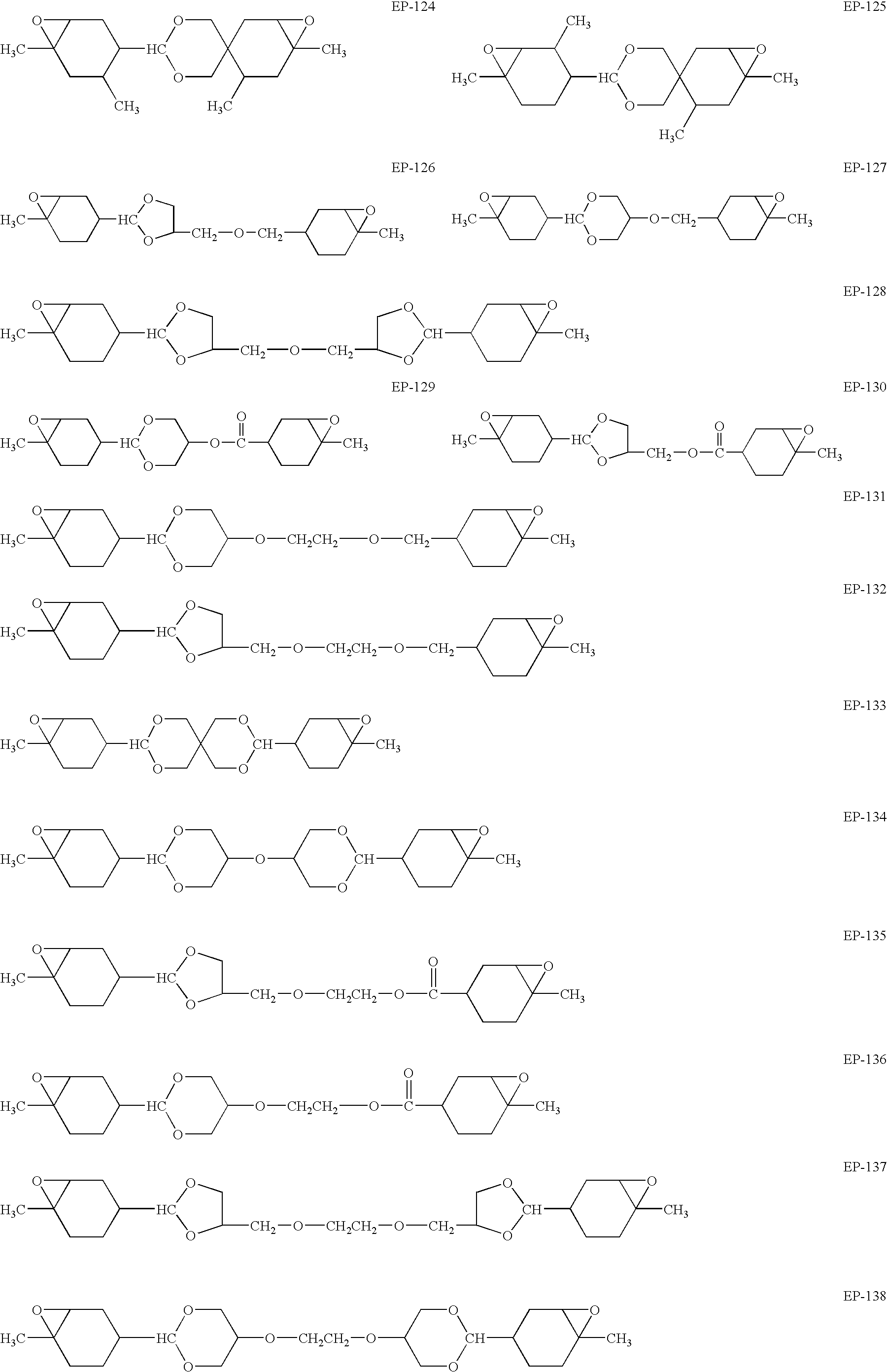

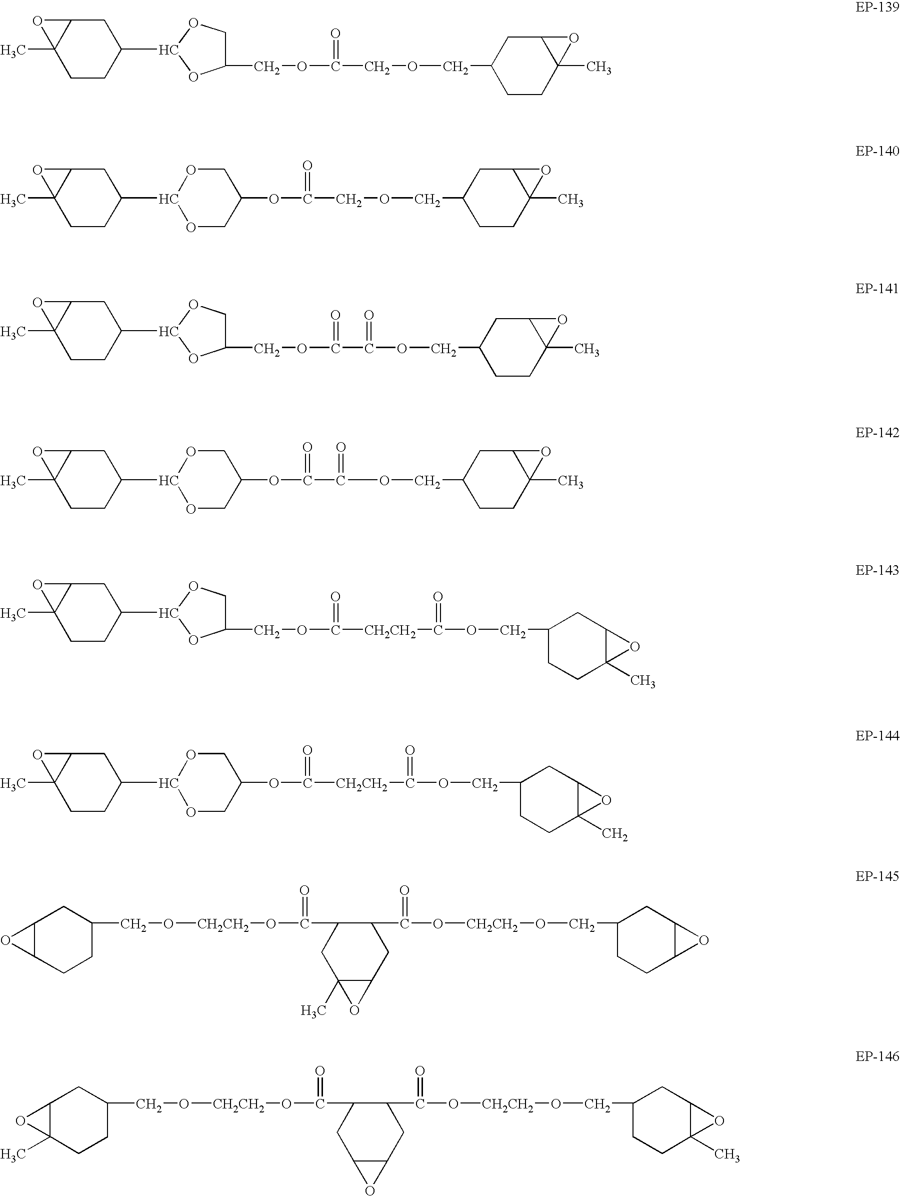

The added amount of the epoxy compound other than those represented by Formula (X) is preferably 10-40 weight %, from the viewpoint of curing capability under curing conditions (being temperature and humidity) and physical properties of the film after curing. The more preferable added amount is 20-40 weight %. In this invention, one type of a multi-functional epoxy compound may be utilized alone or at least two types may be utilized in appropriate combinations.

These alicyclic epoxy compounds, although the manufacturing method is not specifically limited, can be synthesized referring to, for example, “The fourth edition, Experimental Chemistry Course 20, Organic Synthesis II, p. 213-, published by Maruzen Co., Ltd. (1992)”; “The Chemistry of Heterocyclic Compounds—Small Ring Heterocycles part 3, Oxiranes”, edited by Alfred Hasfner, John & Willey and Sons, An Interscience Publication, New York (1985); Yoshimura, Adhesion vol. 29, No. 12, 32 (1985), Yoshimura, Adhesion vol. 30, No. 5, 42 (1986), Yoshimura, Adhesion vol. 30, No. 7, 42 (1986), and JP-A Nos. 11-100378, 4-36263 and 4-69360.

The active ray curable composition of this invention preferably contains a polymerization initiator, a preferable example of which is a compound which generates an acid after irradiation of active rays, and further, as a compound generating an acid via irradiation of active rays, preferred is an onium salt compound. Further, the above onium salt compound is preferably a sulfonium salt compound.

As a compound, generating an acid via irradiation of active rays (being a photo-acid generator) utilized in an active ray curable ink comprised of the active ray curable composition of this invention, a compound utilized in a chemical amplification type photoresist or a photo-cationic polymerization, is employed [please refer to “Organic Materials for Imaging”, pp. 187-192, Bunshin Shuppan (1993)]. Preferable compounds utilized in this invention are listed below.

Firstly, listed are salts of B(C₆F₅)₄⁻, PF₆⁻, AsF₆⁻, SbF₆⁻, and a sulfonate such as p-CH₃C₆H₄SO₃ and p-CF₃SO₃⁻; of aromatic onium compounds such as diazonium, ammonium, iodonium, sulfonium and phosphonium. Counter anions are preferably those having a borate compound and PF₆⁻ salt because of their exceptional acid generation capability. Specific examples of an onium compound will be cited below.

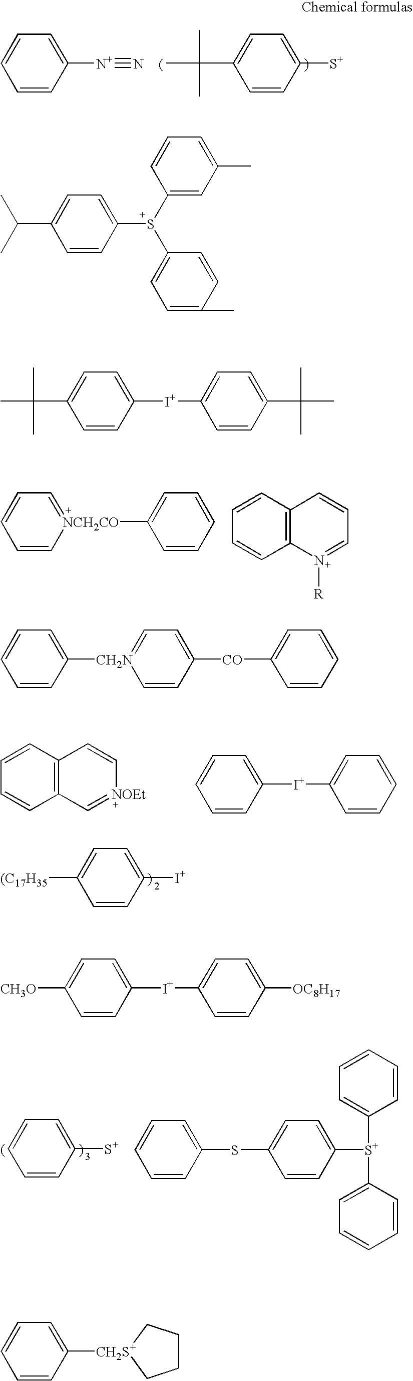

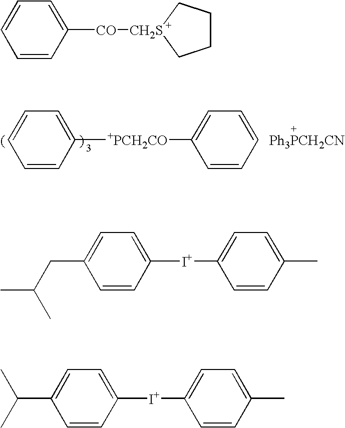

Secondly, listed are sulfonated compounds which generate sulfonic acid. Specific compounds of which will be exemplified below.

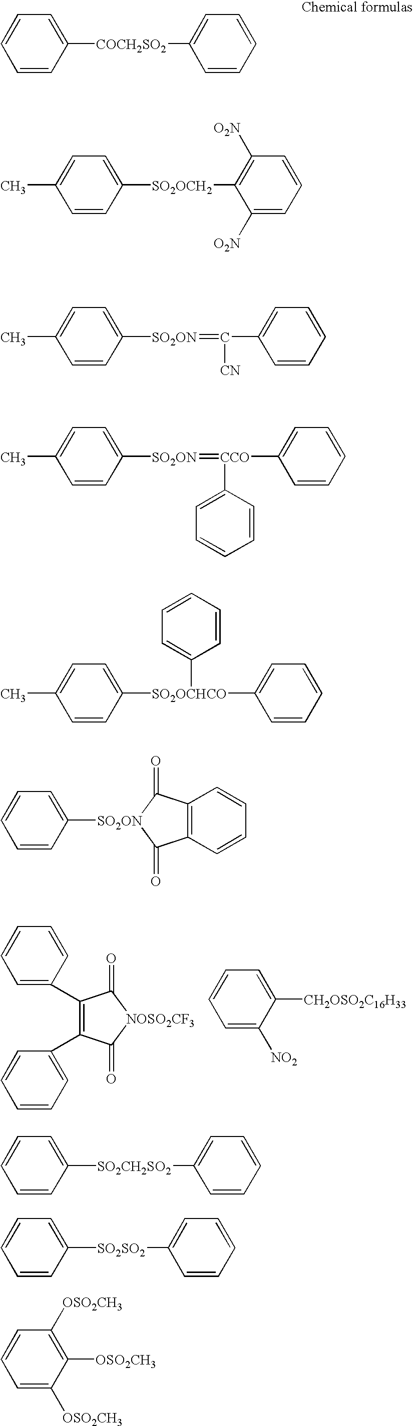

Thirdly, halogenides which generate hydrogen halogenide can also be utilized. Specific compounds of these will be exemplified below.

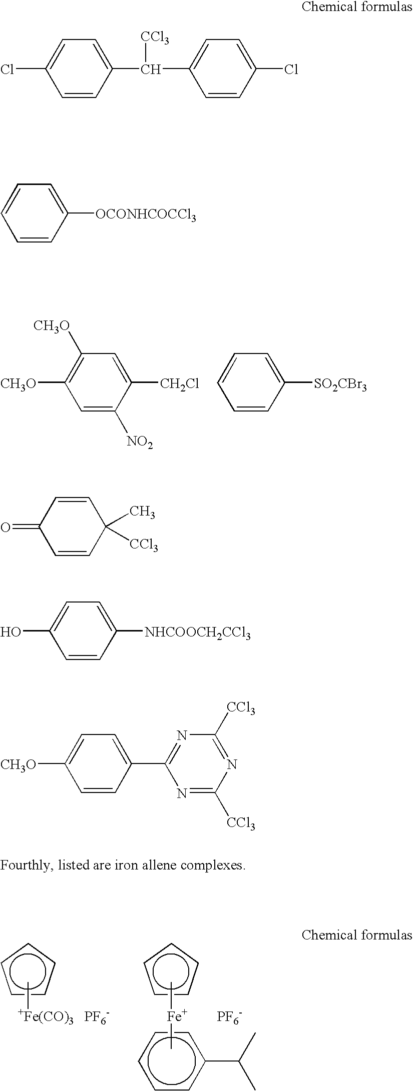

A cationic photo-polymerization initiator utilized in this invention includes a photo-acid generator such as aryl sulfonium salt derivatives (such as Cylacure UVI-6990 and Cylacure UVI-6974 produced by Union Carbide Corp.; Adekaoptomer SP-150, Adekaoptomer SP-152, Adekaoptomer SP-170 and Adekaoptomer SP-172 produced by Asahi Denka Co., Ltd.); allyl iodonium salt derivatives (such as RP-2074, produced by Rohdia Corp.); allene-ion complex derivatives (such as Irgacure 261, produced by Ciba Geigy Corp.); and diazonium salt derivatives; triazine type initiators; and other halogenides. A cationic photo-polymerization initiator is preferably incorporated at a ratio of 0.2-20 weight parts based on 100 weight parts of a compound exhibiting cationic polymerizable capability. It is problematic to prepare a cured product when the content of photo-polymerization initiator is less than 0.2 weight parts, while there is no further enhancing effect of curing when the amount is over 20 weight parts. These photo-cationic polymerization initiators may be utilized by selecting one type or not less than two types.

A photo-acid generator utilized in this invention is preferably onium salt such as sulfonium salt, iodonium salt, ammonium salt and phosphonium salt, and among them preferable is the sulfonium salt compound. A structure of a more preferable sulfonium salt compound includes sulfonium compounds represented by previously cited Formulas (I-1), (I-2) or (I-3).

In Formula (I-1), R₁₁, R₁₂ and R₁₃ are each a substituent group. Examples of such a substituent group include a halogen atom (such as a chlorine atom, a bromine atom or a fluorine atom); an alkyl group having a carbon number of 1-6 (such as a methyl group, an ethyl group, a propyl group, an isopropyl group and a butyl group); a cycloalkyl group having a carbon number of 3-6 (such as a cyclopropyl group, a cyclobutyl group, a cyclopentyl group and a cyclohexyl group); an alkenyl group having a carbon number of 1-6 (such as a vinyl group, a 1-propenyl group, a 2-propenyl group and a 2-butenyl group); an alkynyl group having a carbon number of 1-6 (such as an acetylenyl group, a 1-propinyl group, a 2-propinyl group and a 2-butynyl group); an alkoxy group having a carbon number of 1-6 (such as a methoxy group, an ethoxy group, an n-propoxy group, an iso-propoxy group, an n-butoxy group and a tert-butoxy group); an alkylthio group having a carbon number of 1-6 (such as a methylthio group, an ethylthio group, an n-propylthio group, an iso-propylthio group, an n-butylthio group and a tert-butylthio group); an aryl group having a carbon number of 6-14 (such as a phenyl group, a naphthyl group and an anthracenyl group); an aryloxy group having a carbon number of 6-10 (such as an phenoxy group and a naphthoxy group); an arylthio group having a carbon number of 6-10 (such as a phenylthio group and a naphthylthio group); an acyl group (such as an acetyl group, a propionyl group, a trifluoroacetyl group and a benzoyl group); an acyloxy group (such as an acetoxy group, a propionyloxy group, a trifluoroacetoxy group and a benzoyloxy group); an alkoxycarbonyl group (such as a methoxycarbonyl group, an ethoxycarbonyl group and a tert-butoxycarbonyl group); a hetero atom containing aromatic ring group having a carbon number 4-8 (such as a furyl group and a thienyl group); a nitro group; and a cyano group. Preferable substituents are a halogen atom, an alkyl group, an alkoxyl group, an aryl group, an aryloxy group, an arylthio group and an acyl group. Among these substituent groups, those which can be substituted may if possible be further substituted.

“m”, “n”, and “p” are each an integer of 0-2, and are each preferably at least 1.

X₁₁⁻ is a counter ion, which includes a complex ion such as BF₄⁻, B(C₆F₆)₄⁻, PF₆⁻, AsF₆⁻ and SbF₆⁻; and a sulfonate ion such as p-CH₃C₆H₄SO₃⁻ and CF₃SO₃⁻. As a counter ion, a borate ion and PF₆⁻ are preferable due to their high acid generation capability.

In Formula (I-2), R₁₄ is identical to R₁₁, R₁₂ and R₁₃. “q” is an integer of 0-2, preferably at least 1, but more preferably 2.

Further, R₁₅ and R₁₆ are a substituted or an unsubstituted alkyl group, a substituted or unsubstituted alkenyl group, a substituted or an unsubstituted alkynyl group, or a substituted or an unsubstituted aryl group. Examples of a substituent group are a halogen atom (such as a chlorine atom, a bromine atom and a fluorine atom); an alkyl group having a carbon number of 1-6 (such as a methyl group, an ethyl group, a propyl group, an isopropyl group and a butyl group); a cycloalkyl group having a carbon number of 3-6 (such as a cyclopropyl group, a cyclobutyl group, a cyclopentyl group and a cyclohexyl group); an alkenyl group having a carbon number of 1-6 (such as a vinyl group, a 1-propenyl group, a 2-propenyl group and a 2-butenyl group); an alkynyl group having a carbon number of 1-6 (such as an acetylenyl group, a 1-propinyl group, a 2-propinyl group and a 2-butynyl group); an alkoxy group having a carbon number of 1-6 (such as a methoxy group, an ethoxy group, an n-propoxy group, an iso-propoxy group, an n-butoxy group and a tert-butoxy group); an alkylthio group having a carbon number of 1-6 (such as a methylthio group, an ethylthio group, an n-propylthio group, an iso-propylthio group, an n-butylthio group and a tert-butylthio group); an aryl group having a carbon number of 6-14 (such as a phenyl group, a naphthyl group and an anthracenyl group); an aryloxy group having a carbon number of 6-10 (such as an phenoxy group and a naphthoxy group); an arylthio group having a carbon number of 6-10 (such as a phenylthio group and a naphthylthio group); an acyl group (such as an acetyl group, a propionyl group, a trifluoroacetyl group and a benzoyl group); an acyloxy group (such as an acetoxy group, a propionyloxy group, a trifluoroacetoxy group and a benzoyloxy group); an alkoxycarbonyl group (such as a methoxycarbonyl group, an ethoxycarbonyl group and a tert-butoxycarbonyl group); a hetero atom containing aromatic ring group having a carbon number 4-8 (such as a furyl group and a thienyl group); a nitro group; and a cyano group. Preferable substituent groups are the halogen atom, the alkyl group, the alkoxy group, the aryloxy group and the acyl group.

X₁₂⁻ is identical to X₁₁⁻.

In Formula (I-3), R₁₇ is identical to R₁₁, R₁₂ and R₁₃. “r” is an integer of 0-3, preferably at least 1, but more preferably 2. R₁₈ is a hydrogen atom, or a substituted or unsubstituted alkyl group, while R₁₉ and R₂₀ are identical to R₁₅ and R₁₆.

X₁₃⁻ is identical to X₁₁⁻.

In the following, specific examples of a sulfonium salt compound represented by Formula (I-1), (I-2) or (I-3) will be shown, however, this invention is not limited thereto.

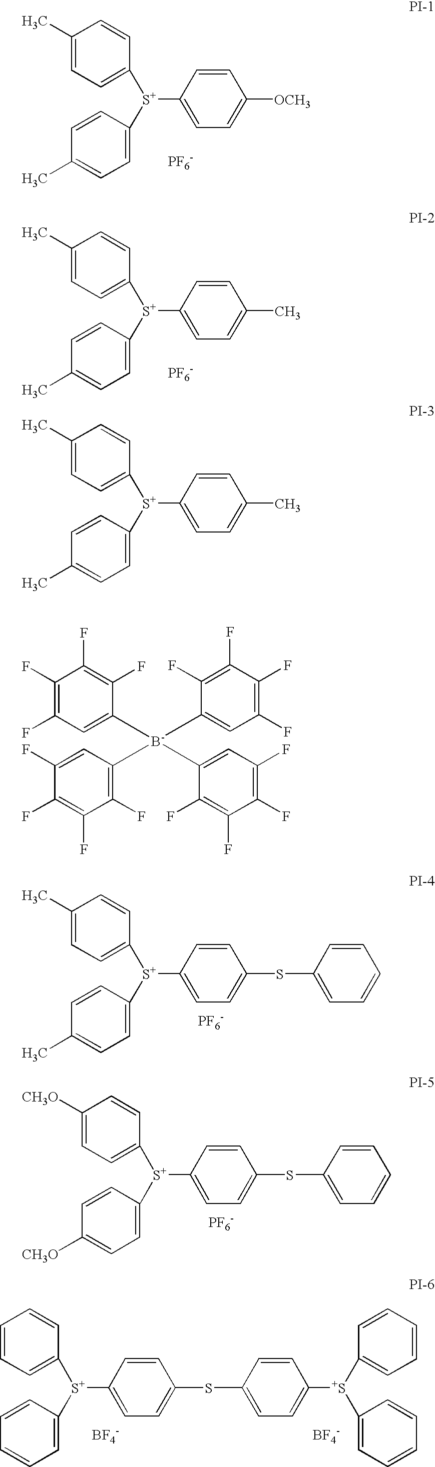

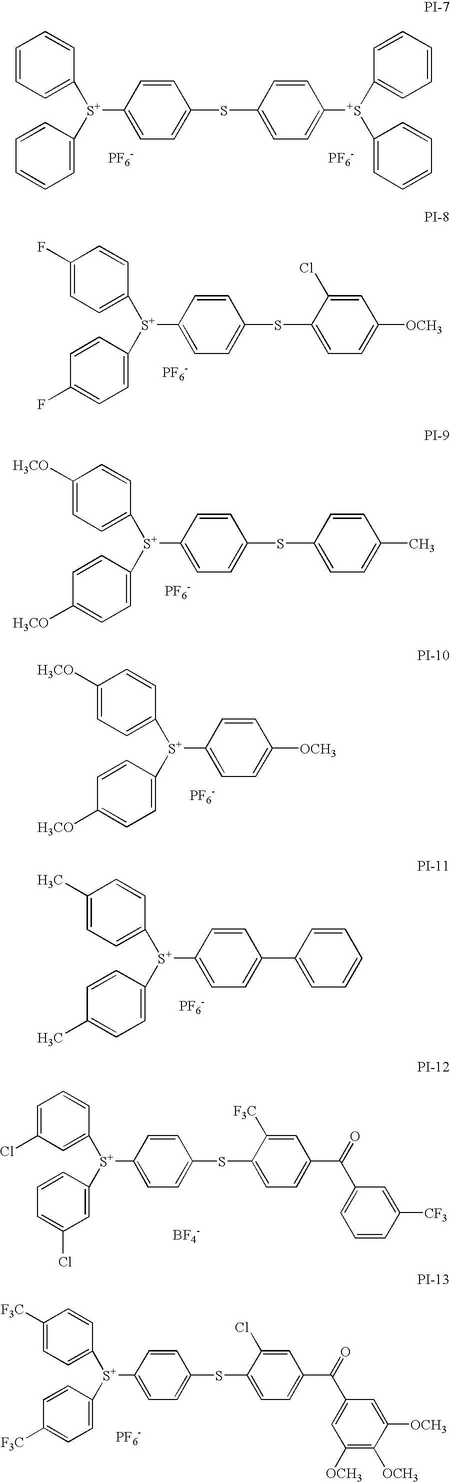

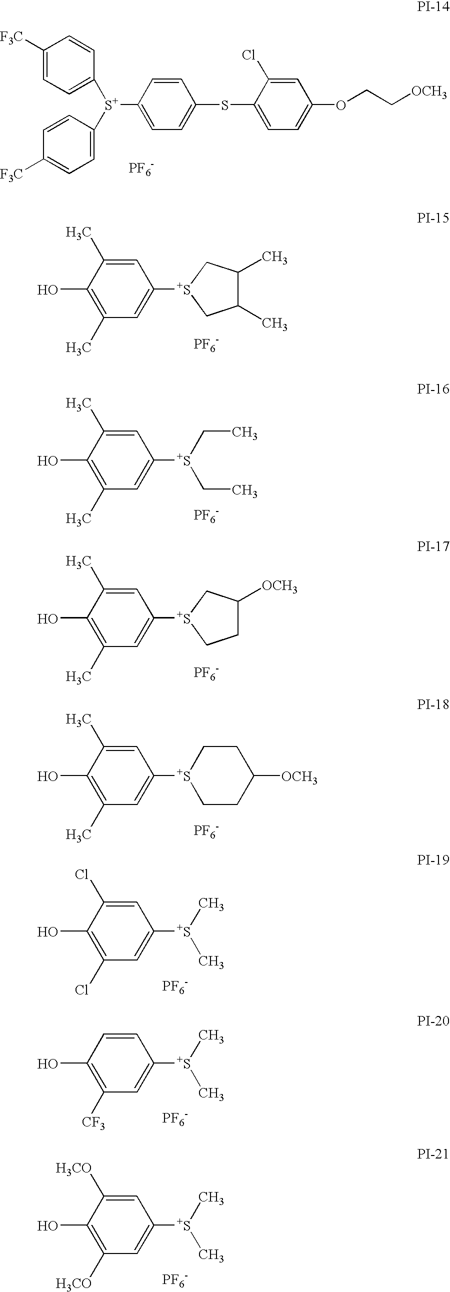

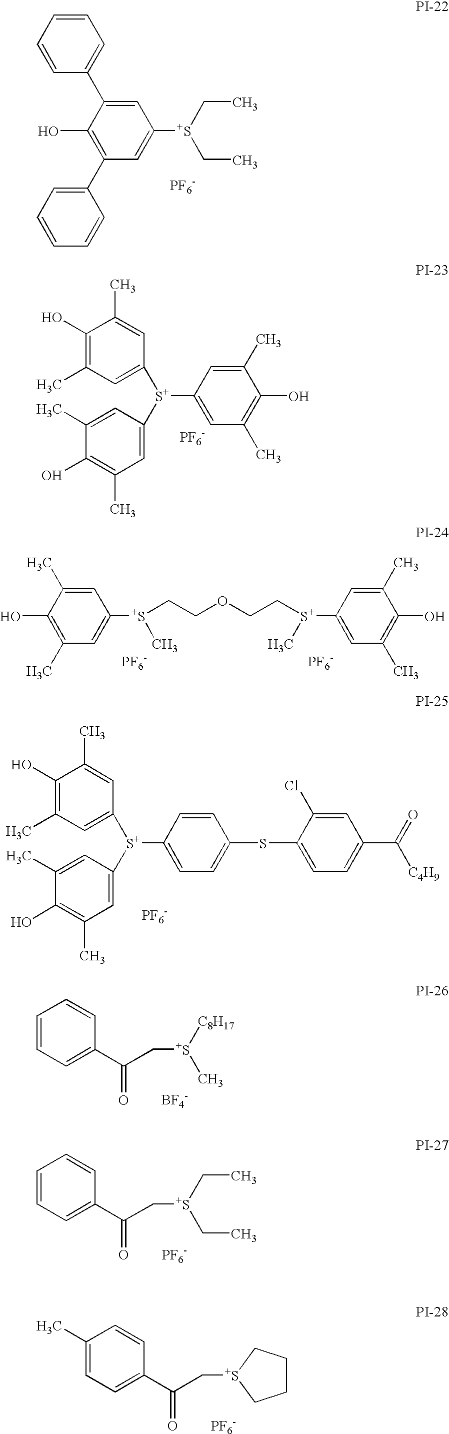

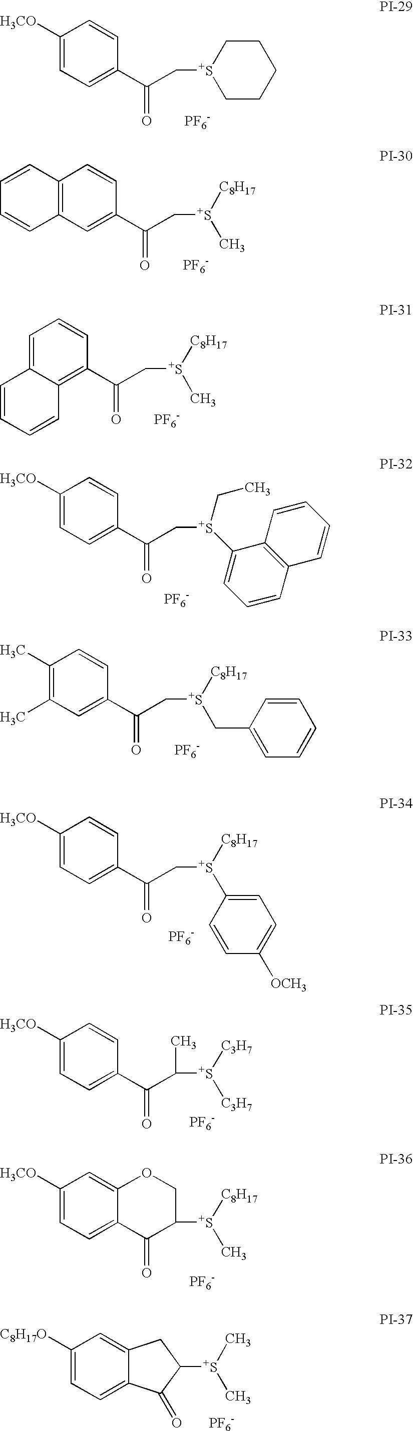

A photo-polymerization accelerator includes anthracene, anthracene derivatives (such as Adekaoptomer SP-100, produced by Asahi Denka Co., Ltd.), phenothiazine (10H-phenothiazine) and phenothiazine derivatives (such as 10-methylphenothiazine, 10-ethylphenothiazine, 10-decylphenothiazine, 10-acetylphenothiazine, 10-decylphenothiazine-5-oxide, 10-decylphenothiazine-5,5-dioxide and 10-acetylphenothiazine-5,5-dioxide). One type, or a combination of plural types, of these photo-polymerization accelerators, may be utilized.

In the active ray curable composition of this invention, various types of additives, other than the constituent elements described above, may be utilized.

As a color material utilized in the active ray curable ink comprised of the active ray curable composition of this invention and in ink-jet ink (hereinafter, also described as “ink-jet ink of this invention”), utilized may be a color material capable of being dissolved or dispersed in the primary component of a photo polymerizable compound, however, a pigment is preferable with respect to weather resistance.

Preferable pigments utilized in this invention are listed below.

C. I. Pigment Yellow-1, 3, 12, 13, 14, 17, 42, 81, 83, 87, 95, and 109,

C. I. Pigment Orange-16, 36, and 38,

C. I. Red-5, 22, 38, 48:1, 48:2, 48:4, 49:1, 53:1, 57:1, 63:1, 101, 144, 146, and 185,

C. I. Pigment Violet-19, and 23,

C. I. Pigment Blue-15:1, 15:3, 15:4, 18, 27, 29, and 60,

C. I. Pigment Green-7, and 36,

C. I. Pigment White-6, 18, and 21,

C. I. Pigment Black-7,

For the purpose of dispersion of the above pigments, devices such as a ball mill, a sand mill, an atliter, a roll mill, an agitator, a Henschel mixer, a colloidal mill, an ultrasonic homogenizer, a pearl mill, a wet-jet mill and a paint shaker may be utilized. Further, it is also possible to incorporate a dispersing agent at the time of dispersion of the pigments. As a dispersing agent, preferably utilized is a polymer dispersing agent which includes the Solsperse series produced by Avecia Corp. Further, a synergist corresponding to various types of pigment as a dispersion aid may also be utilized. These dispersing agents and dispersion aids are preferably added at 1-50 weight parts to 100 weight parts of pigment. A solvent or a polymerizing compound may be utilized as a dispersion medium, however, in an active ray curable ink utilized in this invention, a solvent-free type is preferred since reaction and curing are performed immediately after ink deposition. When any solvent remains in a cured image, caused may be problems such as of deterioration of solvent resistance and of VOC (Volatile Organic Compounds) problem of a residual solvent. Therefore, a dispersion medium is preferably not a solvent but a photo polymerizable compound, and monomers exhibiting the lowest viscosity among them is more preferably selected with respect to dispersion adaptability.

For dispersion of the pigments, it is preferable to make the pigment particles size 0.08-0.5 μm, and selection of pigment, a dispersing agent and a dispersion medium, dispersion condition, and filtration condition are appropriately set so as to make the maximum particle size be 0.3-10 μm, but preferably be 0.3-3 μm. Via such particle size control, clogging of head nozzles is reduced, and storage stability, transparency and curing sensitivity of ink can be ensured.

In the ink-jet ink of this invention, the pigment concentration is preferably 1-10 weigh % based on the total weight of the ink.

In this invention, a heat-base generator can also be utilized to improve ejection stability and storage stability of the ink.

As a heat-base generator, for example, a salt of organic acid which decomposes via decarboxylation under heat, and a base, a compound which release amines by decomposition via a reaction such as intramolecular neucleophilic substitution, Lossen rearrangement and Beckman's rearrangement, and those releasing a base by causing some reaction with heat are preferably utilized. Specifically listed are the salt of trichloroacetic acid described in British Patent No. 998,949, a salt of α-sulfonylacetic acid described in U.S. Pat. No. 4,060,420, aldoxime carbamate derivatives and 2-carboxycarboxyamide derivatives described in JP-A 59-157637, a salt utilizing alkali metal or alkaline earth metal other than an organic base as a base component with a thermal decomposing acid described in JP-A 59-168440, hydroxame carbamates utilizing Lossen rearrangement described in JP-A 59-180537, and aldoxime carbamates which generate nitrile under heat described in JP-A 59-195237. In addition to these, useful are heat-base generators described in British Patent No. 998,945, U.S. Pat. No. 3,220,846, British Patent No. 279,480, JP-A Nos. 50-22625, 61-32844, 61-51139, 61-52638, 61-51140, 61-53634, 61-53640, 61-55644 and 61-55645.

Further, specific examples include guanidine trichloroacetate guanidine, methylguanidine trichloroacetate, potassium trichloroacetate, guanidine phenylsulfonylacetate, guanidine p-chlorophenylsulfonylacetate, guanidine p-methanesulfonylphenylsulfonylacetate, potassium phenylpropiolate, guanidine phenylpropiolate, cesium phenylpropiolate, guanidine p-chlorophenylpropiolate, guanidine p-phenylene-bisphenylpropiolate, tetramethylammonium phenylsulfonylacetate and tetramethylammonium phenylpropiolate. The above-cited heat-base generators can be utilized in a wide range of applications.

The ink-jet ink of this invention can also contain an acid multiplier, which newly generates an acid by an acid generated by active ray irradiation, well-known in the art according to such as JP-A Nos. 8-248561 and 9-34106.

The ink-jet ink of this invention is produced by sufficiently dispersing pigments together with an active ray curable composition and a pigment dispersing agent by use of a homogenizer such as a sand mill. It is preferable to dilute a concentrated solution of pigment at a high concentration, which has been prepared in advance, with an active ray-curable compound. Sufficient dispersion is possible by dispersion employing an ordinary homogenizer, and therefore, since no excess dispersion energy is applied nor long dispersion time is required, alternation of ink component at the time of dispersion rarely results and ink of superior stability is produced. The ink is preferably filtered through a filter having a pore diameter of at most 3 μM, but more preferably of at most 1 μm.

The active ray-curable composition of this invention is preferably adjusted to have a viscosity of 1-500 mPa·s at 25° C., from the viewpoint of following capability of ink ejection from a high frequency ink-jet head and at excellent ink ejection stability. The active ray-curable ink of this invention is preferably adjusted to exhibit a viscosity of 7-40 mPa·s at 25° C., which is assumed to be slightly high level. Ink of a viscosity of 7-40 mPa·s at 25° C. specifically exhibits stable ejection characteristics with an ink-jet head of a normal frequency of 4-10 KHz through a high frequency of 10-50 KHz.

Further, the ink-jet ink of this invention is preferably prepared as ink which exhibits a conductivity of at most 10 μS/cm, when used with a piezo head, and is preferred as an ink which does not result in electrical corrosion in the interior of the head. Further, with a continuous type head, electrical conductivity is typically adjusted via an electrolyte, and in this case, the conductivity is required to be adjusted to at least 0.5 mS/cm.

In this invention, surface tension of ink at 25° C. is preferably in the range of 25-40 mN/m from the viewpoint of ejection stability and deposited ink dot diameter stability.

An appropriate surface active agent may be incorporated to adjust the surface tension. A surface active agent preferably utilized in the ink-jet ink of this invention includes an anionic surface active agent such as dialkylsulfosuccinates, alkylnaphthalenesulfonates and fatty acid salts; a nonionic surface active agent such as polyoxyethylene alkyl ethers, polyoxyethylene alkylallyl ethers, acetylene glycols and polyoxyethylene.polyoxypropylene block copolymers; a cationic surface active agent such as alkylamines and quaternary ammonium salts; and a surface active compound having a polymerizing group. Of these, specifically preferred is a surface active compound having a polymerizable group such as an unsaturated bond and an oxirane or an oxetane ring, for example, silicone modified acrylate, fluorine modified acrylate, silicone modified epoxy, fluorine modified epoxy, silicone modified oxetane, and fluorine modified oxetane.

In the ink-jet ink of this invention, various additives other than those noted above may be utilized. For example, added may be a leveling agent, a matting agent, polyester type resin, polyurethane type resin, vinyl type resin, acrylic type resin, rubber type resin and waxes to optimally adjust physical film properties. Addition of a slight amount of an organic solvent is also effective to enhance adhesion to a recording medium. In this case, addition in a range to not cause problems of solvent resistance and VOC is essential, and the added amount is typically in the range of 0.1-5%, but preferably 0.1-3%. Further, by combining with a radical polymerizable monomer and an initiator, it is also possible to make a radical-cation hybrid type curable ink.

In an image forming method of this invention, an ink composition is ejected to create an image on a recording material via an ink-jet recording method and successively the ink is cured by irradiation of active rays such as ultraviolet rays.

In an image forming method of this invention, it is preferable to heat ink, as well as the ink-jet nozzles, to liquefy the ink and provide it at a low viscosity during ink ejection. The heating temperature is typically 30-100° C., but preferably 35-100° C.

In this invention, the total ink layer thickness after ink has been deposited and cured by irradiation of active rays is preferably 2-20 μm. Specifically in the soft package printing field, a thickness of 2-20 μm is preferable from the viewpoint of preventing curling and wrinkling of the recording medium and also stiffness and texture of the printed medium.

Further, in this invention, the droplet volume ejected from each nozzle is preferably 2-15 pl.

In this invention, to form a high precision image, it is preferable that irradiation timing is as fast as possible, however, it is preferable, in this invention, to start light irradiation only when viscosity or water content of ink reaches a preferable state.

Specifically, as an irradiation condition of active rays, it is preferable to start active ray irradiation within 0.001-2.0 seconds after ink deposition, but more preferably within 0.001-1.0 second. Further, it is preferable to finish light irradiation when the ink loses fluidity, typically in 0.1-3 seconds but preferably 0.2-1 second. By creating to the above conditions, it is possible to prevent enlargement of a dot diameter and bleeding among dots.

As an irradiation method of active rays, a basic method is disclosed in JP-A 60-132767. According to this, light sources are provided on both sides of a recording head, and the recording head and the light sources are reciprocally scanned via a shuttle method. Irradiation is conducted a specific time after ink deposition. Further, curing is completed by a separate undriven light source. In U.S. Pat. No. 6,145,979, disclosed are, a method utilizing an optical fiber, and a method in which a collimated light source is incident to a mirror arranged on the side of the recording head unit and UV light is irradiated on the recorded portion. In an image forming method of this invention, any one of these irradiation methods may be utilized.

Further, a preferable embodiment is a method in which irradiation of active rays is divided into two stages, to firstly irradiate active rays via the aforesaid method within 0.001-2.0 seconds after ink deposition, after which active rays are further irradiated after finished printing of the whole page. By dividing irradiation of active rays into two stages, it is possible to reduce shrinkage of the recording medium which is typically caused during ink curing.

Examples of light sources utilized for active ray irradiation include a mercury arc lamp, a xenon arc lamp, a fluorescent arc lamp, a carbon arc lamp, a tungsten-halogen copying lamp, a high pressure mercury lamp, a metal halide lamp, a non-electrode UV lamp, a low pressure mercury lamp, a UV laser, a xenon flash lamp, an insect electrocution lamp, a black light, a sterilizing lamp, a cold cathode tube and an LED, however, these are not limited thereto, and among them preferable is the fluorescent lamp because of its low energy requirements and low cost. Wavelength of the light source is preferably in the range of 250-370 nm but more preferably of 270-320 nm based on the peak of the emission wavelength with respect to sensitivity. Illuminance is typically 1-3,000 mW/cm² but preferably 1-200 mW/cm². Further, in the case of curing with electron rays, generally electron rays at an energy of at most 300 eV are employed, however, it is also possible to employ an irradiation amount of 1-5 Mrad to achieve almost instantaneous curing.

Image printing on a recording medium (also referred to as a substrate) can be performed utilizing the ink-jet ink of this invention, and as a recording medium, all the wide range of synthetic resins, which are conventionally utilized in various applications, may be employed, including such as polyester, polyvinyl chloride, polyethylene, polyurethane, polypropylene, acrylic resin, polycarbonate, polystyrene, acrylonitrile-butadiene-styrene copolymer, and polyethyleneterephthalate; the thickness and form of these resin substrates are not limited.

As a substrate utilizable in this invention, a non-absorptive support, in addition to such as ordinary non-coated paper and coated paper, may be employed, however, the non-absorptive supports are preferable.

In this invention, as a non-absorptive support, various types of plastics and films thereof may be utilized, as well as various plastic films including such as PET film, OPS film, OPP film, ONy film, PVC film, PE film and TAC film. In addition to these, such as polycarbonate, acrylic resin, ABS, polyacetal, PVA and appropriate types of rubber may be utilized. Further, metals and various types of glass are also applicable. Among these recording media, one embodiment of this invention is effective in the case of forming an image on PET film, OPS film, OPP film, ONy film and PVC film, all of which are capable of shrinkage via heat. In these substrates, curling and deformation of film easily result by such as curing shrinkage of ink and heating during the curing reaction, and ink film exhibits difficulty in following shrinkage of a substrate.

The surface energy of these various types of plastic films significantly differs and one problem has been heretofore that the dot diameter changes after ink deposition, depending on a recording medium. One embodiment of this invention enables using OPP film and OPS film having a small surface energy to PET film having a relatively large surface energy, however, a substrate preferably exhibits a wet index of 40-60 mN/m.

In this invention, a long roll of (namely a web) recording medium is advantageously utilized, with respect to cost of the recording material such as expense of packaging and manufacturing, preparation efficiency for printing, and adaptability to various sizes of print.

Next, the ink-jet recording apparatus of this invention will be described.

In the following, the ink-jet recording apparatus of this invention will be described referring to appropriate drawings. Herein, the recording apparatus of the drawings is only one embodiment of the ink-jet recording apparatus of this invention, and the ink-jet recording apparatus of this invention is not limited to the drawings.

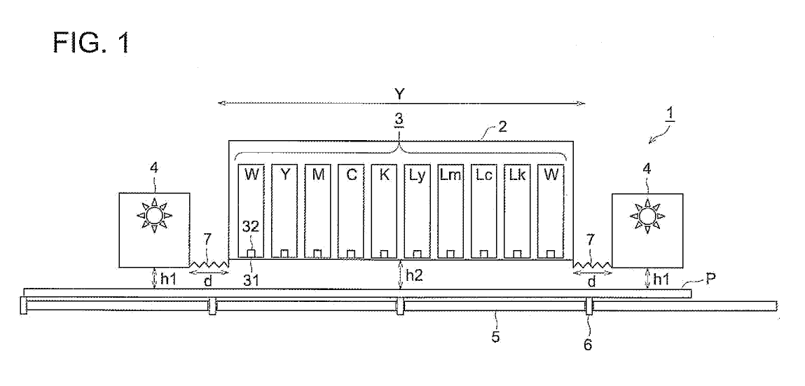

FIG. 1 shows a front view of an embodiment of the primary portion of the recording apparatus of this invention. Recording apparatus 1 is constituted of such system as head carriage 2, recording head 3, irradiation means 4, and platen portion 5. In this recording apparatus 1, platen portion 5 is arranged below recording medium P. Platen portion 5 functions to absorb ultraviolet rays and absorbing excess ultraviolet rays having passed through recording medium P. As a result, an image of high resolution can be consistently reproduced.

Recording medium P is guided by guide members 6 to be conveyed to the interior of FIG. 1 by operation of a transfer means (not shown in the drawing). A head scanning means (also not shown in the drawing) reciprocally scans recording head 3 mounted on head carriage 2 via shifting head carriage 2 along direction Y in FIG. 1.

Head carriage 2 is arranged above recording medium P to store a plural number of recording heads 3, which will be described later, corresponding to the number of colors required for targeted image printing on recording medium P while arranging the ejection outlet downward. Head carriage 2 is arranged to freely reciprocate within the main body of recording apparatus 1, and is shuttled back and forth along direction Y of FIG. 1, via a head scanning means drive.

Herein, in FIG. 1, head carriage 2 is drawn so as to store recording heads 3 of yellow (Y), magenta (M), cyan (C) black (K) and white (W), however, in practical use, the number of colors of recording heads 3 to be stored in head carriage 3 can be determined as appropriate.

Recording head 3 ejects active ray-curable ink-jet ink (for example, UV-curable ink), which has been supplied from an ink supply means (not shown in the drawing) onto recording medium P through an ejection outlet via operation of an ejection means (not shown in the drawing), a plural number of which are stored in recording head 3. UV ink, ejected from recording head 3, is comprised of such as a color material, a polymerizing monomer and an initiator, and further is provided with the capability of curing by cross-linking or polymerization of the monomer capable of catalytic reaction of the initiator due to irradiation of ultraviolet rays.

Recording head 3 ejects the ultraviolet ray-curable ink as ink drops on a predetermined region of recording medium (the region designated for ink deposition) to deposit ink drops on the targeted region during a scan of being shifted from one edge of recording medium P to the other edge of recording medium P along direction Y in FIG. 1 by a drive of the head scanning means.

After the above-described scan is performed an appropriate number of times to eject the ultraviolet ray-curable ink at a region designated for ink deposition, recording medium P is shifted farther to the interior of FIG. 1 by a shifting means and the ultraviolet ray curable ink is ejected onto the next region for ink deposition adjacent to the above-designated region while repeating another scan by a shifting the head scanning means again.

By repeating the above-described operations which eject ultraviolet ray-curable ink from recording head 3 synchronously with the head scanning means and the recording medium P transfer means, an image comprising an aggregate of ultraviolet curable ink drops is formed on recording medium P.

Irradiation means 4 is constituted of an ultraviolet lamp, which emits ultraviolet rays of a specific wavelength range at a stable exposure energy, and a filter, which allows passage of ultraviolet rays of only a specific wavelength region. Herein, as an ultraviolet lamp, such as a mercury lamp, a metal-halide lamp, an excimer laser, an ultraviolet laser, a cold-cathode tube, a black-light and an LED (being a Light Emitting Diode) are applicable, of which a metal-halide lamp, a cold-cathode tube, a mercury lamp or a black-light, all of which are belt-like, are preferable. Specifically, a cold-cathode tube and a black-light, which emit ultraviolet rays of a wavelength of 365 nm, are most preferred because prevention of bleeding and control of a dot diameter can be efficiently achieved as well as decreasing wrinkles during curing. By employing a black-light as a radiation source for irradiation means 4, irradiation means 4 to cure ultraviolet ray-curable ink can be prepared at low cost.

Irradiation means 4 is structured of a form approximately same as the maximum range which can be accommodated by recording apparatus 1, or larger than the region designated to carry an image, among the regions to receive ultraviolet ray-curable ink ejected by recording head 3 during one scan by drive of the head scanning means.

Irradiation means 4 is arranged to be fixed nearly parallel to recording medium P on both sides of head carriage 2.

As described before, as a means to adjust illuminance onto ink ejection portions, the entire recording head 3 is of course light-shielded, and in addition, it is effective to set distance h2 between ink ejection portion 31 of recording head 3 and recording medium P to be larger than distance h1 between irradiation means 4 and recording medium P (h1<h2), or to increase distance d between recording head 3 and irradiation means 4 (for example, to make d larger). Further, it is more preferable to provide bellows structure 7 between recording head 3 and irradiation means 4.

Herein, the wavelength of ultraviolet rays emitted by irradiation means 4 can be optimized by changing the ultraviolet lamp or filter which is housed in irradiation means 4.

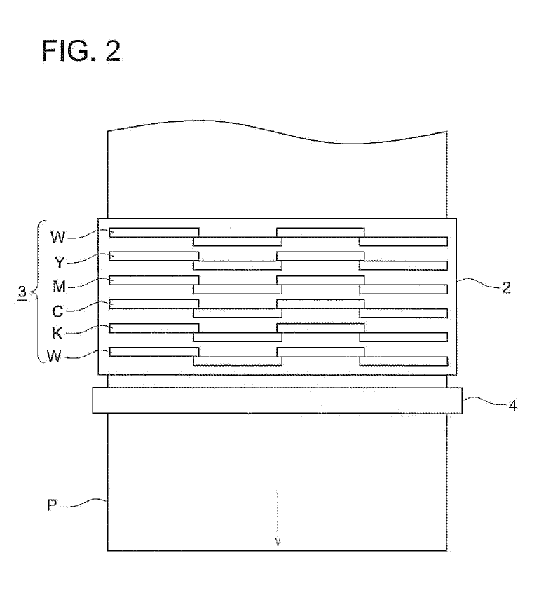

FIG. 2 shows a drawing of another example of a constitution of the primary portion of an ink-jet recording apparatus.

The ink-jet recording apparatus shown in FIG. 2 is called a line head type, in which a plural number of ink-jet recording heads 3 of specific colors are arranged so as to cover the whole width of recording medium P. Head carriage 2 is light shielded.

In this line head type, head carriage 2 and irradiation means 4 are fixed, and only recording medium P is conveyed to form an image by ink ejection and curing.

### EXAMPLES

### Example 1

### Preparation of Active Ray Curable Composition

Active ray curable compositions were prepared via addition and dissolution of a polymerizable compound (10 g), a polymerization initiator (1 g) and a solvent (20 ml) as shown in Tables 1 and 2.

### Polymerization of Active Ray Curable Composition

Polymerization was performed via the following method. The targeted active ray curable composition was stirred while heated to reflux over 30 minutes under a nitrogen atmosphere. Reaction was terminated by addition of 2 g of triethylamine, after which 20 ml of methanol was added, and the resulting precipitate was filtered. The precipitate was thoroughly washed with dichloroethane, and dried under reduced pressure, after which the weight of the precipitate was recorded.

### Evaluation of Active Ray Curable Compositions

Yields of the obtained polymers were compared.

A: The yield was at least 55%.

B: The yield was 40% and more.

C: The yield was between 25-40%.

D: The yield was at most 25%.

Results are shown in Table 1.

The utilized compounds are shown below.
Celloxide 2021P: produced by Daicel Chemical Industries Ltd.
Celloxide 3000: produced by Daicel Chemical Industries Ltd.
Vf70710: Vikoflex 7010 (produced by Atofina S. A.)
OXT-221: produced by Toagosei Co., Ltd.
OXT-212: produced by Toagosei Co., Ltd.

TABLE 1

| | Polymerizable | | | | |
|---|---|---|---|---|---|
| Sample No. | compound | Plymerization initiator | Solvent | Yield | Remarks |
| 1 | X-2 | Trifluoromethanesulfonic | *1 | B | Inv. |
| | | acid | | | |
| 2 | X-3 | Trifluoromethanesulfonic | *1 | A | Inv. |
| | | acid | | | |
| 3 | X-3 | IBPF (produced by Sanwa | *1 | B | Inv. |
| | | Chemical Co., Ltd.) | | | |
| 4 | X-37 | Trifluoromethanesulfonic | *1 | B | Inv. |
| | | acid | | | |
| 5 | X-47 | Trifluoromethanesulfonic | *1 | B | Inv. |
| | | acid | | | |
| 6 | X-68 | Trifluoromethanesulfonic | *1 | A | Inv. |
| | | acid | | | |
| 7 | X-69 | Trifluoromethanesulfonic | *1 | A | Inv. |
| | | acid | | | |
| 8 | X-69 | Trifluoroacetic acid | *1 | B | Inv. |
| 9 | X-72 | Trifluoromethanesulfonic | *1 | A | Inv. |
| | | acid | | | |
| 10 | X-96 | Trifluoromethanesulfonic | *1 | A | Inv. |
| | | acid | | | |
| 11 | X-96 | Trifluoroacecic acid | *1 | B | Inv. |
| 12 | X-2/OXT212 | Trifluoromethanesulfonic | *1 | A | Inv. |
| | (5 g each) | acid | | | |
| 13 | X-3/OXT221 | IBPF (produced by Sanwa | *1 | A | Inv. |
| | (5 g each) | Chemical Co., Ltd.) | | | |
| 14 | Celloxide | Trifluoromethanesulfonic | *1 | C | Comp. |
| | 3000 | acid | | | |
| 15 | Celloxide | Trifluoromethanesulfonic | *1 | C | Comp. |
| | 2021P | acid | | | |
| 16 | Vf7010 | Trifluoromethanesulfonic | *1 | D | Comp. |
| | | acid | | | |

*1: Dichloroethane
Inv.: this invention,
Comp.: comparative exampl

From Table 1, it was proven that polymerization of the active ray curable compositions of this invention advanced better in a relatively short time of polymerization with higher yield compared to the comparative compounds.

### Example 2

### Preparation of Active Ray Curable Compositions

A polymerizable compound, a polymerization initiator, a compound of this invention, and other additives were added and dissolved as shown in Tables 2 and 3 to prepare an active ray curable composition.

TABLE 2

| | Compound | | | | Another | |
|---|---|---|---|---|---|---|
| | of | | Oxetane | | polymerizable | |
| Sample | Formula (X) | | compound | | compound | |
| No. | Kind | *1 | Kind | *1 | Kind | *1 |
| 21 | X-1 | 35 | OXT-212 | 65 | | |
| 22 | X-3 | 35 | OXT-212 | 65 | | |
| 23 | X-4 | 35 | OXT-212 | 65 | | |
| 24 | X-5 | 35 | OXT-212 | 65 | | |
| 25 | X-27 | 35 | OXT-212 | 65 | | |
| 26 | X-37 | 35 | OXT-212 | 65 | | |
| 27 | X-68 | 35 | OXT-212 | 65 | | |
| 28 | X-69 | 35 | OXT-212 | 65 | | |
| 29 | X-71 | 35 | OXT-212 | 65 | | |
| 30 | X-96 | 35 | OXT-212 | 65 | | |
| 31 | X-1 | 30 | OXT-221 | 50 | * | 20 |
| 32 | X-3 | 50 | OXT-221 | 30 | OXT-212 | 20 |
| 33 | X-4 | 40 | OXT-221 | 50 | OXT-212 | 10 |
| 34 | X-5 | 25 | OXT-221 | 65 | *3 | 10 |
| 35 | X-27 | 30 | OXT-221 | 60 | *3 | 10 |
| 36 | X-37 | 50 | OXT-221 | 40 | SEP-56 | 10 |
| 37 | X-68 | 50 | OXT-221 | 40 | SEP-72 | 10 |
| 38 | X-69 | 50 | OXT-221 | 40 | SEP-98 | 10 |
| 39 | X-71 | 50 | OXT-221 | 40 | SEP-102 | 10 |
| 40 | X-96 | 50 | OXT-221 | 40 | SEP-150 | 10 |
| 41 | X-3 | 55 | OXT-212 | 30 | EP-89 | 15 |
| 42 | X-3 | 40 | OXT-221 | 30 | SEP-56 | 30 |
| 43 | X-3 | 40 | OXT-221 | 30 | SEP-72 | 30 |
| 44 | X-69 | 40 | OXT-221 | 30 | SEP-98 | 30 |
| 45 | X-69 | 40 | OXT-221 | 30 | SEP-102 | 30 |
| 46 | X-69 | 40 | OXT-221 | 30 | SEP-150 | 30 |
| 47 | X-71 | 40 | OXT-221 | 30 | SEP-56 | 30 |
| 48 | X-71 | 40 | OXT-221 | 30 | SEP-150 | 30 |
| 49 | X-96 | 40 | OXT-221 | 30 | SEP-56 | 30 |
| 50 | X-96 | 60 | OXT-221 | 30 | EP-89 | 10 |
| 51 | | | OXT-221 | 65 | Celloxide 2021P | 35 |
| 52 | | | OXT-221 | 75 | Celloxide 2021P | 25 |
| 53 | | | OXT-221 | 65 | Vf7010 | 35 |
| 54 | | | OXT-221 | 75 | Vf7010 | 25 |
| 55 | | | OXT-221 | 65 | Celloxide 3000 | 35 |
| 56 | | | OXT-221 | 75 | Celloxide 3000 | 25 |
| 57 | | | OXT-221 | 10 | Epolead GT301 | 90 |

*1: Added amount (weight part)

TABLE 3

| | Photo-acid generator | | Basic compound | | |
|---|---|---|---|---|---|
| Sample | | Added amount | | Added amount | |
| No. | Kind | (weight part) | Kind | (weight part) | Remarks |
| 21 | UVI-6992 | 5.0 | *6 | 0.5 | Inv. |
| 22 | PI-1 | 5.0 | *6 | 0.5 | Inv. |
| 23 | PI-1 | 5.0 | *6 | 0.5 | Inv. |
| 24 | PI-1 | 5.0 | *6 | 0.5 | Inv. |
| 25 | PI-1 | 5.0 | *6 | 0.5 | Inv. |
| 26 | PI-1 | 5.0 | *6 | 0.5 | Inv. |
| 27 | PI-2 | 5.0 | *7 | 0.5 | Inv. |
| 28 | SP-152 | 5.0 | *7 | 0.5 | Inv. |
| 29 | PI-1 | 5.0 | *6 | 0.5 | Inv. |
| 30 | PI-1 | 5.0 | *6 | 0.5 | Inv. |
| 31 | UVI-6992 | 5.0 | *6 | 0.5 | Inv. |
| 32 | PI-1 | 5.0 | *6 | 0.5 | Inv. |
| 33 | PI-2 | 5.0 | *6 | 0.5 | Inv. |
| 34 | UVI-6992 | 5.0 | *6 | 0.5 | Inv. |
| 35 | UVI-6992 | 5.0 | *7 | 0.5 | Inv. |
| 36 | PI-1 | 5.0 | *7 | 0.5 | Inv. |
| 37 | PI-1 | 5.0 | *7 | 0.5 | Inv. |
| 38 | PI-1 | 5.0 | *7 | 0.5 | Inv. |
| 39 | PI-1 | 5.0 | *7 | 0.5 | Inv. |
| 40 | PI-1 | 5.0 | *7 | 0.5 | Inv. |
| 41 | PI-1 | 5.0 | *6 | 0.5 | Inv. |
| 42 | PI-1 | 5.0 | *6 | 0.5 | Inv. |
| 43 | PI-1 | 5.0 | *6 | 0.5 | Inv. |
| 44 | PI-1 | 5.0 | *7 | 0.5 | Inv. |
| 45 | PI-1 | 5.0 | *7 | 0.5 | Inv. |
| 46 | PI-1 | 5.0 | *6 | 0.5 | Inv. |
| 47 | PI-1 | 5.0 | *6 | 0.5 | Inv. |
| 48 | PI-1 | 5.0 | *7 | 0.5 | Inv. |
| 49 | PI-1 | 5.0 | *7 | 0.5 | Inv. |
| 50 | PI-1 | 5.0 | *7 | 0.5 | Inv. |
| 51 | UVI-6992 | 5.0 | *6 | 0.5 | Comp. |
| 52 | UVI-6992 | 5.0 | *6 | 0.5 | Comp. |
| 53 | UVI-6992 | 5.0 | *6 | 0.5 | Comp. |
| 54 | UVI-6992 | 5.0 | *6 | 0.5 | Comp. |
| 55 | UVI-6992 | 5.0 | *6 | 0.5 | Comp. |
| 56 | UVI-6992 | 5.0 | *6 | 0.5 | Comp. |
| 57 | UVI-6992 | 5.0 | *6 | 0.5 | Comp. |

*6: Basin compound A,
*7: Basic compound B
Inv.: this invention,
Comp.: comparative example
Basic compound A: N-ethyldiethanol amine
Basic compound B: triisoproponol amine

### Curing of Active Ray Curable Composition

A film was formed and cured via the following method.

An obtained active ray curable composition was coated onto a PET film to bring a thickness of 6 μm, after which it was irradiated via ultraviolet rays of 800 m/cm² via a metal halide lamp within one second, to obtain a cured sample.

### Evaluation Method of Cured Sample

Physical properties of the obtained cured samples were evaluated via the following tests.

1) Each cured sample was measured for its hardness based on Scratch hardness (Pencil method) (JIS K 5400).

Ranking of hardness was in the order of (soft to hard: 6B -9H, and 6B was the softest and 9H was the hardest one.

2) Adhesion test via a cross-cut test of remaining ratio of tape peeling off (A cross-cut test of JIS K 5400): Onto the obtained cured sample with cross-cuts on the surface, adhesive tape (Scotch #250, produced by 3M Co.) was pressed to adhere via one pass with a 2 kg roller, after which the tape was peeled off at once, and any remaining cross-cuts (squares) were recorded.
3) Evaluation of Flexibility: Flex resistance of the obtained cured films were evaluated in accordance with “Bend test” of JIS K 5600.

### Evaluation of Storage Stability of Cured Film

The obtained cured composition was stored under a fluorescent light in a room at room temperature for one year, after which the Pencil Scratch Test was conducted as described above.

A: No major change in test results was noted.

B: A slight change was noted.

C: A major change was noted.

In the results, an “A” ranking was viable for practical applications.

### Evaluation of Viscosity

A: Viscosity of the cured composition was at most 50 mPa·s at 25° C.

B: Viscosity was 50-500 mPa·s.

C: Viscosity was at least 500 mPa·s.

The results are shown in Table 4.

TABLE 4

| | Pencil | | | | | |
|---|---|---|---|---|---|---|
| | scratch | | | Storage | | |
| Sam- | value | Adhesion | Flex | stability | | |
| ple | (Pencil | remaining | resistance | of cured | Viscos- | |
| No. | hardness) | ratio (%) | (φ: mm) | film | ity | Remarks |
| 21 | 3H | 75 | 3 mmφ | B | A | Inv. |
| 22 | 3H | 75 | *1 | B | A | Inv. |
| 23 | 3H | 75 | 2 mmφ | B | A | Inv. |
| 24 | 2H | 80 | 3 mmφ | B | A | Inv. |
| 25 | 2H | 75 | 2 mmφ | B | A | Inv. |
| 26 | 3H | 75 | 3 mmφ | B | A | Inv. |
| 27 | 2H | 80 | 2 mmφ | B | A | Inv. |
| 28 | 2H | 85 | *1 | B | A | Inv. |
| 29 | 2H | 80 | *1 | B | A | Inv. |
| 30 | 2H | 75 | *1 | B | A | Inv. |
| 31 | 2H | 80 | 2 mmφ | B | A | Inv. |
| 32 | 2H | 80 | *1 | B | A | Inv. |
| 33 | 3H | 80 | *1 | B | A | Inv. |
| 34 | 2H | 75 | *1 | B | A | Inv. |
| 35 | 2H | 80 | 2 mmφ | B | A | Inv. |
| 36 | 3H | 80 | *1 | B | A | Inv. |
| 37 | 3H | 80 | *1 | B | A | Inv. |
| 38 | 3H | 80 | *1 | B | A | Inv. |
| 39 | 2H | 80 | *1 | B | A | Inv. |
| 40 | 2H | 80 | *1 | B | A | Inv. |
| 41 | 3H | 80 | *1 | B | A | Inv. |
| 42 | 3H | 85 | *1 | B | A | Inv. |
| 43 | 3H | 80 | *1 | B | A | Inv. |
| 44 | 3H | 80 | *1 | B | A | Inv. |
| 45 | 3H | 80 | *1 | B | A | Inv. |
| 46 | 3H | 80 | *1 | B | A | Inv. |
| 47 | 3H | 80 | *1 | B | A | Inv. |
| 48 | 3H | 80 | *1 | B | A | Inv. |
| 49 | 3H | 80 | *1 | B | A | Inv. |
| 50 | 3H | 80 | *1 | B | A | Inv. |
| 51 | 2H | 65 | *2 | D | A | Comp. |
| 52 | H | 80 | 5 mmφ | D | A | Comp. |
| 53 | HB | 55 | 5 mmφ | C | A | Comp. |
| 54 | B | 40 | 3 mmφ | C | A | Comp. |
| 55 | 2H | 60 | *2 | D | A | Comp. |
| 56 | H | 75 | 7 mmφ | D | A | Comp. |
| 57 | H | 70 | 5 mmφ | D | D | Comp. |

*1: No crack was noted even at 1 mm φ
*2: Cracks were noted at 10 mm φ
Inv.: this invention,
Comp.: comparative example

From Table 4, it was proven that the samples of this invention exhibited sufficient hardness, and also adhesiveness was enhanced, and further, physical properties of the cured film were superior after long-term storage.

### Example 3

### Ink Components

Into a stainless steel beaker, poured were 5 weight parts of a dispersing agent (being PB 822, produced by Ajinomoto Fine-Techno Co., Inc.) polymerizable compounds described in Tables 5 and 6, after which the mixture was dissolved while heated and stirred on a 65° C. hot plate over one hour. Subsequently, 3 weight parts of various pigments were added to this solution, after which it was poured and stoppered tightly into a polyethylene (PE) bottle together with 200 g of 1 mm diameter of zirconia beads, and then dispersion treatment was conducted via a paint shaker for 2 hours. Subsequently, the zircinia beads were removed, and a combination of a photo polymerization initiator, basic compounds, and various additives such as a surface active agent were added as described in Tables 5 and 6. This solution was filtered using a 0.8 μm membrane filter to prevent printer head clogging, to prepare ink component sets.

TABLE 5

| | | | | | Another | |
|---|---|---|---|---|---|---|
| | Compound of | | Oxetane | | polymerizable | |
| | Formula (X) | | compound | | compound | |
| | | Added | | Added | | Added |
| Sam- | | amount | | amount | | amount |
| ple | | (weight | | (weight | | (weight |
| No. | Kind | part) | Kind | part) | Kind | part) |
| 101 | X-1 | 35 | OXT-212 | 65 | | |
| 102 | X-3 | 35 | OXT-212 | 65 | | |
| 103 | X-4 | 35 | OXT-212 | 65 | | |
| 104 | X-5 | 35 | OXT-212 | 65 | | |
| 105 | X-27 | 35 | OXT-212 | 65 | | |
| 106 | X-37 | 35 | OXT-212 | 65 | | |
| 107 | X-68 | 35 | OXT-212 | 65 | | |
| 108 | X-69 | 35 | OXT-212 | 65 | | |
| 109 | X-71 | 35 | OXT-212 | 65 | | |
| 110 | X-96 | 35 | OXT-212 | 65 | | |
| 111 | X-1 | 30 | OXT-221 | 50 | Celloxide 2021P | 20 |
| 112 | X-3 | 50 | OXT-221 | 30 | OXT-212 | 20 |
| 113 | X-4 | 40 | OXT-221 | 50 | OXT-212 | 10 |
| 114 | X-5 | 25 | OXT-221 | 65 | Celloxide 2021P | 10 |
| 115 | X-27 | 30 | OXT-221 | 60 | Celloxide 2021P | 10 |
| 116 | X-37 | 50 | OXT-221 | 40 | SEP-56 | 10 |
| 117 | X-68 | 50 | OXT-221 | 40 | SEP-72 | 10 |
| 118 | X-69 | 50 | OXT-221 | 40 | SEP-98 | 10 |
| 119 | X-71 | 50 | OXT-221 | 40 | SEP-102 | 10 |
| 120 | X-96 | 50 | OXT-221 | 40 | SEP-150 | 10 |
| 121 | X-3 | 55 | OXT-212 | 30 | EP-89 | 15 |
| 122 | X-3 | 40 | OXT-221 | 30 | SEP-56 | 30 |
| 123 | X-3 | 40 | OXT-221 | 30 | SEP-72 | 30 |
| 124 | X-69 | 40 | OXT-221 | 30 | SEP-98 | 30 |
| 125 | X-69 | 40 | OXT-221 | 30 | SEP-102 | 30 |
| 126 | X-69 | 40 | OXT-221 | 30 | SEP-150 | 30 |
| 127 | X-71 | 40 | OXT-221 | 30 | SEP-56 | 30 |
| 128 | X-71 | 40 | OXT-221 | 30 | SEP-150 | 30 |
| 129 | X-96 | 40 | OXT-221 | 30 | SEP-56 | 30 |
| 130 | X-96 | 60 | OXT-221 | 30 | EP-89 | 10 |
| 131 | | | OXT-221 | 65 | Celloxide 2021P | 35 |
| 132 | | | OXT-221 | 75 | Celloxide 2021P | 25 |
| 133 | | | OXT-223 | 65 | Vf7010 | 35 |
| 134 | | | OXT-221 | 75 | Vf7010 | 25 |
| 135 | | | OXT-221 | 65 | Celloxide 3000 | 35 |
| 136 | | | OXT-221 | 75 | Celloxide 3000 | 25 |
| 137 | | | OXT-221 | 45 | Epolead GT301 | 55 |

TABLE 6

| | Photo-acid generator | | | | | |
|---|---|---|---|---|---|---|
| Sample | | Added amount | *3 | *4 | *5 | |
| No. | Kind | (weight part) | Kind | Kind | Kind | Remarks |
| 101 | UVI-6992 | 5.0 | *9 | F1405 | 145P | Inv. |
| 102 | PI-1 | 5.0 | *9 | F1405 | 145P | Inv. |
| 103 | PI-1 | 5.0 | *9 | F1405 | 145P | Inv. |
| 104 | PI-1 | 5.0 | *9 | F1405 | 145P | Inv. |
| 105 | PI-1 | 5.0 | *9 | F1405 | 145P | Inv. |
| 106 | PI-1 | 5.0 | *9 | F1405 | 145P | Inv. |
| 107 | PI-2 | 5.0 | *10 | F1405 | 145P | Inv. |
| 108 | SP-152 | 5.0 | *10 | F1405 | 145P | Inv. |
| 109 | PI-1 | 5.0 | *9 | F1405 | 145P | Inv. |
| 110 | PI-1 | 5.0 | *9 | F1405 | 145P | Inv. |
| 111 | PI-1 | 5.0 | *9 | F1405 | 145P | Inv. |
| 112 | PI-1 | 5.0 | *9 | F1405 | 145P | Inv. |
| 113 | PI-2 | 5.0 | *9 | F178k | R100 | Inv. |
| 114 | UVI-6992 | 5.0 | *9 | F178k | R100 | Inv. |
| 115 | PI-1 | 5.0 | *10 | F178k | R100 | Inv. |
| 116 | PI-1 | 5.0 | *10 | F178k | R100 | Inv. |
| 117 | PI-1 | 5.0 | *10 | F178k | R100 | Inv. |
| 118 | PI-1 | 5.0 | *10 | F178k | R100 | Inv. |
| 119 | PI-1 | 5.0 | *10 | F178k | R100 | Inv. |
| 120 | PI-1 | 5.0 | *10 | F178k | R100 | Inv. |
| 121 | PI-1 | 5.0 | *9 | F1405 | 145P | Inv. |
| 122 | PI-1 | 5.0 | *9 | F1405 | 145P | Inv. |
| 123 | PI-1 | 5.0 | *9 | F1405 | 145P | Inv. |
| 124 | PI-1 | 5.0 | *10 | F1405 | 145P | Inv. |
| 125 | PI-1 | 5.0 | *10 | F1405 | 145P | Inv. |
| 126 | UVI-6992 | 5.0 | *9 | F178k | R100 | Inv. |
| 127 | SP-152 | 5.0 | *9 | F178k | R100 | Inv. |
| 128 | PI-1 | 5.0 | *10 | F178k | R100 | Inv. |
| 129 | PI-1 | 5.0 | *10 | F178k | R100 | Inv. |
| 130 | SP-152 | 5.0 | *10 | F178k | R100 | Inv. |
| 131 | UVI-6992 | 5.0 | *9 | F178k | R100 | Comp. |
| 132 | UVI-6992 | 5.0 | *9 | F178k | R100 | Comp. |
| 133 | UVI-6992 | 5.0 | *9 | F178k | R100 | Comp. |
| 134 | UVI-6992 | 5.0 | *9 | F178k | R100 | Comp. |
| 135 | UVI-6992 | 5.0 | *9 | F178k | R100 | Comp. |
| 136 | UVI-6992 | 5.0 | *9 | F178k | R100 | Comp. |
| 137 | UVI-6992 | 5.0 | *9 | F178k | R100 | Comp. |

*3: Basic compound (Added amount of 0.5 weight parts)
*4: Surface active agent (Added amount of 0.5 weight parts)
*5: Compatibilizer (Added amount of 0.5 weight parts)
*9: Basic compound A,
*10: Basic compound
Inv.: this invention,
Comp.: comparative example

The utilized pigments are listed below.

K: CI Pigment Black 7

C: CI Pigment Blue 15:3

M: CI Pigment Red 57:1

Y: CI Pigment Yellow 13

W: Titanium oxide (being an anatase type, at a diameter of 0.2 μm)

Lk: CI Pigment Black 7

Lc: CI Pigment Blue 15:3

Lm: CI Pigment Red 57:1

Ly: CI Pigment Yellow 13

The utilized compounds are listed below.

<Alicyclic Epoxy Compound>
Celloxide 2021P: produced by Daicel Chemical Industries Ltd.
Celloxide 3000: produced by Daicel Chemical Industries Ltd.
Epolead GT301: produced by Daicel Chemical Industries Ltd.

<Epoxidated Soybean Oil>
Vf70710: Vikoflex 7010 (produced by Atofina S. A.)

### <Oxetane Compound>

OXT-101: produced by Toagosei Co., Ltd.
OXT-221: produced by Toagosei Co., Ltd.
OXT-212: produced by Toagosei Co., Ltd.

[Photo-Acid Generator]
SP-152: Adekaoptomer SP-152, produced by Asahi Denka Co., Ltd.
UV16992: produced by The Dow Chemicals Co., being a 50% propione carbonate solution

[Surface Active Agent]
F178k: Megafax F178k, acryl oligomer containing a perfluoroalkyl group (produced by Dainippon Ink & Chemicals, Inc.)
F1405: Megafax 1405, ethyleneoxide adduct containing a perfluoroalkyl group (produced by Dainippon Ink & Chemicals, Inc.)

[Compatibilizer]
R100: Haritax R100 (Rosin modified maleic acid resin, produced by Harima Chemical Co., Ltd.)
145P: Haritax 145 (Rosin modified maleic acid resin, produced by Harima Chemical Co., Ltd.)

[Dispersing Agent]
PB 822: produced by Ajinomoto Fine-Techno Co., Inc.

[Basic Compound]

N-ethyldiethanol amine (Basic compound A)

Triisopropanol amine (Basic compound B)

### <<Ink-jet Image Forming Method>>

In an ink-jet recording apparatus featuring the constitution described in FIG. 1, which is equipped with piezo type ink-jet nozzles, each active ray curable composition ink prepared above was employed and the following image recording was continuously performed on 20 m of a long roll of recording medium having a width of 600 mm. The ink supply system was constituted of an ink tank, supply tubing, a pre-chamber ink tank immediately before the head, piping with a filter and a piezo head, of which a portion from the pre-chamber tank to the head was heat-insulated and heated to 50° C. Herein, the head portion was heated corresponding to the viscosity of each active ray curable composition ink, and driven so as to eject multi-sized dots of a liquid drop volume of 2-15 μl at a resolution of 720×720 dpi (dpi is a dot number per inch, that is, per 2.54 cm), whereby the above-described active ray curable composition ink was continuously ejected. Further, a recording medium was heated to 40° C. via a plane heater. Ink-jet ink, after having been deposited, was cured almost instantaneously (less than 0.5 second after deposition) via irradiation light source A, being a high pressure mercury lamp VZero 085 (manufactured by Integration Technology Corp.) or via irradiation light source B, being a metal halide lamp, MAL400NL (manufactured by Nippon Denchi Co., Ltd., at an electrical consumption of 3 kW-hr, 120 W/cm) on both sides of the carriage. The total ink layer thickness after image formation was determined to be in the range of 2.3-13 μm. Herein, ink-jet image formation was performed according to the above-described method under environments of 30° C. and 75% RH, and 25° C. and 25% RH.

Herein, illuminance of each irradiation light source was measured via a UVPF-Al manufactured by Iwasaki Electric Co., Ltd. at an accumulated illuminance of 254 nm.

Further, the details of the abbreviation of each recording medium described above follow.
Synthetic Paper: Synthetic Paper UPO FCS, produced by Upo Corp.
PVC: polyvinyl chloride

### Evaluation of Viscosity

Ink having a viscosity at 25° C. in the range of 7-40 mPa·s was ranked as “A”, and ink having a viscosity beyond this range was ranked as “C”.

### Evaluation of Ejection Behavior

After ink had been continuously ejected for 30 minutes, non-ejection of ink was visually evaluated.
A: No missed ejection was noted, and it was ranked at a level of “good”.
B: Occasional missed ejection was noted, but was within acceptable levels.
C: More missed ejection was noted, but was within a minimally acceptable level of image quality.
D: Numerous missed ejection arrears were noted, and was at an unviable commercial level.

“A” and “B” are considered to be in the acceptable range of practical usage.

### Evaluation of Ink-Jet Recorded Images

With respect to each image recorded via the above-described image forming method, the following evaluations were performed. MS-Ming style 6 point text characters were printed at a specific density, after which edge roughness of the printed characters was evaluated via magnification with a loupe, whereby text character quality was evaluated based on the following criteria.
A: No character-edge roughness was noted.
B: Slight character-edge roughness was noted.
C: More edge roughness was noted than in “B”, however, printed text characters could be easily read, and were rated at a minimally usable level.
D: Pronounced edge roughness was noted, and text characters appeared of low quality, and were rated at an unviable level.

“A” and “B” evaluations were in the acceptable range of practical usage.

### Color Mixing (Bleeding and Wrinkling)

Further, printing was performed so that individual dots of colors Y, M, C and K were adjacent to each other, and each color dot was observed through a loupe to visually evaluate any state of bleeding and wrinkling, whereby color mixing (namely bleeding) was evaluated based on the following criteria.
A: The shape of adjacent dots showed a true circle with no bleeding.
B: The shape of adjacent dots exhibited an almost true circle, showing minimal bleeding.
C: Adjacent dots showed slight bleeding, and the circular shape of adjacent dots was slightly deformed, and was rated at minimally viable level.
D: Adjacent dots showed obvious bleeding and were blended into each other, and wrinkles were generated at the superposed portions, and was rated at a commercially unviable level.

Ratings of “A” and “B” were in the acceptable level for practical usage.

### Evaluations of Hardness and Bending Resistance of Cured Film

Physical properties of cured sheets which were produced by printing via the above ink-jet image forming method, using cyan ink in which cyan pigment had been dispersed, were evaluated according to the following test methods.

1) Pencil scratch test: Hardness of each cured sheet sample was measured based on JIS K 5400. The ranks of hardness were in the order of soft to hard of 6B-9H, where 6B was the softest and 9H was the hardest one.

2) Evaluation of Flexibility: After the curable composition was ejected to coat onto sheets of synthetic paper (being Synthetic Paper Upo FGS, produced by Upo Corp.) to bring a layer thickness of 5 μm, the coated film was irradiated with ultraviolet rays of 800 mJ/cm² via a metal halide lamp within 1 second to prepare a cured image. Flex resistance of the obtained cured image was evaluated in accordance with “Bend test” of JIS K 5600.

### Evaluation of Ink Storage Stability

After prepared ink was tightly sealed in a vessel to be stored in a dark environment at room temperature for 1 month, image formation and tests similar to the above methods were performed to evaluate ink storage stability.

Samples with no significant variation of before and after test results, even after 1 month of aging, were ranked as A; samples showing significant variation in any one of the tests were ranked as B; and samples in which image formation was not possible due to viscosity increase were ranked as C. Ink rated at “A” as a level of storage stability, exhibiting no problems for practical use. The results are shown in following Table 7.

### Evaluation of Storage Stability of Cured Film

The obtained cured composition was stored under a fluorescent light in a room at room temperature for one year, after which Pencil Scratch Test was conducted as described above.

A: No major change in the test results was noted.

B: A slight change was noted.

C: A major change was noted.

In the results, an “A” ranking was viable for practical usage. The results are shown in following Table 7.

TABLE 7

| | 30° C. · 75% RH | | | | 25° C. · 25% RH | | | |
|---|---|---|---|---|---|---|---|---|
| Sample | PVC | | UPO-FGS | | PVC | | UPO-FGS | |
| No. | Character | Bleeding | Character | Bleeding | Character | Bleeding | Character | Bleeding |
| 101 | A | B | A | B | A | B | A | B |
| 102 | A | B | A | B | A | A | A | B |
| 103 | A | B | B | B | A | B | B | B |
| 104 | B | B | B | B | A | B | B | B |
| 105 | A | B | B | B | A | B | A | B |
| 106 | B | B | B | B | B | B | B | B |
| 107 | A | B | A | B | A | B | A | B |
| 108 | A | B | A | B | A | B | A | B |
| 109 | A | B | A | B | A | B | A | B |
| 110 | A | B | A | B | A | B | A | B |
| 111 | A | B | B | B | A | B | A | B |
| 112 | A | A | A | B | A | A | A | B |
| 113 | A | B | B | B | A | B | A | B |
| 114 | A | B | A | B | A | B | A | B |
| 115 | A | B | B | B | A | B | A | B |
| 116 | A | B | B | B | A | A | A | B |
| 117 | A | B | A | B | A | B | A | B |
| 118 | A | B | A | B | A | B | A | B |
| 119 | A | B | A | B | A | B | A | B |
| 120 | A | B | A | B | A | B | A | B |
| 121 | A | A | A | A | A | A | A | A |
| 122 | A | B | A | B | A | B | A | A |
| 123 | A | B | A | B | A | B | A | A |
| 124 | A | B | A | B | A | B | A | B |
| 125 | A | A | A | B | A | A | A | B |
| 126 | A | A | A | B | A | A | A | A |
| 127 | A | A | A | B | A | A | A | A |
| 128 | A | A | A | B | A | A | A | A |
| 129 | A | A | A | B | A | A | A | A |
| 130 | A | A | A | A | A | A | A | A |
| 131 | C | B | B | B | B | B | B | B |
| 132 | C | B | B | B | B | B | B | B |
| 133 | D | D | D | D | C | C | C | C |
| 134 | D | D | D | D | C | C | C | C |
| 135 | C | B | B | B | B | B | B | B |
| 136 | C | B | B | B | B | B | B | B |
| 137 | C | C | C | C | B | C | B | C |

| | Hardness | | Ink | Storage | | | |
|---|---|---|---|---|---|---|---|
| Sample | of cured | Flex resistance | ejection | stability | | Viscosity | |
| No. | film | φ: mm | behavior | of ink | *1 | of ink | Remarks |
| 101 | 3H | 3 mmφ | A | B | B | A | Inv. |
| 102 | 3H | No crack was noted even at 1 mm φ | A | B | B | A | Inv. |
| 103 | 3H | 2 mmφ | A | B | B | A | Inv. |
| 104 | 2H | 3 mmφ | A | B | B | A | Inv. |
| 105 | 2H | 2 mmφ | B | B | B | A | Inv. |
| 106 | 3H | 3 mmφ | A | B | B | A | Inv. |
| 107 | 2H | 2 mmφ | A | B | B | A | Inv. |
| 108 | 2H | No crack was noted even at 1 mm φ | A | B | B | A | Inv. |
| 109 | 2H | No crack was noted even at 1 mm φ | A | B | B | A | Inv. |
| 110 | 2H | No crack was noted even at 1 mm φ | A | B | B | A | Inv. |
| 111 | 2H | 2 mmφ | A | B | B | A | Inv. |
| 112 | 2H | No crack was noted even at 1 mm φ | A | B | B | A | Inv. |
| 113 | 3H | No crack was noted even at 1 mm φ | B | B | B | A | Inv. |
| 114 | 2H | No crack was noted even at 1 mm φ | B | B | B | A | Inv. |
| 115 | 2H | 2 mmφ | B | B | B | A | Inv. |
| 116 | 3H | No crack was noted even at 1 mm φ | B | B | B | A | Inv. |
| 117 | 3H | No crack was noted even at 1 mm φ | A | B | B | A | Inv. |
| 118 | 3H | No crack was noted even at 1 mm φ | A | B | B | A | Inv. |
| 119 | 2H | No crack was noted even at 1 mm φ | A | B | B | A | Inv. |
| 120 | 2H | No crack was noted even at 1 mm φ | A | B | B | A | Inv. |
| 121 | 3H | No crack was noted even at 1 mm φ | A | B | B | A | Inv. |
| 122 | 3H | No crack was noted even at 1 mm φ | A | B | B | A | Inv. |
| 123 | 3H | No crack was noted even at 1 mm φ | A | B | B | A | Inv. |
| 124 | 3H | No crack was noted even at 1 mm φ | A | B | B | A | Inv. |
| 125 | 3H | No crack was noted even at 1 mm φ | A | B | B | A | Inv. |
| 126 | 3H | No crack was noted even at 1 mm φ | A | B | B | A | Inv. |
| 127 | 3H | No crack was noted even at 1 mm φ | B | B | B | A | Inv. |
| 128 | 3H | No crack was noted even at 1 mm φ | B | B | B | A | Inv. |
| 129 | 3H | No crack was noted even at 1 mm φ | B | B | B | A | Inv. |
| 130 | 3H | No crack was noted even at 1 mm φ | B | B | B | A | Inv. |
| 131 | 2H | *2 | B | C | D | A | Comp. |
| 132 | H | 5 mmφ | B | C | D | A | Comp. |
| 133 | HB | 5 mmφ | B | C | C | A | Comp. |
| 134 | B | 3 mmφ | B | C | C | A | Comp. |
| 135 | 2H | *2 | A | C | D | A | Comp. |
| 136 | H | 7 mmφ | A | C | D | A | Comp. |
| 137 | H | 5 mmφ | D | C | D | D | Comp. |

*1: Storage stability of cured film
*2: Cracks were noted at 10 mm φ
Inv.: this invention,
Comp.: comparative example

From the results in Table 7, it was proven that the samples of this invention exhibited superior ink ejection behavior, as well as enhanced image quality, and further, excellent ink storage stability. It was also proven that the samples of this invention showed excellent curing capability regardless of environmental conditions, and exhibited superiority in storage stability of formed cured film.

1. An active ray curable composition comprising a compound represented by Formula (X): 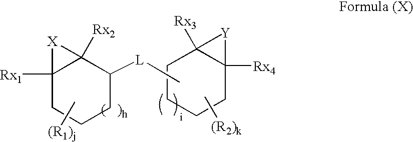
wherein X and Y are each independently an oxygen atom or a sulfur atom, L is a single bond or a divalent connecting group, R_{X1}, R_{X2}, R_{X3} and R_{X4} are each independently a hydrogen atom or an alkyl group, R₁ and R₂ are each independently a substituent group, h and i are each independently an integer of 0-2, j and k are each independently an integer of 0-9, a is 0 or 1, and when it is 0, L is combined to a carbon atom substituted with R_{X3}.

2. The active ray curable composition described in claim 1, wherein in the compound represented by Formula (X), either R_{X1} or R_{X2} is an alkyl group and the other one being a hydrogen atom, and further, either R_{X3} or R_{X4} is an alkyl group and the other one is a hydrogen atom.

3. The active ray curable composition described in claim 2, wherein in the compound represented by Formula (X), either R_{X1} or R_{X2} is an alkyl group exhibiting 1-3 carbon atoms and the other one is a hydrogen atom, and further, either R_{X3} or R_{X4} is an alkyl group exhibiting 1-3 carbon atoms and the other one is a hydrogen atom.

4. The active ray curable composition described in claim 1, further comprises an oxetane compound.

5. The active ray curable composition described in claim 4, wherein the oxetane compound does not have a substituent group in a 2-position of an oxetane ring.

6. The active ray curable composition described in claim 4, wherein the oxetane compound having no substituent group in the 2-position of the oxetane ring is a multifunctional oxetane compound having two or more oxetane rings.

7. The active ray curable composition described in claim 1, further comprising a compound represented by following Formula (A): 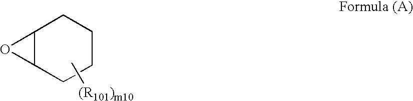
wherein R101 is a substituent group not having a reactive functional group of being cationic polymerizable or radical polymerizable, and m10 is 1, 2, 3, or 4.

8. The active ray curable composition described in claim 1, further comprises a polymerization initiator.

9. The active ray curable composition described in claim 8, wherein the polymerization initiator is a compound which generates an acid when irradiated with active rays.

10. The active ray curable composition described in claim 9, wherein the compound which generates an acid when irradiated with active rays is an onium salt compound.

11. The active ray curable composition described in claim 10, wherein the compound which generates an acid when irradiated with active rays is a sulfonium salt compound.

12. The active ray curable composition described in claim 11, wherein the sulfonium compound is one represented by Formulas (I-1), (I-2) or (I-3): 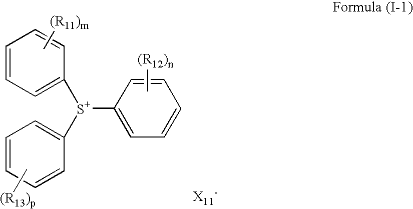
wherein R₁₁, R₁₂, and R₁₃ are each a substituent group, and m, n, and p are each an integer of 0-2, and X₁₁⁻ is a counter ion, 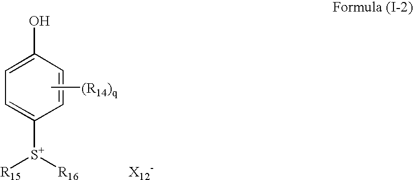
wherein R₁₄ is a substituent group, and q is an integer of 0-2, R₁₅ and R₁₆ are each a substituted or unsubstituted alkyl group, a substituted or unsubstituted alkenyl group, a substituted or unsubstituted alkynyl group, or a substituted or unsubstituted aryl group, and X₁₂⁻ is a counter ion, 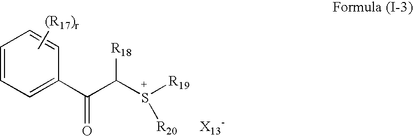
wherein R₁₇ is a substituent group, and r is an integer of 0-2, R₁₈ is a hydrogen atom or a substituted or unsubstituted alkyl group, and R₁₉ and R₂₀ are each a substituted or unsubstituted alkyl group, a substituted or unsubstituted alkenyl group, a substituted or unsubstituted alkynyl group, or a substituted or unsubstituted aryl group, and X₁₃⁻ is a counter ion.

13. The active ray curable composition described in claim 1, exhibits a viscosity of 1-500 mPa·s at 25° C.

14. A polymerizing method comprising the steps of:
(a) ejecting an active ray curable composition described in claim 1 on a recording medium, and
(b) polymerizing the ejected active ray curable composition with active rays.

15. An active ray curable ink comprises an active ray curable composition described in claim 1.

16. The active ray curable ink described in claim 15, exhibits a viscosity of 7-40 mPa·s at 25° C.

17. The active ray curable ink described in claim 15, contains a pigment.

18. An image forming method comprising the steps of:
(a) ejecting imagewise an active ray curable ink described in claim 15 onto a recording medium from an ink-jet recording head, and
(b) forming an image on the recording medium by curing the active ray curable ink,
wherein the active ray curable ink is cured by radiation of active rays during 0.001-1.0 second after deposition of the active ray curable ink onto the recording medium.

19. The image forming method described in claim 18, wherein a minimum ink droplet volume ejected from each nozzle of the ink-jet recording head is 2-15 pl.

20. The image forming method described in claim 18, wherein the active ray curable ink is ejected after the active ray curable ink and the ink-jet recording head are heated to 35-100° C.

21. The image forming method described in claim 18, wherein the active ray curable ink is ejected onto the recording medium heated to 35-60° C.

22. An ink-jet recording apparatus being utilized in an image forming method described in claim 18.
